(19) United States
(12) Patent Application Publication
(10) Pub. No.: US 2022/0324891 A1
(43) Pub. Date: Oct. 13, 2022

(54) COMPOSITIONS AND METHODS FOR PREPARING REGIO- AND STEREOSELECTIVE ALICYCLIC ALKENE ISOTOPOLOGUES AND STEREOISOTOPOMERS

(71) Applicant: University of Virginia Patent Foundation, Charlottesville, VA (US)

(72) Inventors: Walter Dean Harman, Earlysville, VA (US); Jacob Smith, Burke, VA (US); Katy Wilson, Charlottesville, VA (US); Kevin David Welch, Charlottesville, VA (US); Justin H. Wilde, Bristol, CT (US)

(21) Appl. No.: 17/619,827

(22) PCT Filed: Jun. 17, 2020
(86) PCT No.: PCT/US2020/038163

§ 371 Date: Dec. 16, 2021

Related U.S. Application Data
(60) Provisional application No. 62/862,452, filed on Jun. 17, 2019.

(51) Int. Cl. C07F 11/00

(52) U.S. Cl. CPC C07F 11/00; C07B 2200/05; C07B 2200/07

(57) ABSTRACT

A method for preparing isotopologues and/or stereoisotopomers of cyclic and heterocyclic alkenes and dienes is described. The method provides regio- and/or stereospecific addition of hydrogen, deuterium, tritium and a variety of other substituents to arenes, heteroarenes, and alicyclic compounds that have multiple carbon-carbon double bonds, thereby providing discrete isotopologues and stereoisotopomers of cyclic and heterocyclic alkenes and dienes with high isotopic purity and in high enantiomeric excess. Also described are isotopologues and stereoisotopomers of cyclic and heterocyclic alkenes and dienes, such as isotopologues and stereoisotopomers of cyclohexene and tetrahydropyridine, as well as products thereof, such as isotopologues and stereoisotopomers of piperidines and piperidine-containing compounds, such as methylphenidate. In addition, a method of determining the absolute configuration of stereoisotopomers of cyclohexenes is described.

### CROSS REFERENCE TO RELATED APPLICATION

The presently disclosed subject matter claims the benefit of U.S. Provisional Patent Application Ser. No. 62/862,452, filed Jun. 17, 2019; the disclosure of which is incorporated herein by reference in its entirety.

### GOVERNMENT INTEREST

This invention was made with government support under Grant No. GM132205 awarded by the National Institutes of Health. The government has certain rights in the invention.

### TECHNICAL FIELD

The presently disclosed subject matter provides a method for preparing isotopologues and stereoisotopomers of cyclic and heterocyclic alkenes and dienes in a regio- and stereoselective manner, as well as providing the isotopologues and stereoisotopomers themselves, including isotopologues and stereoisotopomers of cyclohexene and tetrahydropyridine, and their products, such as isotopologues and stereoisotopomers of methylphenidate. The presently disclosed subject matter further provides a method of determining the absolute configuration of stereoisotopomers of cyclohexenes.

### ABBREVIATIONS

° C.=degrees Celsius
%=percentage
η²=dihapto
Ac=acetyl
API=active pharmaceutical ingredient
D=deuterium
DCM=dichloromethane
DFT=discrete Fourier transform
DKIE=deuterium kinetic isotope effect
DMAP=4-(dimethylamino)pyridine
DMDO=dimethyldioxirane
DME=1,2-dimethoxyethane
DPhAT=diphenylammonium triflate
ee=enantiomeric excess
Et₂O=diethyl ether
g=gram
HOTf=trifluoromethanesulfonic acid
IP=isotopic purity
KIE=kinetic isotope effect
M=molar
MeCN=acetonitrile
Melm=N-methyl imidazole
MeOD=deuterated methanol
MeOH=methanol
mL=milliliter
mM=millimolar
mmol=millimole
Mo=molybdenum
NaBD₄=sodium borodeuteride
NaBH₄=sodium borohydride
NaCN=sodium cyanide
NHE=normal hydrogen electrode
NMR=nuclear magnetic resonance
NOE=nuclear Overhauser effect
NOESY=nuclear Overhauser effect spectroscopy
Os=osmium
Re=rhenium
T=tritium
THP=tetrahydropyridine
Ts=toluenesulfonyl (or tosyl)
Tp=trispyrazolylborate
V=volts
W=tungsten

### BACKGROUND

The hydrogen isotopes deuterium (D) and tritium (T) have become essential tools of chemistry, biology, and medicine.¹ Beyond their widespread use in spectroscopy, mass spectrometry, and mechanistic and pharmacokinetic studies, there has been considerable interest in incorporating deuterium into the active pharmaceutical ingredient (API) of drugs.¹ The deuterium kinetic isotope effect (DKIE), which compares the rate of a chemical reaction for a compound to its deuterated counterpart, can be dramatic.¹⁻³ Consequently, the strategic replacement of hydrogen with deuterium can affect both the rate of metabolism and distribution of metabolites for a compound,⁴ improving the efficacy and safety of the drug. Deutetrabenazine, a promising treatment for Huntington's disease,⁵ recently became the first deuterated drug to win FDA-approval.

Significantly, the pharmacokinetics of a deuterated compound depends on the location(s) where the deuterium/hydrogen replacement has occurred. While methods exist for deuterium incorporation at both early and late stages of a drug's synthesis,⁶⁻⁷ these processes are often unselective and the stereoisotopic purity can be difficult to measure.⁷⁻⁸ Accordingly, there is an ongoing need for systematic methods for the preparation of pharmacologically active compounds as discrete stereoisotopomers. Such methods could improve pharmacological and toxicological properties of drugs and provide new mechanistic information related to their distribution and metabolism in the body.

### SUMMARY

In some embodiments, the presently disclosed subject matter provides a method of preparing an isotopologue or a stereoisotopomer of a cyclic or heterocyclic alkene or diene, the method comprising: (a) providing a first metal complex, wherein said first metal complex comprises a transition metal selected from tungsten (W), rhenium (Re), osmium (Os), and molybdenum (Mo) and a dihapto-coordinated ligand, wherein said dihapto-coordinated ligand is selected from an arene, a heteroarene or a salt thereof, and an alicyclic compound comprising at least two carbon-carbon double bonds; (b) reducing the dihapto-coordinated ligand, optionally wherein said reducing comprises contacting said first metal complex sequentially with at least a first reagent and a second reagent, wherein said first reagent is a Bronsted acid or a deuterated or tritiated analogue thereof, and wherein the second reagent is a nucleophilic reagent, thereby forming a second metal complex comprising the transition metal and a dihapto-coordinated cyclic or heterocyclic alkene or diene; and (c) decomplexing the cyclic or heterocyclic alkene or diene from the second metal complex, wherein said decomplexing optionally comprises contacting the second metal complex with an oxidant, thereby providing the isotopologue or stereoisotopomer of a cyclic or heterocyclic alkene or diene, wherein said isotopologue or stereoisotopomer comprises at least one deuterium or tritium.

In some embodiments, the transition metal is W. In some embodiments, providing the first metal complex comprises one of: contacting tungsten trispyrazolylborate nitroso trimethylphosphine dihapto-coordinated benzene (WTp(NO)(PMe₃)(η²-benzene)) with an arene, an alicyclic diene or an alicyclic triene, thereby forming a WTp(NO)(PMe₃)(η²-arene), a WTP(NO)(PMe₃)(η²-diene) or a WTp(NO)(PMe₃)(η²-triene); contacting a tungsten trispyrazolylborate nitroso trialkylphosphine halide complex with an arene in the presence of an alkali metal, optionally sodium, thereby forming a WTp(NO)(PMe₃)(η²-arene) complex; and contacting WTp(NO)(PMe₃)(η²-benzene) with a pyridine borane and contacting the resulting complex with a Bronsted acid to form a WTp(NO)(PMe₃)(η₂-pyridinium) salt, optionally followed by contacting the WTp(NO)(PMe₃)(η₂-pyridinium) salt with an anhydride or acid chloride in the presence of a weak base.

In some embodiments, the dihapto-coordinated ligand of the first metal complex is selected from the group comprising benzene, naphthalene, anthracene, cyclopentadiene, cyclohexadiene, furan, pyrrole, pyridine, a pyridinium salt, thiophene, and deuterated, tritiated, and/or substituted analogues thereof; optionally wherein the arene is selected from the group comprising benzene, substituted benzene, naphthalene, substituted naphthalene, furan, a pyridinium salt, a substituted pyridinium salt and deuterated or tritiated analogues thereof.

In some embodiments, the first metal complex comprises a dihapto-coordinated arene or a dihapto-coordinated heteroarene or salt thereof, and wherein step (b) comprises: (b1) contacting the first metal complex sequentially with a first reagent and a second reagent, wherein the first reagent is a Bronsted acid or a deuterated or tritiated analogue thereof, and wherein the second reagent is a nucleophilic reagent, thereby forming an intermediate metal complex comprising a dihapto-coordinated cyclic or heterocyclic diene ligand; and (b2) contacting the intermediate metal complex comprising the dihapto-coordinated cyclic or heterocyclic diene ligand sequentially with a third reagent and a fourth reagent, wherein the third reagent is a Bronsted acid or a deuterated or tritiated analogue thereof, and wherein the fourth reagent is a nucleophilic reagent; thereby forming the second metal complex, wherein said second metal complex comprises a dihapto-coordinated cyclic or heterocyclic alkene ligand.

In some embodiments, the first reagent and the third reagent are each independently a strong acid or a deuterated or tritiated analogue thereof, wherein said strong acid is selected from the group comprising diphenylammonium triflate (DPhAT), trifluoromethanesulfonic acid (HOTf); sulfuric acid (H₂SO₄), hexafluorophosphoric acid (HPF₆), tetrafluoroboric acid (HBF₄), hydrochloric acid (HCl), and hydrobromic acid (HBr). In some embodiments, the contacting with the first reagent in step (b1) and the contacting with the third reagent in step (b2) is performed in an ether, nitrile, or ester solvent at a temperature between about −60° C. and about −20° C., optionally at about −30° C.

In some embodiments, at least one of the second reagent and the fourth reagent is a hydride or a deuteride reagent selected from sodium borohydride (NaBH₄) and sodium borodeuteride (NaBD₄); wherein when the at least one of the second reagent and the fourth reagent is NaBH₄, the contacting with the at least one of the second reagent and the fourth reagent is performed in methanol; and wherein when the at least one of the second reagent and the fourth reagent is NaBD₄, the contacting with the at least one of the second reagent and the fourth reagent is performed in deuterated methanol or a mixture of acetonitrile and 15-crown-5 ether.

In some embodiments, the contacting with the at least one of the second reagent and the fourth reagent is performed at a temperature between about −60° C. and about −20° C., optionally at about −60° C. In some embodiments, the second reagent and the fourth reagents are each independently selected from a hydride reagent and a deuteride reagent.

In some embodiments, at least one of the second reagent and the fourth reagent is selected from the group comprising a cyanide salt, an alkoxide salt, an alkynide salt, an alkyl or aryl magnesium halide, a dialkylzinc, an enolate, a phosphine, a primary amine, and a secondary amine. In some embodiments, at least one of steps (b1) and (b2) comprise a stereoselective addition of at least one of a proton, a deuteron, a triton, or a nucleophile, optionally the stereoselective addition of both a proton, deuteron or triton and a nucleophile, further optionally wherein said nucleophile is a hydride or a deuteride. In some embodiments, the method provides an isotopologue or a stereoisotopomer having at least about 75% isotopic purity, optionally at least about 90% isotopic purity.

In some embodiments, the dihapto-coordinated ligand of the first metal complex is an N-acylated pyridinium salt, a N-tosylated pyridinium salt, or an N-acylated or N-tosylated substituted pyridinium salt, and the method provides an isotopologue or a stereoisotopomer of a tetrahydropyridine (THP). In some embodiments, the method further comprises contacting the isotopologue or stereoisotopomer of the THP with a hydrogenation reagent, thereby providing an isotopologue or a stereoisotopologue of a piperidine, optionally wherein the piperidine is methylphenidate.

In some embodiments, the dihapto-coordinated ligand of the first metal complex is an arene selected from benzene, benzene-d₆, a substituted benzene, and an exhaustively deuterated, substituted benzene; and wherein step (b1) comprises: (b1-i) contacting the first metal complex with a Bronsted acid or a deuterated Bronsted acid, thereby forming a metal complex comprising a dihapto-coordinated benzenium ligand; and (b1-ii) contacting the metal complex comprising the dihapto-coordinated benzenium ligand with a nucleophilic reagent, thereby forming the intermediate metal complex, wherein said intermediate metal complex comprises a dihapto-coordinated cyclohexadiene ligand; wherein step (b2) comprises: (b2-i) contacting the intermediate metal complex with a Bronsted acid or a deuterated Bronsted acid, thereby forming a metal complex comprising a dihapto-coordinated allyl ligand; and (b2-ii) contacting the metal complex comprising the dihapto-coordinated allyl ligand with a nucleophilic reagent, thereby forming the second metal complex, wherein said second metal complex comprises the dihapto-coordinated cyclohexene ligand; and wherein step (c) comprises decomplexing the dihapto-coordinated cyclohexene ligand, optionally wherein the decomplexing comprises contacting the second metal complex with an oxidant, thereby providing the isotopologue or stereoisotopomer of a cyclohexene, wherein said isotopologue or stereoisotopomer comprises at least one deuterium.

In some embodiments, one of more of steps (b1-i), (b1-ii), (b2-i), and (b2-ii) are stereoselective. In some embodiments, the method provides an isotopologue or a stereoisotopomer of a cyclohexene having at least about 75% isotopic purity, optionally at least about 90% isotopic purity.

In some embodiments, the dihapto-coordinated ligand of the first metal complex is benzene or benzene-d₆ and the contacting of step (b1-i) comprises endo-selective protonation or deuteration of the benzene or benzene-d₆ ligand. In some embodiments, the nucleophilic reagent of step (b1-i) is a hydride or a deuteride reagent and the contacting of step (b1-i) comprises exo-selective addition of a hydride or deuteride to the benzenium ligand. In some embodiments, the contacting of step (b2-i) comprises exo-selective protonation or deuteration of the cyclohexadiene ligand. In some embodiments, the nucleophilic reagent of step (b2-ii) is a hydride or a deuteride reagent and the contacting of step (b2-ii) comprises selective addition of a hydride or deuteride to the allyl ligand anti to the metal of the metal complex comprising the dihapto-coordinated allyl ligand.

In some embodiments, the arene is benzene or a substituted benzene and the isotopologue or stereoisotopomer is a d₁-, d₂-, d₃-, or d₄-cyclohexene. In some embodiments, the arene is benzene-d₆ and the isotopologue or stereoisotopomer is a d₆-, d₇-, or d₈-cyclohexene. In some embodiments, the arene is a substituted benzene, optionally wherein the substituted benzene comprises a substituent selected from alkyl, perfluoroalkyl, cyano, a sulfone, and a sulfonamide. In some embodiments, the method further comprises contacting the isotopologue or stereoisotopomer of the cyclohexene with a dioxirane, optionally dimethyldioxirane (DMDO), thereby converting the isotopologue or stereoisoptopomer of the cyclohexene into an epoxide. In some embodiments, the method provides a stereoisotopomer of a cyclohexene with a stereoselectivity of 22:1 or more.

In some embodiments, the decomplexing comprises contacting the second metal complex with an oxidant, wherein said oxidant is a one electron oxidant, optionally wherein the oxidant is selected from the group comprising 2,3-dichloro-5,6-dicyano-1,4-benzoquinone (DDQ), an iron (Fe) (III) compound, nitrosonium hexafluorophosphate (NOPF₆), a copper (Cu) (II) salt, silver (Ag) (I) salt, or another oxidant with a potential greater than about 0.5 Volts (V) versus a normal hydrogen electrode (NHE).

In some embodiments, the isotopologue or stereoisotopomer of the cyclic or heterocyclic alkene or diene is a synthetic intermediate of a deuterated active pharmaceutical ingredient.

In some embodiments, the presently disclosed subject matter provides an isotopologue or stereoisotopomer prepared according to the presently disclosed method.

In some embodiments, the presently disclosed subject matter provides an isotopologue or stereoisotopomer of a cyclohexene or a substituted cyclohexene, wherein said isotopologue or stereoisotopomer comprises at least one cyclohexene ring carbon substituted by hydrogen and at least one cyclohexene ring carbon substituted by deuterium or tritium, subject to the proviso that said isotopologue or stereoisotopomer is not cyclohex-1-ene-1,2-d₂; cyclohex-1-ene-1-d; (R)-cyclohex-1-ene-3-d; or (3R,4R,5S,6S)-cyclohex-1-ene-3,4,5,6-d₄. In some embodiments, said isotopologue or stereoisotopomer has an isotopic purity of at least 75%. In some embodiments, said isotopologue or stereoisotopomer is a stereoisotopomer having an enantiomeric excess of about 80% or more.

In some embodiments, the isotopologue or stereoisotopomer has a structure of one of Formulas (Ia), (Ib), (IIa), (IIb), (IIIa), and (IIIb):

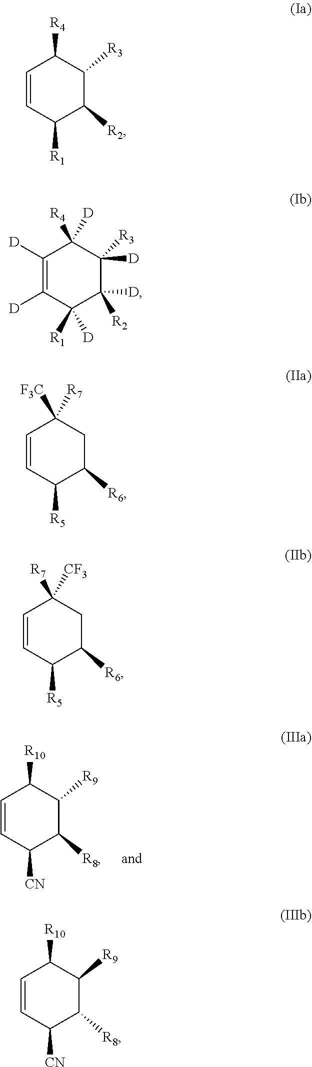

wherein: each of R₁, R₂, R₃, R₄, R₅, R₆, R₇, R₈, R₉, and R₁₀ is independently selected from H and D, subject to the proviso that for Formula (Ia), at least one of R₁-R₄ is D; that for Formula (Ib), at least one of R₁-R₄ is H; that for Formulas (IIa) and (IIb) at least one of R₅-R₇ is D; and that for Formula (IIIa) and (IIIb), at least one of R₈-R₁₀ is D.

In some embodiments, the isotopologue or stereoisotopomer has a structure of Formula (Ia), wherein one, two, three, or all four of R₁R₂, R₃, and R₄ is D. In some embodiments, the isotopologue or stereoisotopomer has a structure of Formula (Ib), wherein R₁ and R₂ are D and R₃ and R₄ are H; R₁ is D and R₂, R₃, and R₄ are each H; or R₁-R₄ are each H. In some embodiments, the isotopologue or stereoisotopomer has a structure of Formula (IIa), wherein one or both of R₅ and R₆ is D and R₇ is H. In some embodiments, the isotopologue or stereoisotopomer has a structure of Formula (IIb), wherein R₅ and R₆ are each D and R₇ is H or D. In some embodiments, the isotopologue or stereoisotopomer has a structure of Formula (IIIa), wherein one of R₈-R₁₀ is D and the other two of R₈-R₁₀ are each H; or wherein R₈ and R₉ are each D and R₁₀ is H. In some embodiments, the isotopologue or stereoisotopomer has a structure of Formula (IIIb), wherein R₁₀ is H; and one of R₈ and R₉ is D and one of R₈ and R₉ is H.

In some embodiments, the presently disclosed subject matter provides an isotopologue or stereoisotopomer of tetrahydropyridine or a substituted tetrahydropyridine, wherein the isotopologue or stereoisotopomer comprises one, two, three, four, five, six, or seven deuteriums attached to tetrahydropyridine ring carbon atoms. In some embodiments, the isotopologue or stereoisotopomer has a structure of one of Formulas (IVa) and (IVb):

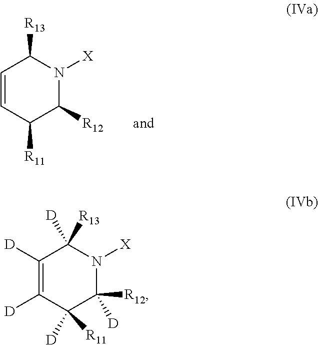

wherein: X is H, D, acyl, or tosyl; and each of R₁₁, R₁₂, and R₁₃ is independently selected from H and D, subject to the proviso that for Formula (IVa), at least one of R₁₁, R₁₂, and R₁₃ is D; and for Formula (IVb), at least one of R₁₁, R₁₂, and R₁₃ is H; or a salt thereof. In some embodiments, the isotopologue or stereoisotopomer has a structure of one of (Va) and (Vb):

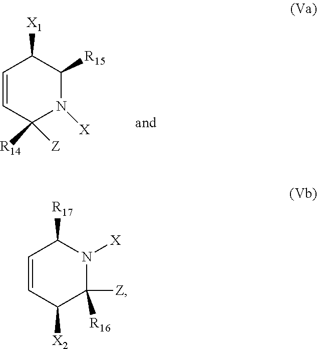

wherein: X is H, D, acyl, or tosyl; X₁ and X₂ are each selected from the group consisting of H, D, CN, alkyl, substituted alkyl, alkoxy, aryloxy, —NHR₂₄, —N(R₂₄)₂; and —P(R₂₄)₃; Z has a structure of the formula

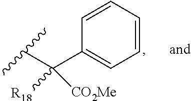

each of R₁₄, R₁₅, R₁₆, R₁₇, and R₁₈ is independently selected from H and D; and each R₂₄ is independently selected from alkyl, aralkyl, and aryl; subject to the proviso that for Formulas (Va) at least one of R₁₄, R₁₅, and X₁ is D; and that for Formula (Vb) at least one of R₁₆, R₁₇, and X₂ is D; or a salt thereof. In some embodiments, said isotopologue or stereoisotopomer an isotopic purity of at least 75%.

In some embodiments, the presently disclosed subject matter provides an isotopologue or a stereoisotopomer of methylphenidate or a 6-trifluoromethyl substituted derivative thereof, wherein the isotopologue or stereoisotopomer has a structure of Formula (VI):

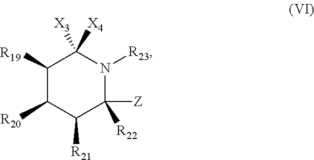

wherein X₃ and X₄ are each selected from H, D, and —CF₃; Z has a structure of the formula:

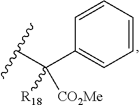

and
each of R₁₈, R₁₉, R₂₀, R₂₁, R₂₂ and R₂₃ is selected from H and D; or a salt thereof; and subject to the proviso that when one of X₃ and X₄ is —CF₃, the other of X₃ and X₄ is H or D; and that when neither of X₃ and X₄ is —CF₃, X₃ is H and X₄ is H or D; and that at least one of R₂₁, R₂₂, and X₄ is D. In some embodiments, said isotopologue or stereoisotopomer an isotopic purity of at least 75%.

In some embodiments, the presently disclosed subject matter provides a method determining an absolute configuration of a stereoisotopomer of a cyclohexene, wherein the method comprises: (a) contacting the stereoisotopomer of the cyclohexene with a tungsten metal complex, wherein said tungsten metal complex is a resolved form of WTp(NOMe)(PMe₃)(η²-benzene) and wherein the contacting results in ligand exchange between the benzene and the cyclohexene, thereby providing a tungsten metal complex wherein the stereoisotopomer of the cyclohexene is dihapto-coordinated to tungsten; (b) collecting a proton nuclear magnetic resonance (NMR) spectrum of the tungsten metal complex comprising the dihapto-coordinated stereoisotopomer of the cyclohexene; and (c) comparing the proton NMR spectrum collected in step (b) to a proton NMR spectrum of the corresponding tungsten metal complex wherein the dihapto-coordinated ligand is a non-isotopically enriched cyclohexene; thereby determining the absolute configuration of the stereoisotopomer.

Accordingly, it is an object of the presently disclosed subject matter to provide a method for preparing an isotopologue or a stereoisotopomer of a cyclic or heterocyclic alkene or diene; to provide isotopologues or stereoisotopomers of cyclic and heterocyclic alkenes and dienes, to provide isotopologues or stereoisotopomers of piperidines, such as methylphenidate, and to provide a method of determining the absolute configurations of stereoisotopomers of cyclohexenes.

An object of the presently disclosed subject matter having been stated hereinabove, and which is achieved in whole or in part by the presently disclosed subject matter, other objects will become evident as the description proceeds when taken in connection with the accompanying drawings and examples as best described hereinbelow.

### BRIEF DESCRIPTION OF THE DRAWINGS

FIG. 1A is a schematic drawing of an exemplary method for the selective deuteration of benzene via stepwise reduction involving the sequential addition of hydrogen or deuterium cations (H/D⁺) and hydrogen or deuterium anions (hydride or deuteride, H/D⁻) to a tungsten complex to provide select isotopologues of a complexed cyclohexene in a regio- and stereoselective manner.

FIG. 1B is a schematic drawing of a dihapto (η²)-coordinated benzene metal complex, i.e. tungsten trispyrazolylborate nitroso trimethylphosphine benzene (WTp(NO)(PMe₃)(η²-benzene) (1).

FIG. 2A is a schematic drawing showing the sequential reduction of benzene to cyclohexene starting from complex 1 shown in FIG. 1B.

FIG. 2B is a schematic drawing showing the solid-state molecular structure from a single-crystal X-ray diffraction study and the relevant nuclear Overhauser effect (NOE) interactions for methylated cyclohexene complex 9.

FIG. 3A is a schematic drawing showing the synthesis of isotopologues of cyclohexene comprising 2, 4, or 6 deuterium atoms (i.e., d₂, d₄, and d₆ isotopologues) from tungsten complexes comprising dihapto-complexed benzene or dihapto-complexed deuterated benzene (benzene-d₆).

FIG. 3B is a schematic drawing showing the synthesis of isotopologues of cyclohexene comprising 1 or 2 deuterium atoms (i.e., d₁ and d₂ isotopologues) from tungsten complexes comprising dihapto-complexed benzene, dihapto-complexed cyclohexadiene, or dihapto-complexed mono-deuterated cyclohexadiene ligands.

FIG. 3C is a schematic drawing showing the synthesis of isotopologues of cyclohexene comprising 3 deuterium atoms (i.e., d₃ isotopologues) from tungsten complexes comprising dihapto-complexed, partially deuterated cyclohexadiene or allyl ligands.

FIG. 3D is a schematic drawing showing the synthesis of isotopologues of cyclohexene comprising 6, 7, or 8 deuterium atoms (i.e., d₆, d₇, or d₈ isotopologues) from tungsten complexes comprising dihapto-complexed, partially deuterated allyl ligands.

FIG. 3E is a schematic drawing showing isotopomers of cyclohexene that can be prepared according to the presently disclosed method from tungsten complexes of benzene, 1,4-cyclohexadiene, benzene-d₈ and 1,4-cyclohexadiene-d₈.

FIG. 3F is a schematic drawing of (R)-tungsten trispyrazolylborate nitrosomethyl trimethylphosphine dihapto-coordinated cyclohexene ((R)-9) and a graph showing the expected proton (¹H) nuclear magnetic resonance (NMR) signal intensities of the different enantiotopomers of (R)-9.

FIG. 4A is a schematic drawing showing exemplary synthetic pathways to isotopologues of a 3-(trifluoromethyl)cyclohex-1-ene tungsten complex.

FIG. 4B is a schematic diagram showing exemplary synthetic pathways to isotopologues of a 3-cyanocyclohex-1-ene tungsten complex.

FIG. 4C is a schematic drawing showing exemplary synthetic pathways to functionalized isotopologues and stereoisotopologues of a 3-(trifluoromethyl)cyclohex-1-ene tungsten complex and of a 3-cyanocyclohex-1-ene tungsten complex summarized from FIGS. 4A and 4B and the further synthetic elaboration of these complexes in the to provide exemplary functionalized cyclohexane isotopologues.

FIG. 4D is a schematic drawing showing exemplary chemo- and stereoselectively deuterated cyclohexene complexes prepared according to the presently disclosed method and comprising trifluoromethyl (CF₃) and cyano (CN) substituents.

FIG. 5A is a schematic drawing showing exemplary synthetic pathways to isotopologues of tetrahydropyridine (THP) according to the presently disclosed methods.

FIG. 5B is a schematic drawing showing exemplary synthetic pathways to isotoplogues of tetrahydropyridines (THPs) related to methylphenidate and structures of possible stereoisotopomers of methylphenidate (177) and related trifluoromethyl-substituted compounds (178 and 179).

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. Although any methods, devices, and materials similar or equivalent to those described herein can be used in the practice or testing of the presently disclosed subject matter, representative methods, devices, and materials are now described. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a metal ion” includes a plurality of such metal ions, and so forth.

Unless otherwise indicated, all numbers expressing quantities of size, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size (i.e., diameter), weight, concentration or percentage is meant to encompass variations of in one example ±20% or +10%, in another example ±5%, in another example ±1%, and in still another example ±0.1% from the specified amount, as such variations are appropriate to perform the disclosed methods.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein the term “alkyl” can refer to C₁-20 inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In some embodiments, “alkyl” refers, in particular, to C₁₋₈ straight-chain or branched chain unsaturated alkyls (e.g., methyl, ethyl, n-propy, isopropyl, n-butyl, sec-butyl, iso-butyl, tert-butyl, pentyl, hexyl, heptyl, and octyl.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. In some embodiments, there can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Heteroaryl” as used herein refers to an aryl group that contains one or more non-carbon atoms (e.g., O, N, S, Se, etc) in the backbone of a ring structure. Nitrogen-containing heteroaryl moieties include, but are not limited to, pyridine, imidazole, benzimidazole, pyrazole, pyrazine, triazine, pyrimidine, and the like.

“Aralkyl” refers to an -alkyl-aryl group, optionally wherein the alkyl and/or aryl moiety is substituted.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “arylene” refers to a bivalent aromatic group, e.g., a bivalent phenyl or napthyl group. The arylene group can optionally be substituted with one or more aryl group substituents and/or include one or more heteroatoms.

The term “alkenyl” as used herein refers to a compound comprising one or more carbon-carbon double bond.

The term “amino” refers to the group —N(R)₂ wherein each R is independently H, alkyl, substituted alkyl, aryl, substituted aryl, aralkyl, or substituted aralkyl. The terms “aminoalkyl” and “alkylamino” can refer to the group —N(R)₂ wherein each R is H, alkyl or substituted alkyl, and wherein at least one R is alkyl or substituted alkyl. “Arylamine” and “aminoaryl” refer to the group —N(R)₂ wherein each R is H, aryl, or substituted aryl, and wherein at least one R is aryl or substituted aryl, e.g., aniline (i.e., —NHC₆H₅).

The terms “primary amine” and “secondary amine” as used herein refer to compound having the structure HN(R)₂ wherein, for the primary amine, one R is H and one R is alkyl, substituted alkyl, aryl, substituted aryl, aralkyl, or substituted aralkyl; and for the secondary amine, both R are independently selected from alkyl, substituted alkyl, aryl, substituted aryl, aralkyl, or substituted aralkyl.

The term “thioalkyl” can refer to the group —SR, wherein R is selected from H, alkyl, substituted alkyl, aralkyl, substituted aralkyl, aryl, and substituted aryl. Similarly, the terms “thioaralkyl” and “thioaryl” refer to —SR groups wherein R is aralkyl and aryl, respectively.

The terms “halo”, “halide”, or “halogen” as used herein refer to fluoro, chloro, bromo, and iodo groups.

The term “perfluoro”, e.g., as used in “perfluoralkyl” refers to a group or compound wherein all the hydrogens have been replaced by F. An exemplary perfluoroalkyl group is trifluoromethyl, i.e., —CF₃.

The term “hydroxyl” refers to the —OH group.

The terms “mercapto” or “thiol” refer to the —SH group.

The terms “carboxylate” and “carboxylic acid” can refer to the groups —C(═O)O— and —C(═O)OH, respectively. In some embodiments, “carboxylate” can refer to either the —C(═O)O— or —C(═O)OH group.

The terms “sulfonyl”, “sulfone”, and “sulphone” as used herein refer to the —S(═O)₂R group, wherein R is alkyl, substituted alkyl, aralkyl, substituted aralkyl, aryl, or substituted aryl.

The terms “sulfonamide”, “sulphonamide”, “sulfonamidyl,” and “sulfamyl” refer to the —S(═O)₂(R)₂ group wherein each R is independently H, alkyl, substituted alkyl, aralkyl, substituted aralkyl, aryl, or substituted aryl.

The term “phosphine” refers to a group or compound the formula PR3, wherein each R is independently alkyl, aralkyl, or aryl.

The term “silyl” refers to groups comprising silicon atoms (Si).

The term “acyl” as used herein refers to a group —C(═O)R, wherein R is alkyl, substituted alkyl, aralkyl, substituted aralkyl, aryl, or substituted aryl. “Ac” refers to an acyl group where R is CH₃.

A line crossed by a wavy line, e.g., in the structure:

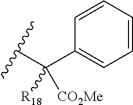

indicates the site where the indicated substituent can bond to another group. A wavy line used as a bond in a chemical structure (e.g., between the benzylic carbon and R₁₈ in the structure above) can also represent unspecified stereochemistry of the bond, wherein the compound can be a single stereoisomer or a mixture of the two possible stereoisomers.

A dashed line representing a bond in a chemical formula indicates that the bond can be either present or absent. For example, the chemical structure:

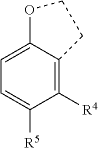

refers to compounds wherein oxygen can be bonded to a methyl or ethyl group or where the oxygen can be part of a ring fused to the aryl ring.

The term “arene” refers to an aromatic group or compound. The term “arene” refers to an unsubstituted or substituted aromatic carbocyclic moiety that is planar and comprises 4n+2 pi (π) electrons, according to Huckel's Rule, wherein n=1, 2, or 3, as commonly understood in the art. The term “arene” includes monocyclic and polycyclic aromatics and generally contains from, for example, 6 to 30 carbon atoms (e.g., from 6 to 18 carbons, from 6 to 14 carbons, or from 6 to 10 carbons). Non-limiting examples of arenes include benzene, naphthalene, anthracene and pyrene.

The term “alicyclic” as used herein refers to a nonaromatic group or compound comprising 1 or more carbon atom rings (including fused, bridging and spiro-fused rings). Alicyclic compounds can be saturated or unsaturated (e.g., can comprise one or more carbon-carbon double or triple bonds). In some embodiments, the alicyclic compound comprises one or more carbon rings comprising (exclusive of any alkyl group substituents) 3 to 20 carbon atoms (e.g., 3, 4, 5, 6, 7, 8, 9, 10, 11, 12, 13, 14, 15, 16, 17, 18, 19 or 20 carbon atoms). Exemplary alicyclic compounds include, for example, cyclopropane, cyclobutene, cyclopentane, cyclopentadiene, cyclohexane, cyclohexene, cyclohexadiene, cycloheptane, cyclohepene, cycloheptadiene, cycloheptatriene, and cyclooctene.

A heteroalicyclic compound is an alicyclic compound or group as defined above which has, in addition to carbon atoms, one or more ring heteroatoms (e.g., O, S, N, P and Si). In some embodiments, the heteroalicyclic compound or group preferably contain from one to four heteroatoms (i.e., 1, 2, 3, or 4 heteroatoms), which can be the same or different. Exemplary heterocyclic compounds include dihydrofuran, dihydrothiophene, dihydropyrrole, piperidine, and tetrahydropyridine.

The term “nucleophile” refers to a molecule or ion that can form a bond with an electron deficient group (or electrophile, e.g., a carbonyl carbon) by donating one or two electrons. Nucleophiles include, but are not limited to, carbon, oxygen, and sulfur nucleophiles. The term nucleophile as used herein also includes reagents that can deliver a hydride (e.g., NaBH₄) or deuteride. Exemplary nucleophiles include, water, hydroxide, hydrides, cyanide salts, alcohols (i.e., aromatic and aliphatic alcohols), alkoxides, aryloxides (e.g., phenoxides), thiols (e.g, HS-alkyl, HS-aryl), thiolates (e.g., —S-alkyl and —S-aryl), enolates (e.g., protected enolates or enolate salts, such as lithium or trialkylsilyl enolates), organozinc compounds (e.g., dialkyl zinc compounds), alkyl and aryl magnesium halides (i.e., Grignard reagents), alkynides (—C≡CR, wherein R is alkyl, such as acetylide salts or substituted acetylide salts), phosphines (e.g., trialkylphosphines, such as trimethylphosphine), and amines (e.g., ammonia, primary amines, and secondary amines). Nucleophiles can also be provided as salts, such as, but not limited to, alkali metal salts (i.e., salts comprising an anionic nucleophile, such as an alkoxide, aryloxide, or thiolate, and an alkali metal cation, such as but not limited to a sodium (Na), potassium (K), lithium (Li), calcium (Ca), or cesium (Cs) cation.

The term “aprotic solvent” refers to a solvent molecule which can neither accept nor donate a proton. Examples of aprotic solvents include, but are not limited to, esters, such as ethyl acetate; carbon disulphide; ethers, such as, diethyl ether, tetrahydrofuran (THF), ethylene glycol dimethyl ether, 1,4-dioxane, dimethoxyethane, dibutyl ether, diphenyl ether, MTBE, and the like; aliphatic hydrocarbons, such as hexane, pentane, cyclohexane, and the like; aromatic hydrocarbons, such as benzene, toluene, naphthalene, anisole, xylene, mesitylene, and the like; and symmetrical halogenated hydrocarbons, such as carbon tetrachloride, tetrachloroethane, and dichloromethane. Additional aprotic solvents include, for example, acetone; butanone; nitriles (e.g., acetonitrile or butyronitrile), chlorobenzene, chloroform, 1,2-dichloroethane, dimethylacetamide, N,N-dimethylformamide (DMF), and dimethylsulfoxide (DMSO).

The term “protic solvent” refers to a solvent molecule which contains a hydrogen atom bonded to an electronegative atom, such as an oxygen atom or a nitrogen atom. Typical protic solvents include, but are not limited to, carboxylic acids, such as acetic acid, alcohols, such as methanol and ethanol, amines, amides, and water.

The term “Bronsted acid” as used herein refers to a compound that can donate a proton. For example, these acids release protons to a corresponding base. Non-limiting Bronsted acids are acetic acid (CH₃COOH), sulfuric acid (H₂SO₄), para-toluenesulfonic acid (TsOH), ammonium salts (e.g., diphenylammonium triflate (DPhAT)), trifluoromethanesulfonic acid (triflic acid; HOTf); methanesulfonic acid (MsOH), hexafluorophosphoric acid (HPF₆), tetrafluoroboric acid (HBF₄), hydrochloric acid (HCl), and hydrobromic acid (HBr). The term “strong acid” refers to an acid that completely dissociates in aqueous solution. In some embodiments, the strong acid has a pKa of <−1.74.

As used herein, the term “isotope” refers to one of two or more variants of an atom of an element that have the same number of protons (i.e., the same atomic number), but different numbers of neutrons. For example, hydrogen has three naturally occuring isotopes: protium (¹H), which contains one proton but no neutrons; deuterium (D or ²H), which has one proton and one neutron; and tritium (T or ³H) which has one proton and two neutrons. Protium and deuterium are both stable, while tritium is radioactive, with a half-life of about 12.32 years. Protium is by far the most naturally abundant of the three naturally occurring hydrogen isotopes (i.e., 99.98% compared to 0.02% D and a trace of T). Unless otherwise stated, when a position is designated specifically as “H” or “hydrogen,” the position is understood to have hydrogen at its natural isotopic composition. Compounds and atoms containing their natural isotopic composition can also be referred to herein as “non-enriched” or “non-isotopically enriched” compounds or atoms. The terms “isotopically enriched” and “isotopic” as used herein, and unless otherwise specified, can refer to an atom having an isotopic composition other than the natural isotopic composition of that atom. “Isotopically enriched” can also refer to a compound containing at least one atom having an isotopic composition other than the natural isotopic composition of that atom.

As used herein, the term “isotopologue” as used herein refers generally to molecules that have the same elemental composition and bonding arrangement, but which differ in isotopic composition. Each isotopologue has a unique exact mass, but not an unique structure.

The term “isotopomer” as used herein refers to compound that has the same number of isotopic atoms as another compound, but that is a constitutional isomer or stereoisomer of the other compound based on the location of one or more isotopic atoms. The term “stereoisotopomer” refers to a compound that is an isotopomer that is a stereoisomer of another compound.

A “coordination complex” or “metal complex” as used herein refers to a compound in which there is a coordinate bond between a metal ion and an electron pair donor, ligand or chelating group. Thus, ligands or chelating groups are generally electron pair donors, molecules or molecular ions having unshared electron pairs or pi (π) electrons available for donation to a metal ion.

The term “coordinate bond” refers to an interaction between an electron pair donor and a coordination site on a metal ion resulting in an attractive force between the electron pair donor and the metal ion. The use of this term is not intended to be limiting, in so much as certain coordinate bonds also can be classified as have more or less covalent character (if not entirely covalent character) depending on the characteristics of the metal ion and the electron pair donor.

As used herein, the term “ligand” refers generally to a species, such as a molecule or ion, which interacts, e.g., binds, in some way with another species. More particularly, as used herein, a “ligand” can refer to a molecule or ion that binds a metal ion in solution to form a “coordination complex.” See Martell, A. E., and Hancock, R. D., Metal Complexes in Aqueous Solutions, Plenum: New York (1996), which is incorporated herein by reference in its entirety.

The term “hapacity” as used herein refers to the number of contiguous atoms in a metal ligand that are coordinated to a metal center. The Greek letter eta (q) can be used with a superscripted number to indicate the hapacity of a metal ligand.

### II. Methods of Preparing Isotopologues and Stereoisotopomers

Processes for incorporating hydrogen isotopes (i.e., D and T) into organic compounds are often unselective and can provide products where the stereoisotopic purity can be difficult to measure.⁷⁻⁸ For example, previous methods for deuterating benzene to provide isotopically labelled cyclohexadienes and cyclohexenes lead to over-reduction and mixtures of isotopologues. For instance, typically, hydrogenation of benzene using D₂ gas leads to isotopologue mixtures of cyclohexane rather than cyclohexene.¹⁰⁻¹² Thus, although Taube et al. demonstrated that the complex [Os(NH₃)₅(η²-benzene)]²⁺ could be deuterated to form a single stereoisotopomer of [Os(NH₃)₅(η²-cyclohexene-d₄)]²⁺, i.e., (3R,4R,5S,6S)-cyclohex-1-ene-3,4,5,6-d₄), using D₂ and a Pd/C catalyst,¹³ such results represent a rare exception. To date, methods of providing discrete isotopologues and stereoisotopomers of cyclohexene have been largely lacking in the literature.

Described herein is a new approach toward the preparation of stereoselectively isotopically labelled “building blocks” for pharmaceutical research. As described further in the Examples, provided herein is a proof of concept through a four-step conversion of benzene to cyclohexene, as bound to a transition metal complex. Using different combinations of deuterated and proteated acid and hydride reagents, positions of deuterium incorporation can be precisely controlled on the cyclohexene ring. In total, based on the presently disclosed method, 52 unique stereoisotopomers of cyclohexene are available, in the form of ten different isotopologues. This concept was further extended to prepare discrete stereoisotopomers of functionalized cyclohexenes that can be incorporated into drug design. Preparation of discrete stereoisotopomers of nitrogen-containing compounds (e.g., tetrahydropyridines and piperidines) from pyridine complexes is also described.

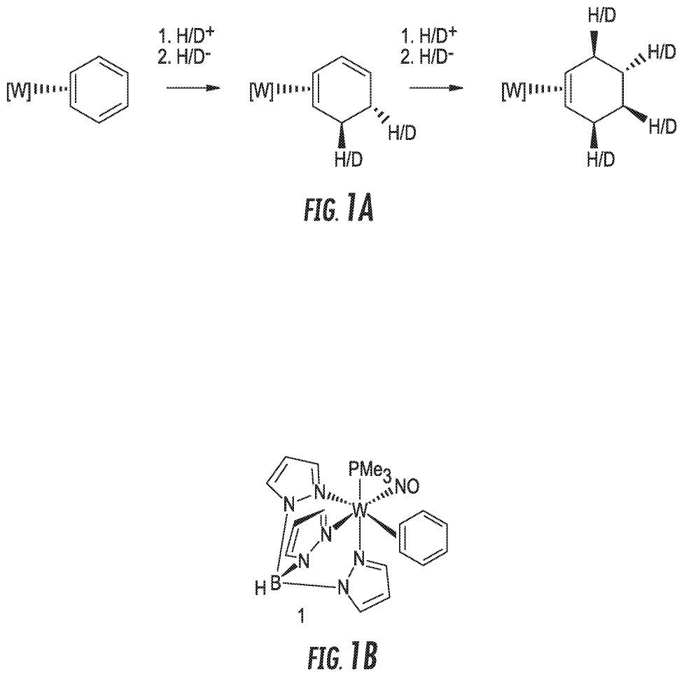

Accordingly, in some embodiments, the presently disclosed subject matter relates to the reduction of dihapto-coordinated arenes, heteroarenes, and alicyclic polyalkenyl compounds (e.g., alicyclic dienes and trienes) in a step-wise manner by sequential regio- and/or stereo additions of “ionic hydrogen” (H⁺ and H⁻), “ionic deuterium” (D⁺ and D⁻), and/or “ionic tritium” (T⁺ and T⁻). For example, in one aspect, the presently disclosed subject matter provides a method where a dihapto-coordinated benzene is converted to cyclohexene using four well-defined additions of ionic hydrogen and/or ionic deuterium through an η²-1,3-cyclohexadiene intermediate. See FIG. 1A. As these reactions can be performed regio- and stereoselectively, the method provides access to a diverse set of isotopologues and stereoisotopomers of cyclohexene using various combinations of proteated, deuterated, or tritiated reagents. In some embodiments, an ionic hydrogen, ionic deuterium, or ionic tritium addition can be replaced by the addition of a polyatomic nucleophile or, when the dihapto-coordinated arene is anisole, by the addition of an arene or heteroarene (e.g., phenol, anisole, carbazole, estradiol, thiophene, or furan).

In some embodiments, the presently disclosed subject matter provides a method of preparing an isotopologue or a stereoisotopomer of a cyclic or heterocyclic alkene or diene, the method comprising: (a) providing a first metal complex comprising a transition metal and a dihapto-coordinated ligand selected from an arene, a heteroarene or salt thereof (e.g., a pyridinium salt), and an alicyclic compound comprising at least two alkene groups (i.e., at least two carbon-carbon double bonds); (b) reducing the dihapto-coordinated ligand, thereby forming a second metal complex comprising the transition metal and a dihapto-coordinated cyclic or heterocyclic alkene or diene ligand; and (c) decomplexing the dihapto-coordinated cyclic or heterocyclic alkene or diene ligand from the transition metal (e.g. by treatment with an oxidant), thereby providing the isotopologue or stereoisotopomer of the cyclic or heterocyclic alkene or diene, wherein said isotopologue or stereoisotopomer is isotopically enriched (i.e., contains at least one deuterium or tritium).

In some embodiments, the transition metal is selected from tungsten (W), rhenium (Re), osmium (Os), and molybdenum (Mo) and the first metal complex is prepared by complexing the arene, heteroarene, or alicyclic compound to a W, Re, Os, or Mo metal complex, such as a complex known in the art as a dearomatization agent and/or that is known in the art to bind aromatic molecules in a dihapto fashion. Dihapto-coordination can activate the uncoordinated portion of the η²-bound system through π-donation, while at the same time protecting the coordinated double bond.⁹ In some embodiments, the dearomatization agent is a saturated (18 electron), octahedral W, Re, Os, or Mo complex, such as a pentaammineosmium(II) complex, a rhenium trispyrazolylborate (Tp) carbonyl (CO)N-methyl imidazole (Melm) complex, a MoTp nitroso (NO) 4-(dimethylamino)pyridine (DMAP) complex, a WTp(NO) trialkylphosphine complex, or the salts thereof. Thus, in some embodiments, the first metal complex can be selected from the group including, but not limited to [Os(NH₃)₅(η²-benzene)]²⁺, ReTp(CO(Melm)(η²-benzene), MoTp(NO)(DMAP)(η²-benzene) and WTp(NO)(trialkylphosphine)(η²-benzene) and complexes prepared by exchange of the benzene ligand of Os(NH₃)₅(η²-benzene)]²⁺, ReTp(CO(Melm)(η²-benzene), MoTp(NO)(DMAP)(η²-benzene) and WTp(NO)(trialkylphosphine)(η²-benzene) with other arenes, heteroarenes or alicyclic polyalkenes. In some embodiments, the alkyl group of the phosphine ligand of the W complex is methyl (Me) or butyl (Bu).

In some embodiments, the transition metal is W. Thus, in some embodiments, providing the first metal complex comprises contacting a WTp(NO)(trialkylphosphine)(η²-benzene) with an arene or an alicyclic compound comprising at least two carbon-carbon double bonds, and forming the first metal complex via ligand exchange. In some embodiments, the first metal complex is a WTp(NO)(trialkylphosphine)(η²-arene), a WTp(NO)(trialkylphosphine)(η²-diene) or a WTp(NO)(trialkylphosphine)(η²-triene). In some embodiments, the first metal complex is a WTp(NO)(trimethylphosphine)(η²-arene), a WTp(NO)(trimethylphosphine)(η²-diene) or a WTp(NO)(trimethylphosphine)(η²-triene). In some embodiments, providing the first metal complex comprises contacting (WTp(NO)(PMe₃)(η²-benzene)) with an arene or an alicyclic compound comprising at least two carbon-carbon double bonds, thereby forming WTp(NO)(PMe₃)(η²-arene), a WTp(NO)(PMe₃)(η²-diene) or a WTp(NO)(PMe₃)(η²-triene) (via ligand exchange). Ligand exchange can be performed, for example, in an ether solvent such as dimethyl ether, DME, or THF at room temperature using a molar excess (e.g., at least a four fold molar excess) of the ligand which is exchanging with the benzene. When the ligand replacing the benzene is a liquid (e.g., benzene-d₆), the contacting can also be performed neat (i.e., without an additional solvent).

Alternatively, in some embodiments, providing the first metal complex can comprise contacting a tungsten tripyrazolylborate nitroso trialkylphosphine halide complex (e.g., WTp(NO)(PMe₃)Br) with an arene (e.g. benzene) in the presence of an alkali metal (e.g., sodium (Na), lithium (Li), or potassium (K)) thereby forming a WTp(NO)(trialkylphosphine)(η²-arene) complex (e.g., a WTp(NO(PMe₃)(η²-arene) complex). In some embodiments, the alkali metal is Na. In some embodiments, the contacting is performed under oxygen free conditions. In some embodiments, the contacting can be performed at room temperature for several hours (e.g., 8 hours or more).

As another alternative, providing the first metal complex can comprise contacting a WTp(NO)(trialkylphosphine)(η²-benzene) complex (e.g., WTp(NO(PMe₃)(η²-benzene)) with a pyridine borane (e.g., pyridine-borane or a substituted pyridine-borane) to form a WTp(NO)(trialkylphosphine)(η²-pyridine-borane) (e.g., WTp(NO)(PMe₃)(η²-pyridine-borane)). The pyridine-borane complex can then be contacted with a Bronsted acid (e.g., diphenylammonium triflate (DPhAT)) to remove the borane and provide a WTp(NO)(trialkylphosphine)(η²-pyridium) salt. In some embodiments, the WTp(NO)(trialkylphosphine)(η²-pyridium) salt is a WTp(NO)(PMe₃)(η²-pyridium) salt, such as a WTp(NO)(PMe₃)(η²-pyridinium) triflate (OTf), halide or other salt. In some embodiments, the WTp(NO)(PMe₃)(η²-pyridinium) salt or other WTp(NO)(trialkylphosphine)(η²-pyridinium) salt can be contacted with an anhydride (e.g., acetic anhydride or p-toluenesulfonic anhydride) or acid chloride in the presence of a weak base, such as a sterically hindered pyridine like 2-6-ditertbutylpyridine (DTBP), to provide a WTp(NO)(trialkylphosphine)(η²-N-acylated pyridinium) salt or a WTp(NO)(trialkylphosphine)(η²-N-sulfonated pyridinium) salt, e.g., a WTp(NO)(PMe₃)(η²-N-acylated pyridinium) salt or a WTp(NO)(PMe₃)(η²-N-tosylated pyridinium) salt.

In some embodiments, the dihapto-coordinated ligand of the first metal complex is selected from the group including, but not limited to, benzene, naphthalene, anthracene, cyclopentadiene, cyclohexadiene, furan, 2,3-dihdyrobenzofuran, indole, anisole, pyrrole, N-sulfonated pyrrole, pyridine, deuterated, tritiated, and/or substituted analogues thereof, and salts thereof (e.g., pyridinium, N-acylated pyridinium, and N-sulfonated pyridinium salts). For example, suitable mono-substituted benzenes and naphthalenes that can be used in the presently disclosed method include benzenes substituted with an electron withdrawing group, such as, but not limited to, alkyl (e.g., methyl), perfluoroalkyl (e.g., perfluoromethyl), cyano, pentafluorothio (—SF₅), sulfonyl (e.g., —SO₂-aryl groups, such as —SO₂-phenyl), and sulfonamide (—SO₂—NR₂, wherein each R is independently alkyl, aralkyl or aryl). Suitable isotopically labelled ligands include perdeuterated compounds, such as benzene-d₆, toluene-d₈, pyridinium-d₅ salts, N-acylated pyridinium-d₅ salts, and N-sulfonated pyridinium-d₅ salts or pertritiated compounds.

In some embodiments, the dihapto-coordinated ligand is a mono- or di-substituted 2,3-dihdyrobenzofuran, wherein the 2,3-dihydrobenzofuran is substituted at one or more of the carbons of the aromatic ring with substituents independently selected from alkyl, aryl, and perfluoroalkyl (e.g., —CF₃). In some embodiments, the di-hapto-coordinated ligand is a substituted pyridine or pyridinium salt wherein the pyridine is substituted ortho or meta to the nitrogen atom with a substituent selected from alkyl, perfluoroalkyl, —CF₂-alkyl, —CF₂-aryl, aralkyl, aryl (e.g., phenyl) and heteroaryl (e.g., pyridyl). In some embodiments, the dihapto-coordinated ligand is selected from the group consisting of benzene, substituted benzene, naphthalene, substituted naphthalene, a pyridinium salt, a substituted pyridinium salt and deuterated or tritiated analogues thereof.

In some embodiments, the reducing of step (b) comprises contacting the first metal complex sequentially with at least a first reagent and a second reagent. In some embodiments, the first reagent is a Bronsted acid or a deuterated or tritiated analogue thereof, and the second reagent is a nucleophilic reagent. Contacting with the first reagent can thus add one hydrogen, deuterium or tritium atom to the dihapto-coordinated ligand while contacting with the second reagent adds a nucleophile. In some embodiments, the first reagent is a Bronsted acid or a deuterated analogue thereof. The additions can be both regio- and stereoselective. In some embodiments, the second reagent can be a hydride (H⁻), deuteride (D⁻), or tritium hydride (T⁻). Thus, in some embodiments, contacting with the second reagent also adds a hydrogen, deuterium or tritium atom to the coordinated ligand.

Suitable first reagents include strong acids such as, but not limited to, diphenylammonium triflate (DPhAT), trifluoromethanesulfonic acid (HOTf); sulfuric acid (H₂SO₄), hexafluorophosphoric acid (HPF₆), tetrafluoroboric acid (HBF₄), hydrochloric acid (HCl), and hydrobromic acid (HBr), and their deuterated and tritiated analogues (e.g., diphenyl ammonium-d₂ triflate (DPhAT-d₂) and deuterated trifluoromethanesulfonic acid (DOTf). In some embodiments, the first reagent is a tritiated acid, such as tritiated trifluoromethanesulfonic acid (TOTf), which can be prepared by making a solution of HOTf (e.g., 0.01M HOTf) in tritiated water (i.e., tritium oxide or “super heavy water”, T₂O). The contacting with the first reagent can be performed in an ether (e.g., diethyl ether, diglyme, 1,2-dimethoxyethane (DME), or methyl-t-butyl ether), nitrile (e.g., acetonitrile) or ester (e.g., ethyl acetate) solvent. Reactions involving the metal complexes described herein are typically performed at temperatures below about −20° C. (e.g., temperatures between about −97° C. (i.e., the melting point of methanol, which can used as a solvent in steps) and about −20° C.) under an inert atmosphere (e.g., nitrogen or argon gas). In some embodiments, the temperature is between about −78° C. and about −20° C. In some embodiments, the contacting with the first reagent is performed at a temperature between about −60° and about −20° C. In some embodiments, the contacting is performed at a temperature of about 30° C. In some embodiments, the contacting is performed for a period of time ranging from about 5 minutes and about 15 minutes.

As noted above, in some embodiments, the second/nucleophilic reagent is a hydride, deuteride, or tritium hydride reagent. Suitable hydride, deuteride, and tritium hydride reagents that can be used according to the presently disclosed methods include, but are not limited to, sodium borohydride (NaBH₄), tetrabutylammonium borohydride (N(Bu)₄BH₄), sodium cyanoborohydride (NaBH₃CN), sodium trimethoxyborohydride (Na(MeO)₃BH), and lithium aluminum hydride (LAH) and their deuterium and tritium hydride counterparts. In some embodiments, the second reagent is a hydride or a deuteride reagent. In some embodiments, the second reagent is NaBH₄ and the contacting is performed in methanol. In some embodiments, the second reagent is sodium borodeuteride (NaBD₄) and the contacting is performed in deuterated methanol or a mixture of acetonitrile and a crown ether that is of a suitable size to complex Na, such as 15-crown-5 ether. In some embodiments, the contacting with the second reagent is performed at a temperature between about −78° C. and about −20° C. or between about −60° C. and about −20° C. In some embodiments, the contacting is performed at about −60° C. In some embodiments, the contacting is performed for a period of time of about 1 hour.

In some embodiments, the second reagent is another nucleophilic reagent, such as, but not limited to, a Grignard reagent (e.g., an alkyl or aryl magnesium halide), a dialkylzinc (e.g., diethylzinc), an enolate (e.g., a trialkylsilyl enolate, a sodium enolate, or a lithium enolate, such as, but not limited to, (CH₃)₂C═C(OMe)(OSiMe₃) or (CO₂Me)C═C(OLi)(OMe)), an alkoxide salt (e.g., a sodium or potassium alkoxide, such as methoxide), a cyanide salt (e.g., sodium cyanide), a phosphine (e.g., PMe₃), a primary amine, a secondary amine, and an alkynide (e.g., a sodium alkynide). In some embodiments, the nucleophilic reagent is selected from a cyanide salt, an enolate, a primary amine, a secondary amine, or an alkoxide. In some embodiments, the nucleophilic reagent is a cyanide salt. In some embodiments, the contacting with the nucleophilic reagent can be performed in a nitrile (e.g., acetonitrile) or alcohol (e.g., methanol) solvent. In some embodiments, the contacting can be performed at a temperature between about −78° C. and about −30° C. In some embodiments, the contacting is performed for a period of time between about 1 hour and about 16 hours.

In some embodiments, the decomplexing of step (c) comprises exposing the second metal complex to a temperature above about 0° C. for a period of time (e.g., a temperature between about 0° C. and about 30° C.). In some embodiments, the decomplexation comprises contacting the second metal complex with an oxidant. In some embodiments, the oxidant is a one-electron oxidant. Suitable one-electron oxidants include, but are not limited to, 2,3-dichloro-5,6-dicyano-1,4-benzoquinone (DDQ), an iron (Fe) (III) compound, nitrosonium hexafluorophosphate (NOPF₆), a copper (Cu) (II) salt, silver (Ag) (I) salt, or another oxidant with a potential greater than about 0.5 Volts (V) versus a normal hydrogen electrode (NHE). In some embodiments, contacting the second metal complex with the oxidant is performed in a solvent such as, but not limited to, benzene, acetonitrile, acetone, ethyl acetate, dimethylformamide (DMF), methanol, and methylene chloride. In some embodiments, the contacting with the oxidant is performed at a temperature between about 0° C. and about 30° C. In some embodiments, the contacting is performed at about 25° C. In some embodiments, the contacting with the oxidant is performed for a period of time between about 8 and about 12 hours.

In some embodiments, e.g., when the first metal complex comprises a dihapto-coordinated arene or heteroarene, step (b) comprises: (b1) contacting the first metal complex sequentially with a first reagent and a second reagent, wherein the first reagent is a Bronsted acid or a deuterated or tritiated analogue thereof, and wherein the second reagent is a nucleophilic reagent, thereby forming an intermediate metal complex comprising a dihapto-coordinated cyclic or heterocyclic diene ligand; and (b2) contacting the intermediate metal complex comprising the dihapto-coordinated cyclic or heterocyclic diene ligand sequentially with a third reagent and a fourth reagent, optionally wherein the third reagent is a Bronsted acid or a deuterated or tritiated analogue thereof, and wherein the fourth reagent is a nucleophilic reagent; thereby forming the second metal complex, wherein said second metal complex comprises a dihapto-coordinated cyclic or heterocyclic alkene ligand.

In some embodiments, the first reagent and the third reagent are each a Bronsted acid (such as one of the Bronsted acids described above suitable for use as the first reagent) or a deuterated or tritiated analogue thereof. In some embodiments, the first and third reagents are independently selected from a Bronsted acid and a deuterated analogue thereof. In some embodiments, the first reagent and the third reagent are each independently a strong acid or a deuterated or tritiated analogue thereof, wherein said strong acid is selected from the group consisting of diphenylammonium triflate (DPhAT), trifluoromethanesulfonic acid (HOTf); sulfuric acid (H₂SO₄), hexafluorophosphoric acid (HPF₆), tetrafluoroboric acid (HBF₄), hydrochloric acid (HCl), and hydrobromic acid (HBr). The contacting with the first reagent in step (b1) and with the third reagent in step (b2) can be performed in an ether (e.g., diethyl ether, diglyme, 1,2-dimethoxyethane (DME), or methyl-t-butyl ether), nitrile (e.g., acetonitrile) or ester (e.g., ethyl acetate) solvent. In some embodiments, the contacting with first reagent in step (b1) and the contacting with the third reagent in step (b2) is performed in an ether, nitrile, or ester solvent at a temperature between about −60° C. and about −20° C., optionally at about −30° C. In some embodiments, the contacting with the first and/or the third reagent is performed at a temperature of about 30° C.

In some embodiments, one of the second and fourth reagents is a hydride, deuteride, or tritium hydride reagent, such as one of the hydride reagents described above with regard to the second reagent, or a deuteride or tritium hydride analogue thereof. In some embodiments, the second reagent and the fourth reagents are each independently selected from a hydride reagent and a deuteride reagent. When the fourth reagent is a hydride, deuteride or tritium hydride reagent, the contacting can be performed using the same solvents and temperatures as described hereinabove for use when the second reagent is a hydride, deuteride, or tritium hydride reagent. For example, in some embodiments, at least one of the second and the fourth reagent is a hydride or a deuteride reagent selected from sodium borohydride (NaBH₄) and sodium borodeuteride (NaBD₄), wherein when the at least one of the second and the fourth reagent is NaBH₄, the contacting with the at least one of the second and the fourth reagent is performed in methanol and wherein when the at least one of the second and the fourth reagent is NaBD₄, the contacting with the at least one of the second and the fourth reagent is performed in deuterated methanol or a mixture of acetonitrile and 15-crown-5 ether. In some embodiments, the contacting with the at least one of the second and the fourth reagent is performed at a temperature between about −60° C. and about −20° C. In some embodiments, the contacting is performed at about −60° C.

In some embodiments, one of (or both of) the second and the fourth reagents is a nucleophilic reagent other than a hydride, deuteride, or tritium hydride, such as one of the other nucelophiles as described above with regard to the second reagent. Thus, in some embodiments, one of (or both of) the second and the fourth reagents is selected from the group comprising a cyanide salt, an alkoxide, an enolate, a phosphine, a Grignard reagent (i.e., an alkyl or aryl magnesium halide, such as an alkyl or aryl magnesium bromide), an alkynide (e.g., an alkynide salt, such as an acetylide salt), or a dialkylzinc. In some embodiments, one of the second and the fourth reagent is selected from the group consisting of a cyanide salt, an alkoxide salt, an enolate, a phosphine, a primary amine, and a secondary amine. In some embodiments, one of the second and fourth reagents is a cyanide salt (e.g., NaCN).

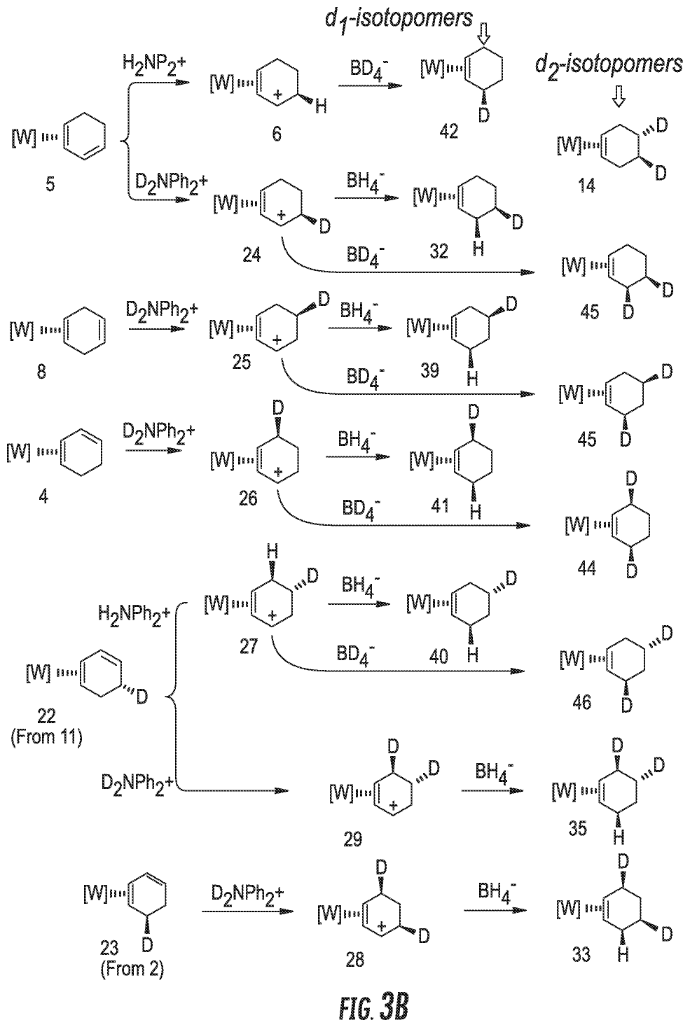

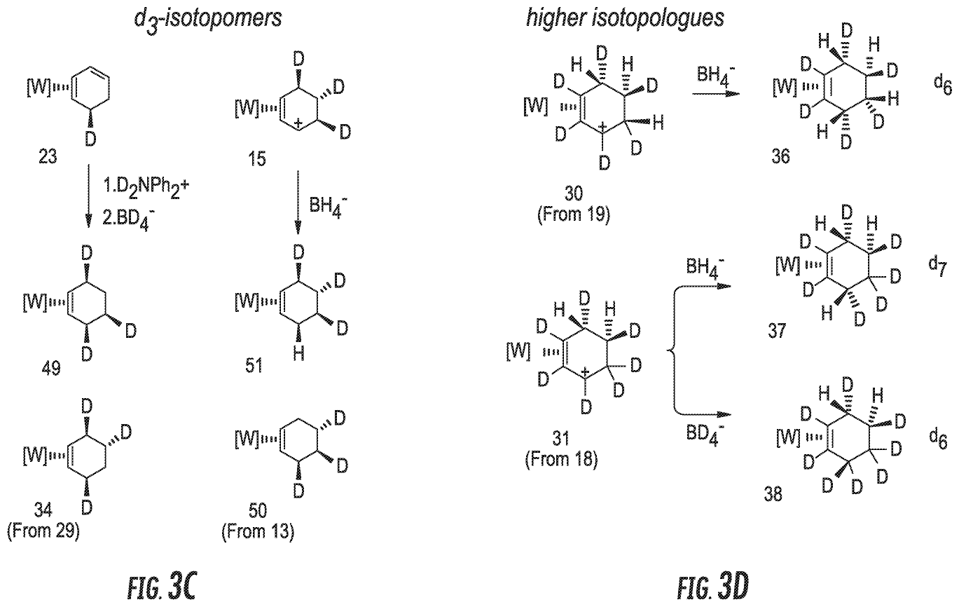

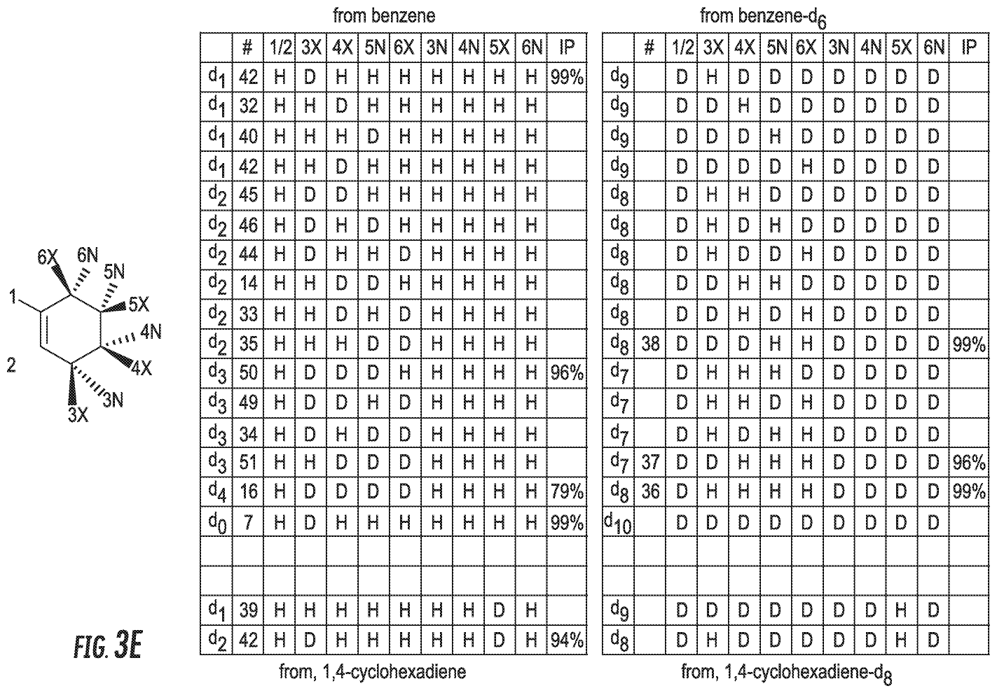

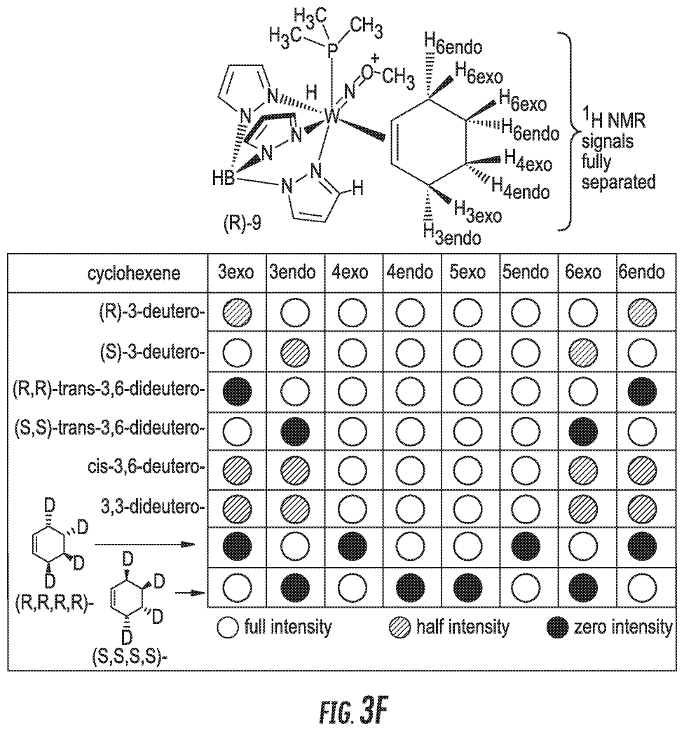

In some embodiments, at least one of steps (b1) and (b2) comprise a stereoselective addition of at least one of a proton (H⁺ or ¹H⁺), a deuteron (D⁺ or 2H⁺), a triton (T⁺ or ³H⁺), or a nucleophile, optionally the stereoselective addition of both a proton, deuteron or triton and a nucleophile. In some embodiments, the nucleophile is a hydride, a deuteride, or a tritium hydride. In some embodiments, the nucleophile is a hydride or a deuteride. See, e.g., FIGS. 3A-3D.

The presently disclosed method can provide access to isotopomers comprising at least one of at least two different isotopes of hydrogen. For example, the presently disclosed method can provide the addition of at least one D or T to a non-isotopically enriched arene, heteroarene or alicyclic polyalkenyl ligand (e.g., an alicyclic diene ligand). In some embodiments, the presently disclosed subject matter provides the addition of at least one H to an exhaustively deuterated or tritiated arene, heteroarene, or alicyclic polyalkenyl ligand. In some embodiments, the isotopic purity of the second metal complex and/or the decomplexed isotopologue or stereoisotopmer of a cyclic or heterocyclic alkene or diene is at least about 75% (e.g., where each isotopically enriched site has a minimum isotopic enrichment of at least about 75% or where at least about 75% of the complex or decomplexed isotopologue or stereoisotopomer has the same molecular mass, indicating the same isotopic composition). In some embodiments, the second metal complex and/or the decomplexed isotopologue or stereoisotopomer has about 80%, about 85%, about 90%, about 91%, about 92%, about 93%, about 94%, about 95%, about 96%, about 97%, about 98%, or about 99% isotopic purity.

In some embodiments, the dihapto-coordinated ligand of the first metal complex is an arene selected from benzene, a deuterated benzene, a substituted benzene, and a substituted deuterated benzene; and step (b1) comprises: (b1-i) contacting the first metal complex with an acid or a deuterated acid, thereby forming a metal complex comprising a dihapto-coordinated benzenium ligand; and (b1-ii) contacting the metal complex comprising the dihapto-coordinated benzenium ligand with a nucleophilic reagent, thereby forming a the intermediate metal complex, wherein said intermediate metal complex comprises a dihapto-coordinated cyclohexadiene ligand; wherein step (b2) comprises: (b2-i) contacting the intermediate metal complex with an acid or a deuterated acid, thereby forming a metal complex comprising a dihapto-coordinated allyl ligand; and (b2-ii) contacting the metal complex comprising the dihapto-coordinated allyl ligand with a nucleophilic reagent, thereby forming the second metal complex, wherein said second metal complex comprises the dihapto-coordinated cyclohexene ligand; and step (c) comprises decomplexing the dihapto-coordinated cyclohexene ligand (e.g., by contacting the second metal complex with an oxidant and/or by exposing the second metal complex to a temperature between about 0° C. and about 30° C.), thereby providing the isotopologue or stereoisotopomer of a cyclohexene, wherein said isotopologue or stereoisotopomer comprises at least one deuterium (substituted on a ring carbon) and at least one hydrogen (substituted on a ring carbon). In some embodiments, the isotopologue or stereoisotopomer of cyclohexene is provided with at least about 75%, 80%, 85%, 90%, 95%, 96%, 97%, 98%, or about 99% isotopic purity.

Mechanistic studies described hereinbelow starting with WTp(NO)(PMe₃)(η²-benzene) have determined that protonation/deuteration of dienes and hydride/deuteride addition to η²-allyl complexes are diastereospecific, with both H⁺/D⁺ and H⁻/D⁻ being delivered anti to the metal. The possible stereoisotopomers provided according to the presently disclosed method using WTp(NO(PMe₃)(η²-ligand) where the ligand is benzene, benzene-d₆, 1,4-cyclohexadiene, and 1,4-cyclohexadiene-d₈ are also summarized, for example, in FIG. 3E.

Accordingly, in some embodiments, one or more of steps (b1-i), (b1-ii), (b2-i), and (b2-ii) (i.e., one, two, three or all four of steps (b1-i), (b1-ii), (b2-i), and (b2-ii)) are stereoselective. For example, in some embodiments, the dihapto-coordinated ligand of the first metal complex is benzene or deuterated benzene (i.e., benzene-d₆) and the contacting of step (b1-i) comprises endo-selective protonation or deuteration of the benzene or deuterated benzene ligand. In some embodiments, the nucleophilic reagent of step (b1-i) is a hydride or a deuteride reagent and the contacting of step (b1-i) comprises exo-selective addition of a hydride or deuteride to the benzenium ligand. In some embodiments, the contacting of step (b2-i) comprises exo-selective protonation or deuteration of the cyclohexadiene ligand. In some embodiments, the nucleophilic reagent of step (b2-ii) is a hydride or a deuteride reagent and the contacting of step (b2-ii) comprises anti-selective addition of a hydride or deuteride to the allyl ligand relative to the metal (which, in this case, also results in exo-selective addition of the hydride or deuteride to the allyl ligand). In some embodiments, the presently disclosed method provides a stereoisotopomer of a cyclohexene with a stereoselectivity of 22:1 or more (e.g., about 40:1). In some embodiments, the presently disclosed method provides a stereoisotopomer of a cyclohexene having an enantiomeric excess (ee) of at least about 80%.

In some embodiments, the arene of step (a) is benzene or a substituted benzene and the isotopologue or stereoisotopomer is a di-, d₂-, d₃-, or d₄-cyclohexene. In some embodiments, the arene is benzene-d₆ and the isotopologue or stereoisotopomer is a d₆-, d₇-, or d₈-cyclohexene. In some embodiments, the arene is a substituted benzene. In some embodiments, the arene is benzene mono-substituted with an electron-withdrawing group. Scheme 1 below shows potential isotopologues of cyclohexene provided by the presently disclosed method when starting with a mono-substituted benzene as the ligand in the first metal complex. Each site indicated as H/D can be either H or D. Alternatively, when an exhaustively deuterated toluene is used as the ligand in the first metal complex, R can be CD₃ and each of the carbons of the remaining alkene bond can be substituted by D, instead of H. In some embodiments, the substituted benzene comprises a substituent selected from alkyl, perfluoroalkyl, cyano, a sulfone, and a sulfonamide.

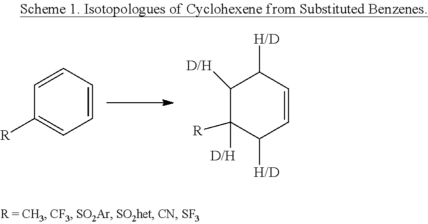

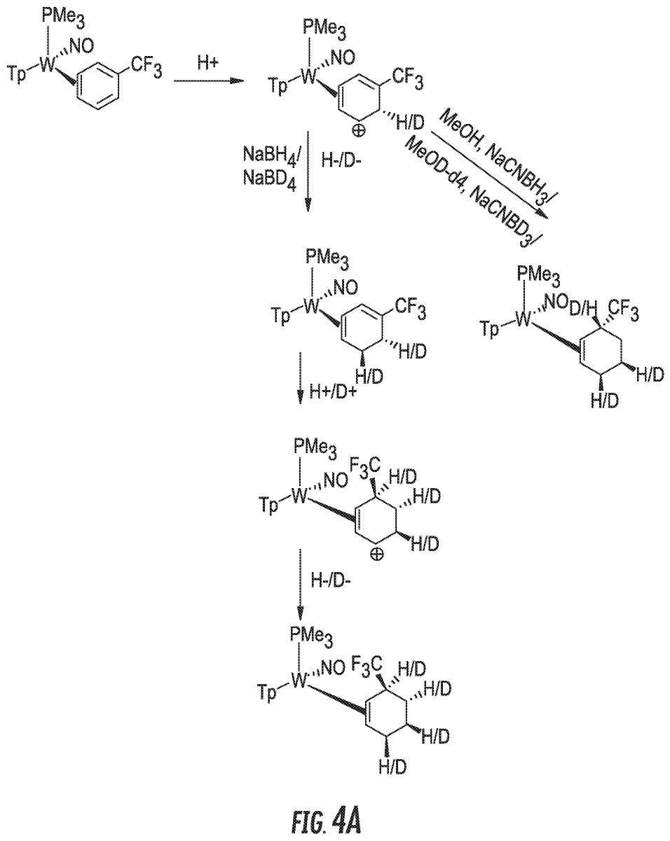

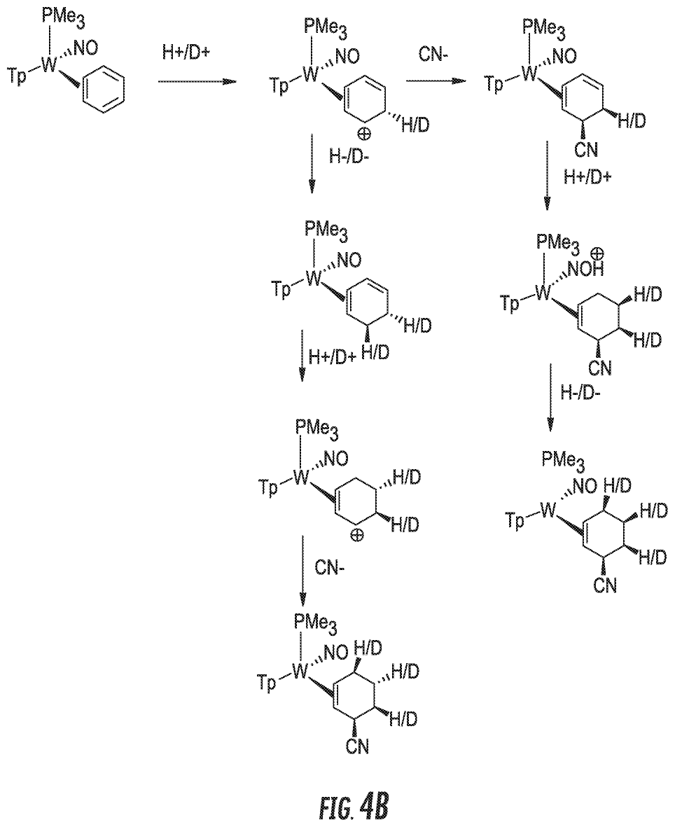

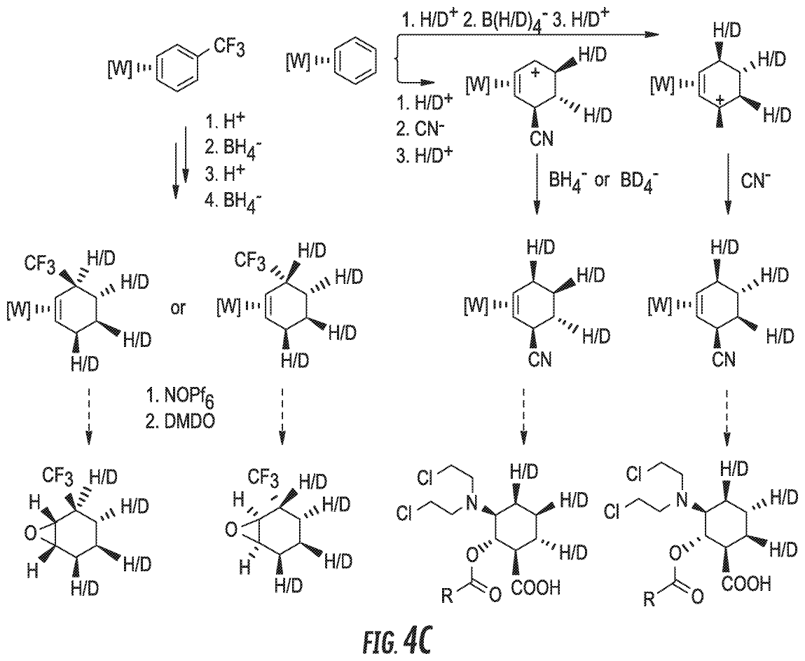

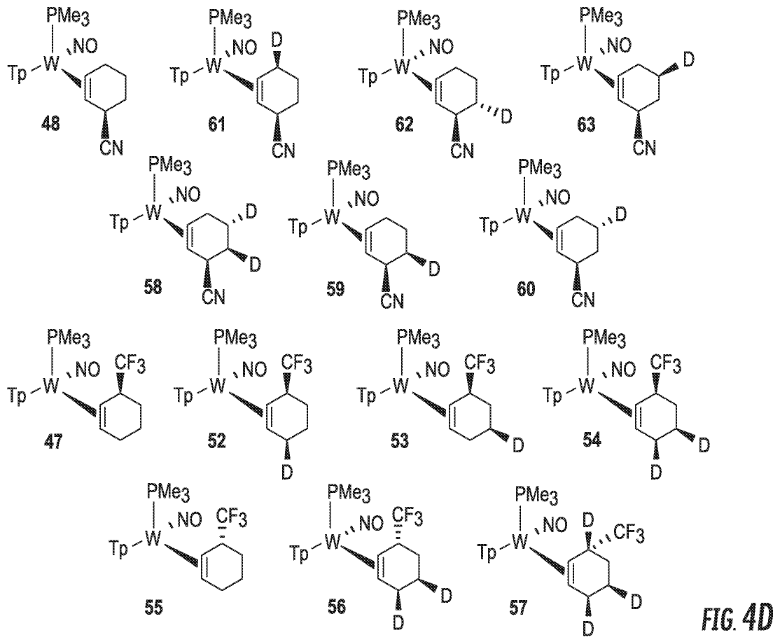

In some embodiments, the method further comprises contacting the isotopologue or stereoisotopomer of the cyclohexene with a dioxirane, optionally dimethyldioxirane (DMDO), thereby converting the isotopologue or stereoisoptopomer of the cyclohexene into an epoxide. See, for example, the left-hand side of FIG. 4C, which shows the decomplexation of a trifluoromethyl-substituted cycloalkene from a W complex using the oxidant NOPF₆, followed by epoxidation of the alkene using DMDO.

In some embodiments, the isotopologue or stereoisotopomer of the cyclic or heterocyclic alkene or diene is a synthetic intermediate of a deuterated active pharmaceutical ingredient (i.e., a deuterated version of a compound known or suspected of having a beneficial biological activity related to the treatment of a disease or a disease symptom). For example, as shown in the right side of FIG. 4C, a cyano-substituted cycloalkene prepared by the presently disclosed method can be decomplexed from the second metal complex (e.g., using an oxidant, such as NOPf₆) and then further reacted, e.g., to provide a deuterated form of a nitrogen mustard for use in cancer research. For example, 3-cyano-substituted cyclohexene can be subjected to a nine-step synthesis as previously described in the literature³⁰ involving the following reagents: (i) hydrochloric aci/hydrogen peroxide (HCl/H₂O₂); (ii) isobutylene, (iii) meta-chloroperbenzolic acid (mCPBA); (iv) sodium azide (NaN₃); (v) acetyl chloride; (vi) H₂/C; (vii) ethylene oxide; (viii) tosyl chloride (TsCl) and (ix) trifluoroacetic acid (TFA).

Isotopologues provided by the presently disclosed method when other arenes or alicyclic polyalkenes are used to prepare the first metal complex are shown in Schemes 2, 3, 4 and 5, below. For example, Scheme 2 shows the isotopologues/stereoisotopomers available when naphthalene, furan, or N-sulfonated pyrrole is the dihapto-coordinated ligand of the first metal complex in the presently disclosed method and treated stepwise with a Bronsted acid or a deuterated Bronsted acid (H/D⁺) and then with a hydride or deuteride (H/D⁻). See top three lines of Scheme 2. The decomplexed products are shown on the right side of the schemes and the pre-complexed starting material arene on the left. Scheme 2 (bottom line) also shows possible isotopologues/stereoisotopomers when cyclohexatriene is the dihapto-coordinated ligand in the first metal complex and treated stepwise, first to a hydride extractor such as trityl triflate (i.e., to provide a tropylium ion), then to a deuteride, and then to four additions of ionic hydrogen or deuterium.

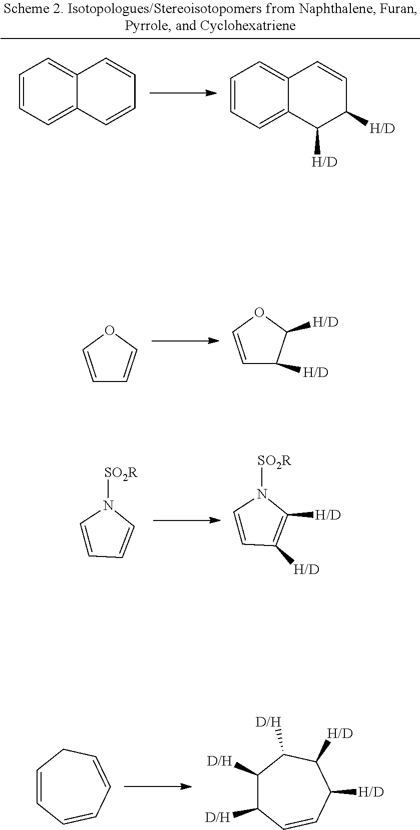

Scheme 3 shows the isotopologues provided when a disubstituted anisole or 2,3-dihydrobenzofuran (Scheme 3, left) is used as the dihapto-coordinated ligand in the first metal complex of the presently disclosed method and is subjected to four stepwise additions of ionic hydrogen and/or deuterium. The decomplexed products are shown on the right.

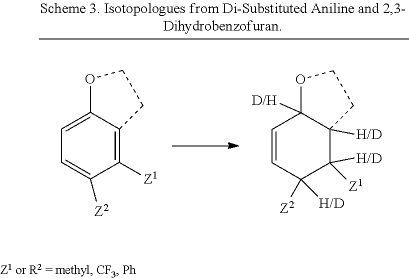

Scheme 4 shows the isotopologues provided when N-substituted indoline (e.g., N-aryl sulfone-substituted indoline) is used as the dihapto-coordinated ligand in the first metal complex of the presently disclosed method and is subjected to the stepwise addition of ionic hydrogen and/or deuterium. Possible R groups in Scheme 3 include, for example, Ts or another aryl sulfphone.

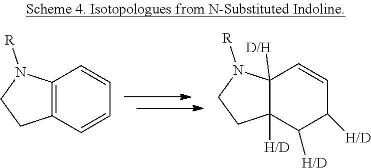

Scheme 5 shows the isotopologues provided when anisole or another alkoxybenzene is used as the dihapto-coordinated ligand of the first metal complex in the presently disclosed method. An alkoxybenzene (Scheme 5, top left, where R¹ is alkyl, e.g., methyl or ethyl) can be dihapto-coordinated in the first metal complex, treated to stepwise additions of four ionic hydrogen or deuterium, and decomplexed (e.g., via contact with an one electron oxidant) to provide an isotopologue of an alkoxy-substituted cyclohexene. See Scheme 5, top middle. In some embodiments, the alkoxy-substituted cyclohexene can be further reacted with a nucleophile (“R²—”), such as but not limited to a cyanide salt, an enolate, a Grignard reagent, an alkynide, or an alkyl lithium reagent, that can add to the alkoxy-substituted carbon, with the alkoxy group acting as a leaving group, resulting in the replacement of the alkoxy group with the nucleophile. See Scheme 5, top right. Alternatively, a first metal complex comprising the alkoxybenzene can be treated to double protonation (e.g., using triflic acid in dichloromethane at −60° C.), followed by addition of a neutral arene (R³H), such as phenol, anisole, carbazole, estradiol, thiophene or furan, to provide an oxonium intermediate (complexed to the metal of a transition metal complex, such as a W complex, although only the ligand is shown in the scheme). The oxonium can be reduced via treatment with a hydride reagent (e.g., NaBH₄). Subsequent addition of acid (e.g., triflic acid in methanol), eliminates methanol and generates an allyl intermediate, similar in structure to the allyl ligand intermediate prepared during the preparation of a cyclohexene from benzene according to the presently disclosed method The allyl intermediate can be contacted with nucleophile R² and decomplexed to provide the structure shown at the right side of the middle line in Scheme 5 below. Alternatively, the ketone products (see Scheme 5, bottom) can be prepared by hydrolysis of the oxonium intermediate with water.

The presently disclosed method can also be used for the regio- and stereospecific deuteration of various tetrahydropyridines (THPs). The remaining alkene group in the THP can be hydrogenated to provide isotopologues or stereoisotopomers of piperidines. There are currently 21 approved piperidine drugs with no substitution on the ring other than at the nitrogen. Placement of a deuterium at either C2 or C3 using a resolved tungsten complex or other dearomatization agent according to the presently disclosed method can make the piperidine ring chiral, thereby providing for study of the stereopharmacokinetics of these drugs.

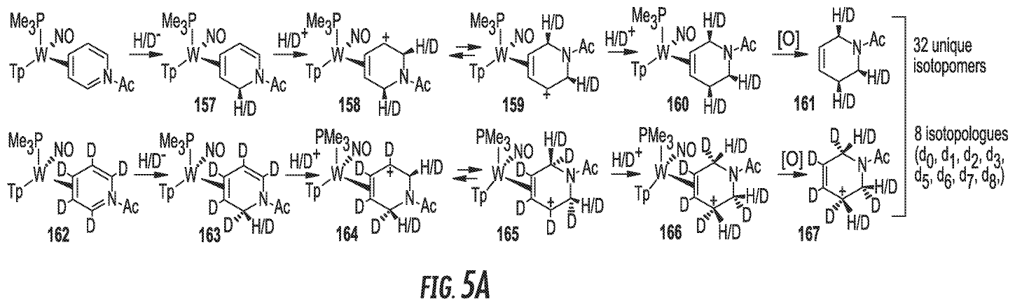

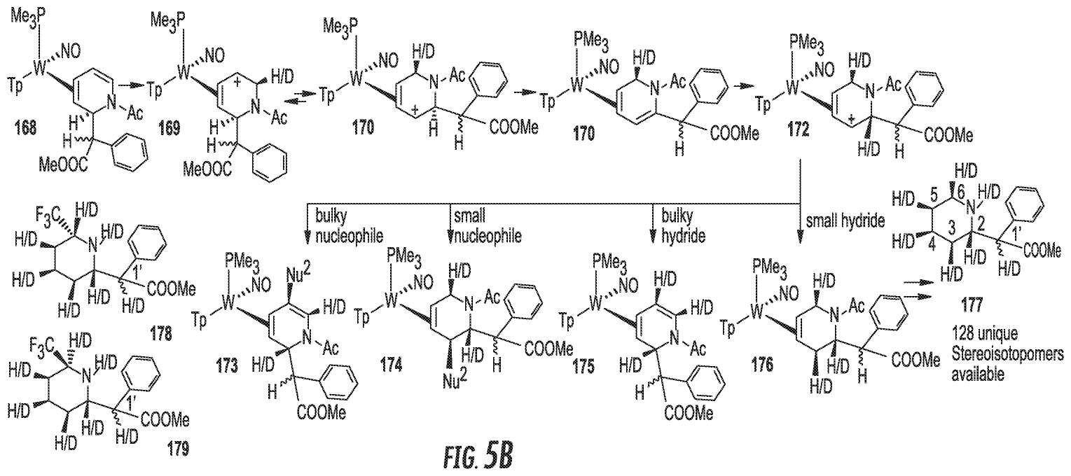

More particularly, each acid or hydride addition step of the presently disclosed method can be isolated, and, thus, each application of acid or hydride can be independently designated as H or D (or T), without isotopic scrambling. As shown in FIG. 5A, starting from a metal complex comprising a dihapto-coordinated N-acylated pyridinium ligand, prepared as previously described from pyridine borane,³⁵ or the corresponding metal complex prepared starting from pyridine-d₅ borane, the presently disclosed method can lead to 32 unique stereoisotopomers of unsubstituted THP (161, 167), in the form of 8 isotopologues, which can be prepared in isotopically and stereoisotopomerically pure form (given the availability of either enantiomer of the dearomatization agent). See FIG. 5A.

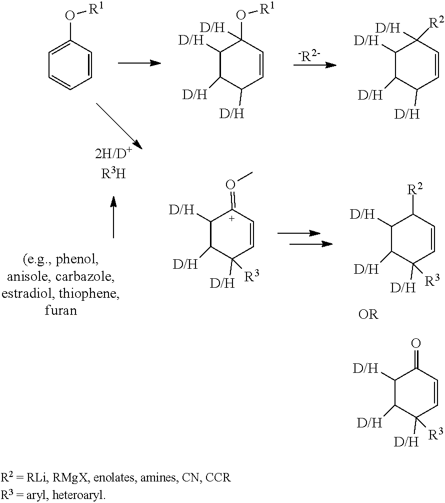

The presently disclosed method can also provide isotopologues or stereoisotopomers of substituted THPs, which can then be transformed into isotopologues or stereoisotopomers of substituted piperidines. Scheme 6, below, shows 2- and 3-substituted pyridines that can be used as the arene to prepare the first metal complex of the presently disclosed method. See Scheme 6, left. Possible products after decomplexation of a second metal complex provided from four stepwise additions (i.e., (1) H/D+, (2) nucleophile (H/D- or “R²”), (3) H/D+, and (4) nucleophile (H/D− or “R³”)) to a first metal complex prepared by complexation of pyridine, 2-substituted pyridine, or 3-substituted pyridine, followed by N-acylation or N-sulfonation (i.e., where R¹ is —C(═O)CH₃ (Ac), —C(═O)R, where R is alkyl; —S(═O)₂—Ar, or —S(═O)₂—NR₂) are shown in Scheme 6, right. Additional products can be prepared starting from pyridine-d₅. Nucleophiles R² and R³ can each be independently selected from dialkylzinc reagents, enolates (e.g., silyl enolates), primary or secondary amines (NR₂), cyanide salts, alkynides (CCR), and alkyl or aryl magnesium halides (RMgX or ArMgX). Pyridine substituents R⁴ and R⁵ can be alkyl, perfluoralkyl (e.g., CF₃), —CF₂R (where R is alkyl), phenyl (Ph), or pyridyl.

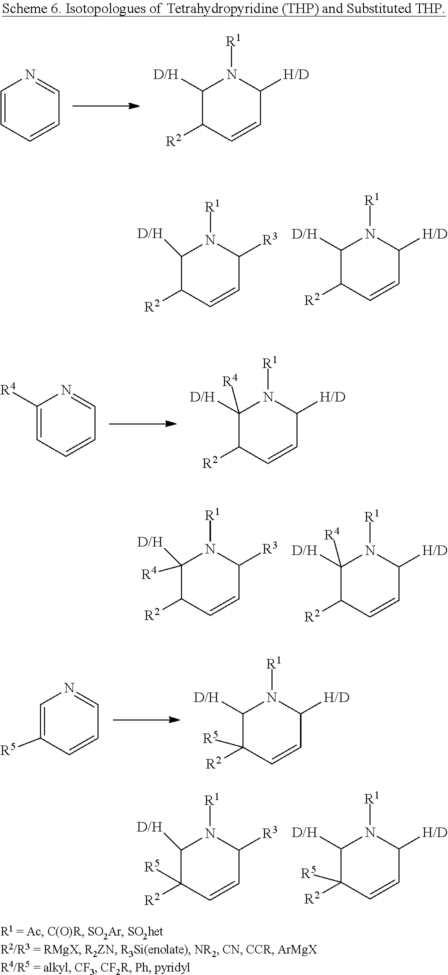

The ability to access substituted THPs is of note as it provides the ability to perform isotopic labeling for metabolite studies and pharmacokinetic and stereopharmacokinetic studies of drugs containing substituted piperidine moieties. There are currently 19 FDA approved drugs that have a piperidine group with only a C2 carbon substituent. One example is the psychostimulant methylphenidate (sold under various brand names such as Concerta and Ritalin). In particular, methylphenidate has been prescribed for decades for the treatment of Attention Deficit Hyperactivity Disorder (ADHD) and as a cognitive enhancer. The drug is known to block the pre-synaptic reuptake of dopamine and noradrenaline.

To date, fifteen different isotopologues of methylphenidate have been prepared involving C, O, and H isotopes. However, only one example involves deuteration of the piperidine ring, and it was exhaustive (d₉).^{32,33} Using the presently disclosed method outlined above, deuteriums (or tritiums) can be surgically installed at particular positions of the piperidine ring of methylphenidate starting from the metal complex 168, which can be prepared, for example, from the dihapto-coordinated N-acylated pyridinium complex shown in FIG. 5A via addition of an appropriate carbon nucleophile (e.g., an enolate of methyl 2-phenylethanoate) in a manner analogous to methods previously described.³⁵ See FIG. 5B. The resulting compounds can be of high isotopic purity and completely stereoselective. In addition to the ring carbons, the benzylic carbon (1′ in 177 in FIG. 5B), the nitrogen substituent, and the hydrogens added through hydrogenation of the THP alkene (C5 and C6 in 177 in FIG. 5B) can all be independently controlled. FIG. 5B also shows the intermediates formed when the final hydride addition is performed using a small hydride (e.g., NaBH₄) versus a bulky hydride (e.g., lithium borobicyclo[3.3.1]nonane (Li 9-BBN)) and the intermediates formed when the final addition is perfomed using a small nucleophile (e.g., a cyanine or acetylide salt) versus a bulky nucleophile (e.g., diethyl malonate or another substituted enolate). Alternatively, starting from trifluoropicoline, the 6-trifluoromethylated derivatives of methylphenidate can prepared as two different stereoisomers. See FIG. 5B, compounds 178, 179. This type of precision deuteration is not available through any conventional method. A multitude of derivatives of methylphenidate have been prepared. In all, 426 derivatives are listed in the Reaxys database that maintain the methylphenidate core. Of these, only one derivative (the 3-methoxy) has been prepared in which the piperidine ring has a substituent.³⁴

Accordingly, in some embodiments, the dihapto-coordinated ligand of the first metal complex is an N-acylated pyridinium salt, a N-tosylated pyridinium salt, an N-acylated substituted pyridinium salt or an N-tosylated pyridinium salt, and the method provides an isotopologue or a stereoisotopomer of a tetrahydropyridine (THP). In some embodiments, the method further comprises contacting the isotopologue or stereoisotopomer of the THP with a reducant (e.g., hydrogenation reagents, such as H₂ gas and a Pd catalyst) or an oxidant (e.g., mCPBA, MNDO, or OsO₄), thereby providing an isotopologue or a stereoisotopologue of a piperidine. For example, in some embodiments, the piperidine is methylphenidate.

### III. Isotopologues and Stereoisotopomers

In some embodiments, the presently disclosed subject matter provides an isotopologue or stereoisotopomer prepared according to the presently disclosed method or an isotopologue or stereoisotopomer prepared therefrom. In some embodiments, the isotopologue or stereoisotopomer has a structure of one of the isotopologues shown in Schemes 1-6, above. In some embodiments, the presently disclosed subject matter can provide an isotopologue or stereoisotopomer of a cyclohexane or a piperidine, which can be prepared by hydrogenation of an isotopologue or stereoisotopomer of cyclohexene or THP. In some embodiments, the isotopologue or stereoisotopomer is an epoxide formed by epoxidation of the alkene in a cyclohexene isotopologue or stereoisotopomer of the presently disclosed subject matter. In some embodiments, the isotopologue or stereoisotopomer prepared according to the presently disclosed method is a compound not previously prepared by another method or that is prepared in greater enantiomeric excess than by another method. For example, the isotopologue or stereoisotopomer can be a compound other than cyclohex-1-ene-1,2-d₂, cyclohex-1-ene-1-d, (R)-cyclohex-1-ene-3-d, or (3R,4R,5S,6S)-cyclohex-1-ene-3,4,5,6-d₄. In some embodiments, the isotopologue or stereoisotopomer can have an isotopic purity of greater than about 75% (e.g., greater than about 75%, 80%, 85%, 90%, 95%, 96%, 97%, 98%, or 99%). In some embodiments, the stereoisotopomer has an enantiomeric excess of greater than about 80%.

In some embodiments, the presently disclosed subject matter provides an isotopologue or stereoisotopomer of a cyclohexene or a substituted cyclohexene. In some embodiments, said isotopologue or stereoisotopomer comprises at least one hydrogen and at least one deuterium or tritium, (e.g., at least one cyclohexene ring carbon (e.g., one sp³ cyclohexene ring carbon) substituted by hydrogen and at least one cyclohexene ring carbon (e.g., one sp³ cyclohexene ring carbon) substituted by D or T. In some embodiments, the isotopologue or stereoisotopomer is not a compound selected from cyclohex-1-ene-1,2-d₂, cyclohex-1-ene-1-d, (R)-cyclohex-1-ene-3-d, or (3R,4R,5S,6S)-cyclohex-1-ene-3,4,5,6-d₄. In some embodiments, the isotopologue or stereoisotopomer has an isotopic purity of at least 75% (e.g., about 75%, 80%, 85%, 90%, 95%, 96%, 97%, 98%, or 99%). In some embodiments, the isotopologue or stereoisotopomer is a stereoisotopomer having an enantiomeric excess of about 80% or more.

In some embodiments, the isotopologue or stereoisotopomer of a substituted cyclohexene comprises a substituent selected from cyano, alkyl (e.g., saturated straight chain or branched alkyl, optionally C₁-C₅ alkyl) perfluoalkyl (e.g., CF₃ or another C₁-C₅ perfluoroalkyl), substituted alkyl (e.g., ester-substituted alkyl, such as —CH(CO₂Me)₂ or —C(CH₃)₂(CO₂Me)), alkylamino, dialkylamino, —SO₂-aryl, SO₂-substituted aryl, —SO₂—NR₂ (wherein each R is alkyl, aralkyl, or aryl) and —SF₅. In some embodiments, the substituent is selected from —CF₃, —CH₃, and CN.

In some embodiments, the isotopologue or stereoisotopomer has a structure of one of Formulas (Ia), (Ia), (Ib), (IIa), (IIb), (IIIa), and (IIIb):

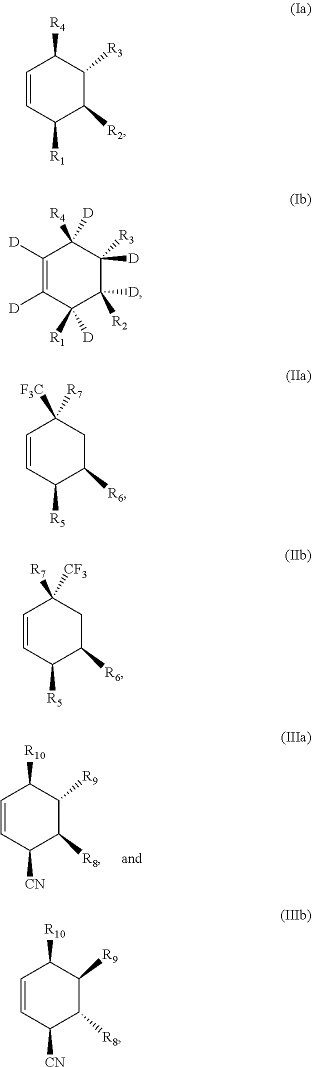

wherein: each of R₁, R₂, R₃, R₄, R₅, R₆, R₇, R₈, R₉, and R₁₀ is independently selected from H and D, where for Formula (Ia), at least one of R₁-R₄ is D; for Formula (Ib), at least one of R₁-R₄ is H; for Formulas (IIa) and (IIb) at least one of R₅-R₇ is D; and for Formula (IIIa) and (IIIb), at least one of R₈-R₁₀ is D.

For instance, in some embodiments, the isotopologue or stereoisotopomer has a structure of Formula (Ia) where one, two, three, or all four of R₁R₂, R₃, and R₄ is D. In some embodiments, one, two, or three of R₁, R₂, R₃, and R₄ is D. In some embodiments, the isotopologue or stereoisotopomer has a structure of Formula (Ib) where: R₁ and R₂ are D and R₃ and R₄ are H; R₁ is D and R₂, R₃, and R₄ are each H; or R₁-R₄ are each H. In some embodiments, the isotopologue or stereoisotopomer has a structure of Formula (IIa) wherein one or both of R₅ and R₆ is D and R₇ is H (e.g., where R₅ is D and R₆ and R₇ are each H; where R₆ is D and R₅ and R₇ are each H; or where R₅ and R₆ are each D and R₇ is H). In some embodiments, the isotopologue or stereoisotopomer has a structure of Formula (IIb) wherein R₅ and R₆ are each D and R₇ is H or D. In some embodiments, the isotopologue or stereoisotopomer has a structure of Formula (IIIa) wherein one of R₈-R₁₀ is D and the other two of R₈-R₁₀ are each H (e.g., where R₈ is D and R₉ and R₁₀ are each H). In some embodiments, R₈ and R₉ are each D and R₁₀ is H. In some embodiments, the isotopologue or stereoisotopomer has a structure of Formula (IIIb) where R₁₀ is H and one of R₈ and R₉ is D and one of R₈ and R₉ is H (i.e., where R₈ is D and R₉ is H or where R₈ is H and R₉ is D).

In some embodiments, the presently disclosed subject matter provides an isotopologue or stereoisotopomer of tetrahydropyridine or a substituted tetrahydropyridine, wherein the isotopologue or stereoisotopomer comprises one, two, three, four, five, six, or seven deuteriums attached to ring carbon atoms of the tetrahydropyridine. In some embodiments, the isotopologue or stereoisotopomer has a structure of one of Formulas (IVa) and (IVb):

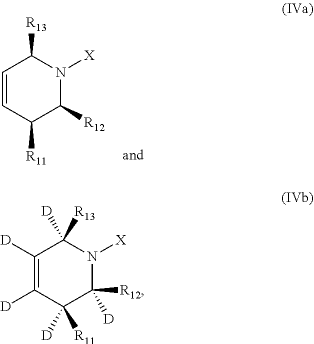

where X is H, D, acyl (e.g., —C(═O)R, where R is alkyl, such as Me), or sulfonyl (e.g., tosyl, —S(═O)₂—C₆H₅CH₃), and where each of R₁₁, R₁₂, and R₁₃ is independently selected from H and D, subject to the proviso that for Formula (IVa), at least one of R₁₁, R₁₂, and R₁₃ is D; and for Formula (IVb), at least one of R₁₁, R₁₂, and R₁₃ is H; or a salt thereof. In some embodiments, X is —C(═O)CH₃ or tosyl. In some embodiments, the isotopologue or stereoisotopomer has a structure of one of (Va) and (Vb):

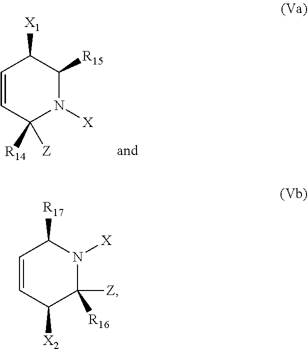

wherein X is H, D, acyl, or sulfonyl (e.g., tosyl); X₁ and X₂ are each selected from the group consisting of H, D, CN, alkyl, substituted alkyl, alkoxy, aryloxy, —NHR₂₄, —N(R₂₄)₂; and —P(R₂₄)₃; Z has a structure of the formula:

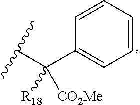

and
each of R₁₄, R₁₅, R₁₆, R₁₇, and R₁₈ is independently selected from H and D; and each R₂₄ is independently selected from alkyl, aralkyl, and aryl (e.g., C₁-C₅ alkyl); subject to the proviso that for Formula (Va) at least one of R₁₄, R₁₅, and Xi is D; and that for Formula (Vb) at least one of R₁₆, R₁₇, and X₂ is D; or a salt thereof. In some embodiments, X is —C(═O)CH₃ or tosyl. In some embodiments, the isotopologue or stereoisotopomer an isotopic purity of at least 75% (e.g., 80%, 85%, 90%, 95%, 96%, 97%, 98%, or 99%).

In some embodiments, the presently disclosed subject matter provides an isotopologue or a stereoisotopomer of methylphenidate or a 6-trifluoromethyl-substituted derivative thereof. In some embodiments, said isotopologue or stereoisotopomer has a structure of Formula (VI):

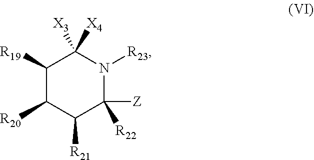

wherein X₃ and X₄ are each selected from H, D, and —CF₃; Z has a structure of the formula:

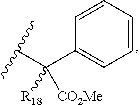

and
each of R₁₉, R₂₀, R₂₁, R₂₂ and R₂₃ is selected from H and D; or a salt thereof; and subject to the proviso that when one of X₃ and X₄ is —CF₃, the other of X₃ and X₄ is H or D; that when neither of X₃ and X₄ is —CF₃, X₃ is H and X₄ is H or D; and that at least one of R₂₁, R₂₂, and X₄ is D. In some embodiments, the isotopologue or stereoisotopomer has an isotopic purity of at least 75%.

In some embodiments, the presently disclosed subject matter provides an isotopologue or stereoisotopomer of a cyclohexadiene (e.g., prepared by decomplexation of a synthetic intermediate of a cyclohexene prepared according to the presently disclosed method) or of a dihydronaphthalene or a dihydrofuran, such as an isotopologue shown in the top two lines of Scheme 2.

### IV. Methods of Determining Absolute Configuration

Reliable and facile methods of determining the absolute configurations of stereoisotopomers, such as the stereoisotopomers of cyclohexenes, have been lacking in the literature. As described herein, dihapto-coordinated complexation of cyclohexene to a resolved tungsten metal complex (e.g., via ligand exchange of a cyclohexene with the benzene ligand of a resolved form of tungsten trispyrazolylborate nitrosomethyl trimethylphosphine benzene (e.g., (R)-WTp(NOMe)(PMe₃)(η²-benzene)) to provide a tungsten trispyrazolylborate nitrosomethyl trimethylphosphine dihapto-coordinated cyclohexene complex (e.g., (R)-WTp(NOMe)(PMe₃)(η²-cyclohexene) (see e.g., (R)-9 in FIG. 3F) provides full separation of the ¹H NMR signals in the ¹H NMR spectrum of the cyclohexene ligand. Thus, it is possible to use resolved forms of WTp(NOMe)(PMe₃)(η²-arene) complexes as reagents for the determination of the absolute configuration of stereoisotopomers of cyclohexene, i.e., by comparing the ¹H NMR spectrum of a complex of a stereoisotopomer of interest (e.g., a stereoisotopomer of cyclohexene having an unknown absolute configuration) prepared from said reagent to the ¹H NMR spectrum of of the corresponding tungsten complex comprising a dihapto-coordinated non-isotopically enriched cyclohexene. Proton signals that are suppressed in the spectrum of the complex of the stereoisotopomer of interest can indicate which protons are replaced by D or T.

Accordingly, in some embodiments, the presently disclosed subject matter provides a method of determining the absolute configuration of a stereoisotopomer of a cyclohexene (e.g., of a stereisotopomer of cyclohexene). In some embodiments, the method comprises: (a) contacting the stereoisotopomer of a cyclohexene with a tungsten metal complex, wherein said tungsten metal complex is a resolved form of WTp(NOMe)(PMe₃)(η²-benzene), wherein the contacting results in ligand exchange between the benzene and the stereoisotopomer of the cyclohexene, thereby providing a tungsten metal complex wherein the stereoisotopomer of the cyclohexene is dihapto-coordinated to tungsten (i.e., a WTp(NOMe)(PMe₃)(η²-cyclohexene) complex where the cyclohexene is known to comprise or is suspected of comprising one or more D or T); (b) collecting a proton nuclear magnetic resonance (NMR) spectrum of the tungsten metal complex comprising the dihapto-coordinated stereoisotopomer of the cyclohexene (i.e., the tungsten metal complex provided in step (a)); and (c) comparing the proton NMR spectrum collected in step (b) to a proton NMR spectrum of the corresponding tungsten metal complex wherein the dihapto-coordinated ligand is a non-isotopically enriched cyclohexene; thereby determining the absolute configuration of the stereoisotopomer (i.e., by determining which proton signals were suppressed in the spectrum collected in (b)). Suitable conditions for the ligand exchange step are described hereinabove in Section II. In some embodiments, the stereoisotopomer of the cyclohexene comprises at least one deuterium. In some embodiments, the cyclohexene comprises one or more substituent that is other than H, D, or T. In some embodiments the cyclohexene is an unsubstituted cyclohexene and does not contain any substituents other than H, D, or T.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Methods

NMR spectra were obtained on 500, 600 or 800 MHz spectrometers. Chemical shifts are referenced to tertramethylsilane (TMS) utilizing residual ¹H signals of the deuterated solvents as internal standards. Chemical shifts are reported in ppm and coupling constants (J) are reported in hertz (Hz). Infrared Spectra (IR) were recorded on a spectrometer as a glaze on a Horizontal Attenuated Total Reflectance (HATR) accessory, with peaks reported in cm⁻¹. Deuterium occupancy at stereospecific sites was determined from ¹H NMR data of a metal complex of a deuterated ligand, e.g., relying on the suppression of proton resonances that have been substituted for a deuterium. As confirmation of the stereochemical analysis, several free isotopomers were also analyzed by Molecular Rotation Resonance (MRR) spectroscopy. Deuterium integration was also assessed by high resolution mass spectrometry (HRMS).

Electrochemical experiments were performed under a nitrogen atmosphere. Most cyclic voltammetric data were recorded at ambient temperature at 100 mV/s, unless otherwise noted, with a standard three electrode cell from +1.8 V to −1.8 V with a platinum working electrode, acetonitrile (MeCN) solvent, and tetrabutylammonium (TBAH) electrolyte (˜1.0 M). All potentials are reported versus the normal hydrogen electrode (NHE) using cobaltocenium hexafluorophosphate (E_{1/2}=−0.78 V, −1.75 V) or ferrocene (E_{1/2}=0.55 V) as an internal standard. Peak separation of all reversible couples was less than 100 mV. All synthetic reactions were performed in a glovebox under a dry nitrogen atmosphere unless otherwise noted. All solvents were purged with nitrogen prior to use. Deuterated solvents were used as received from Cambridge Isotope Laboratories Inc. (Tewksbury, Mass., United States of America). When possible, pyrazole (Pz) protons of the (trispyrazolyl) borate (Tp) ligand were uniquely assigned (e.g., “Pz3B”) using two-dimensional NMR data. If unambiguous assignments were not possible, Tp protons were labeled as “Pz3/5 or Pz4”. All J values for Pz protons are 2 (±0.4) Hz.

### Example 1

### Synthesis of WTp(NO)(PMe₃)(η²-benzene-d₆) (17)

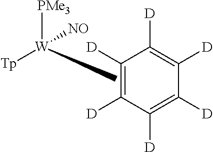

In an oven-dried 50 mL round bottom flask 25.0 g (297 mmol) of freshly degassed benzene-d₆ was added with a Teflon-coated magnetic stir bar. This solution was then stirred and 1 (0.404 g, 1.44 mmol) was added resulting in a homogeneous yellow reaction mixture which was allowed to stir for 18 hours. The reaction mixture was then added to 100 mL of pentane and the solvent volume was reduced by half in vacuo. Upon the evaporation of solvent a golden-yellow colored solid precipitated out of solution. This yellow solid was collected on a fine 15 mL fitted disc and washed with 2×15 mL of pentane to yield a yellow solid that was allowed to desiccate for 6 hours. A yellow solid was recovered (0.241 g, 59%). ¹H NMR features matched those reported previously for 1 with the absence of protons on the benzene ring.

### Example 2

### Synthesis of WTp(NO)(PMe₃)(η²-2,3-benzenium) (2) and WTp(NO)(PMe₃)(η²-1,2-benzenium) (3)

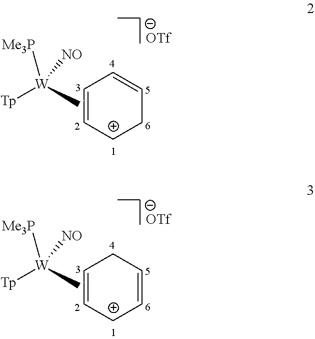

A 4-dram vial was charged with 5 mL of DCM and chilled to −30° C. for 15 minutes. 1 (1.52 g, 2.61 mmol) was then added, resulting in a heterogeneous yellow reaction mixture. Diphenylammonium triflate (DPhAT) (0.909 g, 2.84 mmol) was added to the reaction mixture at −30° C. resulting in the formation of a homogenous red solution. This solution was allowed to stand at −30° C. for 20 minutes. This reaction solution was then added to stirring Et₂O that had been chilled to −30° C. (60 mL) to yield a dull yellow precipitate. The yellow solid was collected on a 30 mL fine porosity fritted disc and washed with chilled Et₂O (2×20 mL). The isolated yellow solid was then desiccated for 2 hours yielding 2 and 3 (1.63 g, 2.24 mmol, 86% yield) in a 10:1 ratio.

Cyclic Voltammetry (CV) (MeCN at −6° C.) Ep,a=+0.70 V (NHE). IR: ν=2520 cm⁻¹, ν(NO)=1637 cm⁻¹. Complex 2 (major): ³¹P NMR (CD₃CN, δ): −7.44 (J_{WP}=274). ¹H NMR (CD₃CN, δ, 0° C.): 8.33 (1H, d, Pz3B/5B), 8.03 (1H, d, Pz3A), 8.02 (1H, d, Pz5C), 7.97 (1H, d, Pz3C), 7.94 (1H, d, Pz3B/5B), 7.80 (1H, d, Pz5A), 6.93 (1H, broad singlet, H1), 6.54 (1H, t, Pz4C), 6.49 (1H, t, Pz4B), 6.31 (1H, t, Pz4A) 6.17 (1H, m, H4), 5.04 (1H, m, H5), 4.91 (1H, t, J=7.2, H2), 4.24 (1H, m, H3), 4.16 (1H, d, J=28.3, H6-exo), 4.00 (1H, d, J=28.3, H6-endo), 1.18 ((H, d, J_{PH}=9.9, PMe₃). ¹³C NMR (CD₃CN, δ, 0° C.): 147.9 (Pz3A), 146.7 (Pz5C), 145.7 (C1), 143.0 (Pz3/5), 139.4 (Pz3/5), 139.3 (2C, Pz3/5), 126.1 (C4), 119.5 (C5), 109.2 (Pz4B), 108.7 (Pz4C), 107.8 (Pz4A), 95.1 (C2), 64.5 (C3), 30.7 (C6), 13.1 (PMe₃, d, J_{PC}=34.2). Complex 3 (minor): ¹H NMR (CD₃CN, δ, 0° C.): 8.31 (1H, d, Pz3/5), 8.11 (1H, d, Pz3/5), 8.01 (1H, d, Pz3/5), 7.92 (1H, d, Pz3C), 7.81 (1H, d, Pz3/5), 6.55 (1H, buried, H4), 6.52 (1H, t, Pz4), 6.49 (1H, buried, Pz4), 6.34 (2H, overlapping, Pz4 & H3), 5.89 (1H, m, H5), 4.71 (1H, t, J=7.5, H2), 4.65 (1H, m, H1), 4.34 (1H, d, J=25.8, H6-exo), 3.36 (d, J=25.8, 1H, H6-endo), 1.11 (9H, buried, PMe₃).

### Example 3

### Synthesis of WTp(NO)(PMe₃)(η²-1,2-1,3-cyclohexadiene) (4)

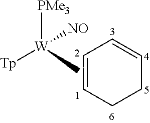

To a 4-dram vial was added 2 mL of MeCN and 1 (0.200 g, 0.344 mmol) to generate a heterogeneous yellow reaction mixture. This solution was then cooled to −30° C. DPhAT was added to this reaction mixture and the solution was allowed to stand at −30° C. Over 15 minutes a homogeneous red reaction mixture develops indicating the formation of 2 in solution. Separately a solution of 2 mL of MeOH was chilled to −30° C. and to this solution NaBH₄ (0.030 g, 0.79 mmol) was added. The NaBH₄/MeOH solution was then added to a dewar of liquid nitrogen and the solution was frozen. The homogenous red reaction mixture of 2 was then added to this frozen solution of MeOH and NaBH₄ and the reaction mixture was thawed at −30° C. After one hour the reaction mixture had turned to a homogenous orange color. A 60 mL coarse fritted porosity disc was filed with ˜5 cm of neutral alumina and set in Et₂O. The homogeneous orange solution was then filtered through the neutral alumina column and a light yellow band was eluted with 100 mL of Et₂O. The solvent was removed in vacuo until a pale yellow solid remained. This was re-dissolved in 2 mL of DCM and added to a 4-dram vial that contained 15 mL of sitting hexanes. This homogeneous yellow solution was subsequently allowed to cool at −30° C. for 16 hours. After being allowed to cool a crystalline product had developed on the sides of the vial. The organic layer was decanted and the product was then dried with N₂ (g) and allowed to desiccate for 8 hours before a mass was taken of the resultant light yellow crystalline product 4 (0.162 g, 81%). Characterization has been reported previously.

### Example 4

### Synthesis of WTp(NO)(PMe₃)(η²-1,2-1,3-cyclohexadiene) (5)

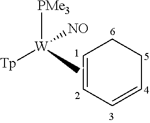

A mixture of complexes 4 and 5 (0.298 g, 0.511 mmol) were dissolved in 2 mL of DME and allowed to cool to −30 C over a period of 15 min. To this homogeneous light yellow solution was added HOTf (0.198 g, 1.311 mmol) and the reaction mixture turned to a homogeneous orange color. This reaction mixture was allowed to sit at −30° C. for 5 min and then subsequently added to 15 mL of standing ether at room temperature. A dark brown solid congregated on the bottom of the flask, and the organic layer was decanted before the solid was re-dissolved in 2 mL of DCM and again precipitated into 50 mL of stirring ether to generate a light yellow heterogeneous mixture. This solid was then dried under dynamic vacuum in a desiccator for 2 h and its identity as 6 was confirmed by NMR. This solid was then dissolved in DCM (2 mL) and allowed to sit at −30° C. In a separate 4-dram vial was added DBU (0.582 g, 3.82 mmol) along with DCM (1 mL) and this solution was also cooled to −30° C. over a course of 15 min. The DBU/DCM solution was then added to the DCM solution of 6 and upon addition a homogeneous pink reaction mixture develops. This reaction mixture was allowed to stand for 5 min at room temperature and then loaded onto a coarse 60 mL fritted disc that had been filled with ˜3 cm of basic alumina and set in ether. The pink band was eluted with 60 mL of ether and the solvent was then removed in vacuo. A light yellow film coats the bottom of the filter flask, this was re-dissolved in DCM (2 mL) and added to standing pentane (15 mL) and was allowed to sit at −30° C. for 16 hr. The next day a yellow crystalline material had formed at the sides of the vial. The organic layer was then decanted and the vial was dried under dynamic vacuum for 6 h to yield a fine yellow powder (0.102 g, 34%). Characterization has been reported previously.¹⁶

### Example 5

### Synthesis of WTp(NO)(PMe₃)(η²-1,2-cyclohexene) (7)

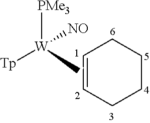

A 4-dram vial was charged with 1 mL of DME, complex 1 (0.200 g, 0.344 mmol) and cyclohexene (2.00 g, 24.3 mmol) and this heterogenous yellow reaction mixture was allowed to stir. After 27 hours the now homogenous purple reaction mixture was added to a 250 mL filter flask and the solvent was removed in vacuo. The resulting pink solid was redissolved in 2 mL of DCM and slowly added to 30 mL of stirring pentanes that had been chilled to −30° C. Upon addition a light pink solid precipitates out of solution. This light pink solid was collected on a 30 mL fine porosity fritted disc and subsequently washed with 3×5 mL of chilled pentane. The collected product was then desiccated for 2 hours to yield 7 as a fine off-white powder (0.083 g, 41.3%).

### Example 6

### Alternative Synthesis of WTp(NO)(PMe₃)(η²-1,2-cyclohexene) (7)

To a 4-dram vial 6 (0.107 g, 0.136 mmol) was dissolved in 1 mL of MeOH and chilled to −30° C. To this was added NaBH₄ that had been dissolved and chilled in a −30° C. MeOH solution. The solution of 6 was then added to the NaBH₄/MeOH solution and the resulting reaction mixture was allowed to sit at −30° C. for 16 hours. The reaction mixture was then eluted through a silica column on a fine 15 mL fritted disc and a light yellow band was eluted with ether. The solvent was then removed in vacuo and picked up in 1 mL of DCM. This was then added to 15 mL of sitting hexanes and allowed to sit at −30° C. for 2 hours during which a light yellow solid had precipitated from solution. This solid was then isolated on a fine 15 mL fritted disc, washed with 2×5 mL of hexanes to yield 7 as an off-white solid (0.058 g, 67%).

CV (MeCN) E_{p,a}=+0.28 V (NHE). IR: ν(BH)=2482 cm⁻¹, ν(NO)=1554 cm⁻¹. ¹H-NMR (CDCl₃, δ, 25° C.): 8.28 (1H, d, Pz3A), 8.08 (1H, d, Pz3B), 7.69 (1H, d, Pz5B), 7.64 (1H, d, Pz5C), 7.60 (1H, d, Pz5A), 7.24 (1H, d, Pz3C), 6.28 (1H, t, PzB), 6.20 (1H, t, Pz4A), 6.13 (1H, t, Pz4C), 3.00 (1H, m, H6exo) 2.92 (2H, overlap, m, H4-endo/exo), 2.71 (1H, m, H1), 2.64 (1H, m, H6-endo), 1.75 (2H, overlap, m, H6-endo/exo), 1.46 (2H, overlap, m, H5-endo/exo), 1.38 (1H, m, H2), 1.21 (9H, d, J_{PH}=8.1, PMe₃). ¹³C-NMR (CDCl₃, δ, 25° C.). 143.4 (Pz3B), 142.9 (Pz3A), 140.1 (Pz3C), 136.2 (Pz5), 135.6 (Pz5). 135.4 (Pz5), 106.3 (Pz4B), 105.6 (Pz4), 105.5 (Pz4), 53.8 (C2), 53.6 (C1, d, J_{PC}=10.6), 30.0 (C6, d, J_{PC}=4.2), 29.4 (C3), 24.9 (C4/5), 24.8 (C4/5), 14.0 (PMe₃, d, J_{PC}=27.5). ³¹P-NMR (CDCl₃, δ, 25° C.): −9.1 (J_{WP}=291, PMe₃). Anal. Calcd for 20C₁₈H₂₉BN₇OPWDCM: C, 36.79; H, 4.98; N, 16.76. Found: C, 37.35; H, 4.58; N, 16.69.

Characterization in MeCN-d₃. ¹H-NMR (MeCN-d₃, δ, 25° C.): 8.21 (1H, d, Pz3A), 8.03 (1H, d, Pz3B), 7.85 (1H, d, Pz5B), 7.78 (1H, d, Pz5C), 7.75 (1H, d, Pz5A), 7.35 (1H, d, Pz3C), 6.36 (1H, t, Pz4B), 6.27 (1H, t, Pz4A), 6.21 (1H, t, Pz4C), 3.04 (1H, m, H6exo) 2.93 (1H, m, H3-exo), 2.86 (1H, m, H3-endo), 2.72 (1H, m, H1), 2.63 (1H, m, H6-endo), 1.69 (1H, m, H4-endo), 1.66 (1H, m, H5-endo), 1.42 (2H, overlap, m, H4/H5-exo), 1.16 (1H, m, H2), 1.10 (9H, d, J_{PH}=8.2, PMe₃). ¹³C-NMR (MeCN-d₃, δ, 25° C.). 144.2 (Pz3B), 143.3 (Pz3A), 141.7 (Pz3C), 137.6 (Pz5), 136.9 (2C, overlapping Pz5), 107.4 (Pz4B), 106.9 (Pz4A), 106.6 (Pz4C), 53.5 (2C, overlapping C1 and C2), 30.7 (C6), 30.2 (C3), 25.6 (2C, overlapping C4/C5), 25.4 (2C, overlapping C4/C5), 13.8 (PMe₃, d, J_{PC}=27.5).

### Example 7

### Synthesis of WTp(NO)(PMe₃)(η²-1,4-cyclohexadiene) (8)

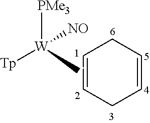

A 4-dram vial was charged with 1 mL of DME, complex 1 (0.301 g, 0.518 mmol) and 1,4-cyclohexadiene (2.00 g; 24.9 mmol) and this heterogenous yellow reaction mixture was allowed to stir. After 50 hours the homogenous purple reaction mixture was slowly added to 30 mL of stirring pentanes that had been chilled to −30° C. Upon addition, a light pink solid precipitates out of solution. This light pink solid was collected on a 30 mL fine porosity fritted disc and subsequently washed with 3×5 mL of chilled pentane. The collected product was then desiccated for 2 hours to yield 8 as a fine pink powder (0.191 g, 63%).

CV (MeCN) E_{p,a}=+0.27 V (NHE). IR: ν(BH)=2481 cm⁻¹, ν(NO)=1553 cm⁻¹. ¹H-NMR (acetone-d6, δ, 25° C.): 8.25 (1H, d, Pz3A), 8.08 (1H, d, Pz3B), 7.92 (1H, d, Pz5B), 7.88 (1H, d, Pz5C), 7.80 (1H, d, Pz5A), 7.49 (1H, d, Pz3C), 6.38 (1H, t, Pz4B), 6.28 (1H, t, Pz4A), 6.27 (1H, t, Pz4C), 5.83 (1H, m, H4) 5.78 (1H, m, H5), 3.55 (1H, m, H6-exo), 3.45 (2H, overlap, H3-exo and H3-endo), 3.10 (1H, d, J=17.8, H6-endo), 2.83 (1H, m, H1), 1.34 (1H, m, H2), 1.21 (9H, d, J_{PH}=8.2, PMe₃). 13C-NMR (acetone-d_{6, 6, 25}° C.). 144.0 (Pz3/5), 142.8 (Pz3/5), 141.5 (Pz3/5), 137.3 (Pz3/5), 136.7 (Pz3/5). 136.6 (Pz3/5), 127.7 (C4/5), 127.6 (C4/5), 107.1 (Pz4), 106.7 (Pz4), 106.2 (Pz4), 51.7 (C1, d, J_{PC}=11.9), 51.4 (C2), 30.1 (C3, buried under acetone), 29.2 (C6, buried under acetone, connected by HSQC), 13.6 (PMe₃, d, J_{PC}=27.4). ³¹P-NMR (CDCl3, δ, 25° C.): −8.9 (J_{WP}=292, PMe₃). Anal. Calcd for C₁₈H₂₇BN₇OPW: C, 37.08; H, 4.67; N, 16.82. Found: C, 37.35; H, 4.58; N, 16.69.

### Example 8

### Synthesis of WTP(NOMe)(PMe₃)(η²-cyclohexene)(OTf) (9)

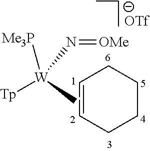

A 4-dram vial was charged with MeCN followed by 7 (0.203 g, 0.35 mmol). This heterogeneous yellow reaction mixture was cooled to −30° C. After 20 minutes methyl triflate (0.252 g, 1.54 mmol) that had been cooled to −30° C. was added and the reaction mixture was allowed to warm to room temperature over a period of 5 minutes. During this time the reaction mixture changed to a homogeneous green solution. After the reaction mixture turned to the homogeneous solution, 12 mL of Et₂O were added and the reaction mixture was allowed to sit at −30° C. for 30 minutes. The organic layer was decanted to reveal a crystalline yellow material sticking to the bottom of the vial. This was then washed with 2×5 mL of Et₂O and then dried under an N₂ (g) stream and subsequently desiccated over 4 hours, yielding 9 (0.116 g, 45%).

CV (MeCN) E_{p,a}=+1.2, E_{p,c}=−1.6 V (NHE). IR: ν(BH)=2511 cm⁻¹, ν(NO)=NA. ¹H-NMR (MeCN-d₃, δ, 25° C.): 8.51 (1H, d, Pz3A), 8.11 (1H, d, Pz3B), 8.02 (1H, d, Pz5B), 7.93 (2H, d, overlapping Pz5B/C), 7.47 (1H, d, Pz3C), 6.55 (1H, t, Pz4B), 6.48 (1H, t, Pz4A), 6.33 (1H, t, Pz4B), 4.03 (3H, s, NOMe), 3.97 (1H, m, H1), 3.57 (1H, m, H6-exo), 3.39 (1H, m, H3-exo), 3.14 (1H, m, H3-endo), 2.88 (1H, m, H6-endo), 2.34 (1H, m, H2), 1.65 (1H, m, H4-endo), 1.60 (1H, m H5-exo), 1.51 (1H, m, H5-endo), 1.48 (1H, m, H4-exo), 1.30 (9H, d, J_{PH}=9.4, PMe₃). ¹³C-NMR (MeCN-d₃, ν, 25° C.). 145.6 (Pz3B), 144.8 (Pz3A), 142.8 (Pz3C), 139.7 (Pz5A/C), 138.8 (Pz5A/C), 138.5 (Pz5B), 108.9 (Pz4B), 108.3 (Pz4C), 107.9 (Pz4A), 66.5 (NOMe), 64.5 (C1, J_{PC}=8.2), 63.2 (C2), 31.8 (C3), 31.4 (C6), 24.6 (C4), 23.9 (C5), 13.9 (PMe₃, d, J_{PC}=31.5). ³¹P-NMR (MeCN, δ, 25° C.): −10.76 (J_{WP}=268, PMe₃). Anal. Calcd for C₂₀H₃₂BN₇O₄F₃PSW: C, 32.06; H, 4.31; N, 13.09. Found: C, 32.00; H, 4.37; N, 12.92. ESI-MS: obsd (%), calcd (%): 598.1984 (83.66) 598.1985 (85.93), 599.2010 (76.17), 599.2011 (79.46), 600.2007 (100), 600.2009 (100), 601.2051 (37.70), 601.2052 (40.85), 602.2039 (83.65), 602.4041 (84.87).

### Example 9

### Synthesis of Diphenylammonium-d₂ Triflate (DPhAT-d₂)

Generation of an acidic deuterium source relied on stirring diphenyl ammonium triflate (DPhAT) (2.00 g, 6.25 mmol) in an excess of MeOD (50.0 g, 152 mmol). The mixture was allowed to stir for 16 hours and the solvent was removed in vacuo to yield d₂-DPhAT. Typical yields 90%.

### Example 10

### Incorporating Acidic Deuterium

Compound 1, 4, or 5 was dissolved in neat MeOD or a 20:1 solution of MeOD:HOTf was prepared. In cases of protonating 4, the reaction was done in neat MeOD (˜5 mL total volume) to mitigate contribution from proton impurity and the large KIE associated with the direct exo-protonation of the diene complex (k_{H}/k_{D}˜40). Acid sources were used in slight excess (1-1.1 equivalents) relative to the treated complex (1 or 4) at −30° C.

### Example 11

### Incorporatinq Deuteride

NaBD₄ was added to chilled MeOD and allowed to stand at reduced temperatures (−30° C. or at the freezing point of MeOD). Additionally NaBD₄ can also be solvated with 15-crown-5 ether in MeCN and chilled to −30° C. before use. In either case 2 or 6 could be added to this anionic deuterium source to yield an isotopologue of 4 or 7, respectively. Prepared deuteride sources were used in excess (2-10 molar equivalents) relative to the treated cationic complex.

### Example 12

### Representative Syntheses of Tungsten-Cyclohexene Isotopoloques

### Synthesis of 12

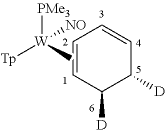

A solution of 1 (0.510 g, 0.878 mmol) in 2 mL of MeOD and 1 mL of MeCN-d₃ in a was prepared in a 4-dram vial and chilled to −30° C. Separately, a solution of HOTF (0.151 g, 1.00 mmol) in MeOD (5.01 g, 151 mmol) was prepared in a 4-dram vial as an acidic deuterium source and separately chilled to −30° C. over the course of 30 minutes. In a separate, oven-dried test tube MeOD (3 mL) was chilled to −60° C. in a chilled toluene bath. To this test tube was added NaBD₄ (0.205 g, 4.90 mmol) and the heterogeneous white reaction mixture was stirred over the course of 15 min. While this was stirring, the acidic solution of MeOH was added to the homogeneous yellow solution of 1. Upon addition, the reaction mixture turned to a homogeneous red color, indicative of the formation of 2 in solution. The solution of 2 was then allowed to sit for 15 min at −60° C. during which time it retained its red, homogeneous composition. After 15 min this reaction mixture was then added dropwise to the stirring test tube with NaBD₄ and MeOD, upon whose addition vigorously bubbling started to commence due to the evolution of H₂ gas. After all of 2 had been added to the test tube with the solution of NaBD₄ and MeOD, the reaction mixture was allowed to stir for 1.5 h before it was determined to be completed by ³¹P NMR. The reaction mixture was then removed from the −60° C. bath and the cap was loosened and the reaction mixture was allowed to warm to room temperature over the course of 15 min. During this time the reaction mixture turned from a light yellow to a light lime green color and vigorous bubbling started to occur. After the bubbling was judged to stop, the reaction mixture was eluted through a coarse 60 mL fritted disc filled with −3 cm of basic alumina that had been set in diethyl ether. A lime green band was then eluted with ether (100 mL) and the solvent was then removed in vacuo. Once a lime green solid film coats the bottom of the filter flask, the product was re-dissolved in a minimal amount of DCM (˜3 mL) and added to 15 mL of standing hexanes in a 4-dram vial. This was then allowed to sit at −30° C. over the course of 16 h, during which time a fine, lime green crystalline product has developed on the sides of the vial. The organic layer was then decanted and the product was dried with a N₂(g) stream and allowed to desiccate for six hours before a mass was collected of the lime green crystalline solid (0.365 g, 71%).

### Synthesis of 13

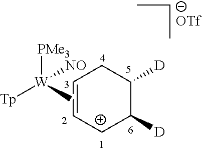

12 (0.365 g, 0.624 mmol) was dissolved in DME (3 mL) and cooled to −30° C. to generate a homogeneous yellow mixture in a 4-dram vial. Separately HOTf (0.215 g, 1.42 mmol) was dissolved in 2 mL of DME and also allowed to cool to −30° C. over a course of 15 min in a separate 4-dram vial. The acidic DME solution was then added dropwise to the standing solution of 3. This solution was allowed to stand at room temperature for 1 min and then was added dropwise to a solution of stirring ether (250 mL). Upon addition a fine, light yellow solid precipitated form solution. The solution was allowed to triturate for 10 min before the reaction mixture was filtered through a fine 15 mL porosity frit to yield a fine, yellow powder. This solid was then washed with ether (3×10 mL) and allowed to desiccate for six hours under dynamic vacuum before a mass was taken (0.365 g, 79%).

### Synthesis of 14

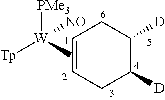

A 4-dram vial was charged with 13 (0.114 g, 0.155 mmol) and dissolved in MeOH (3 mL) to generate a homogeneous orange solution. This solution was chilled to −30° C. over a course of 15 min and to this solution was added NaBH₄ (0.040 g, 1.06 mmol). Upon addition to the solution some bubbling occurs. This solution was allowed to stand at −30° C. for one hour and turns from a homogeneous orange to a homogeneous yellow color. The solution was then allowed to stand at room temperature for 10 min before being diluted with ether (10 mL). Separately a 30 mL medium porosity frit was filled with ˜3 cm of silica and set in ether. The reaction mixture was then loaded onto this silica column and was filtered through by elution with ˜100 mL of ether total to elute a light yellow band. The solvent was then removed in vacuo and the product was re-dissolved in DCM (2 mL) and added to a 4-dram vial of standing pentane (15 mL). This solution was allowed to stand at −30° C. for 16 h before the solvent was again stripped to dryness to yield a fine off-white solid (0.061 g, 65%).

### Synthesis and Characterization of 47

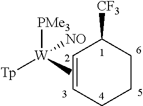

To a 4-dram vial were added WTp(NO)(PMe₃)(η²-1-(trifluoromethyl)cyclohexa-1,3-diene)²⁸ (50 mg, 0.077 mmol) followed by acetone (1.3 mL, −30° C.), resulting in a heterogeneous mixture. A 1M solution of HOTf in acetonitrile (0.1 mL, 0.1 mmol, −30° C.) was then added, resulting in a homogeneous yellow solution, which was allowed to stir for 19 h at room temperature. This orange solution was then cooled to −30° C. for 30 min. In a separate 4-dram vial, NaBH₄ (10 mg, 0.26 mmol) and MeOH (0.2 mL, −30° C.) were combined. The reaction solution was then added to the NaBH₄ mixture with stirring, and this solution was left at −30° C. for 1 h. The reaction solution was allowed to warm to room temperature for 30 min, before precipitation was induced by adding H₂O (3 mL). The resulting tan solid was collected on a 15 mL fine porosity fritted disc, washed with H₂O (2×5 mL), and pentane (2×3 mL, −30° C.) and desiccated, yielding 47 (40 mg, 0.061 mmol, 79% yield).

¹H NMR (acetone-d₆, δ): 8.24 (d, 1H, PzA3), 8.15 (d, 1H, PzB3), 7.96 (d, 1H, PzB5), 7.93 (d, 1H, PzC5), 7.81 (d, 1H, PzA5), 7.50 (d, 1H, PzC3), 6.42 (t, 1H, PzB4), 6.31 (t, 1H, PzC4), 6.28 (t, 1H, PzA4), 3.49 (m, 1H, H1), 3.17 (m, 1H, H4-endo), 2.98 (m, 1H, H4-exo), 2.55 (t, J=10.8, 1H, H2), 2.07 (buried, 1H, H6-exo), 1.76 (dm, J=14.1, 1H, H6-endo), 1.59 (m, 1H, H5-exo), 1.57 (m, 1H, H5-endo), 1.43 (m, 1H, H3), 1.18 (d, J=8.3, 9H, PMe₃). ¹³C NMR (acetone-d₆, δ): 144.2 (PzB3), 143.0 (PzA3), 141.7 (PzC3), 137.7 (Pz5), 137.0 (Pz5), 136.8 (Pz5), 133.2 (q, JCF=282.3, CF₃), 107.3 (PzB4), 106.9 (PzC4), 106.3 (PzA4), 52.0 (C3), 47.9 (d, J_{PC}=13.0, C2), 43.9 (m, C1), 29.1 (C4), 22.6 (C6), 20.9 (C5), 13.0 (d, J_{PC}=27.8, PMe₃). ESI-MS: obsd (%), calcd (%): 652.1702 (84.59), 652.1702 (85.97), 653.1727 (76.24), 653.1728 (79.46), 654.1724 (100), 654.1726 (100), 655.1770 (36.53), 655.1769 (40.83), 656.1757 (80.76), 656.1758 (84.86).

### Characterization for 48

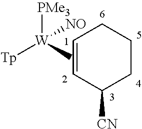

CV (MeCN) E_{p,a}=0.61 V (NHE). IR: (BH)=2513 cm⁻¹, ν(CN)=2228 cm⁻¹, ν(NO)=1558 cm⁻¹. ¹H-NMR (CDCl₃, δ, 25° C.): 8.05 (2H, overlapping d, Pz3A and Pz3B), 7.71 (1H, d, Pz5A), 7.69 (1H, d, Pz5C), 7.64 (1H, d, Pz5B), 7.24 (1H, d, Pz3C), 6.31 (1H, t, Pz4A), 6.26 (1H, t, Pz4B), 6.19 (1H, t, Pz4C), 3.99 (1H, broad s, H3), 3.12 (1H, m, H6-endo), 2.72 (1H, m, H6-exo), 2.68 (1H, m, H1), 2.07 (1H, m, H4-endo), 1.77 (1H, m, H4-endo), 1.70 (2H, overlapping, H5-exo and H5-endo), 1.38 (1H, d, J=11.1, H2), 1.18 (9H, d, J_{PH}=8.3, PMe₃). ³¹P-NMR (CDCl₃, δ, 25° C.): −10.55 (J_{WP}=285)¹³C-NMR (CDCl₃, δ, 25° C.): 143.3 (Tp3/5), 142.4 (Tp3/5), 140.1 (Tp3/5), 136.6 (Tp3/5), 136.1 (Tp3/5), 136.1 (Tp3/5), 128.1 (CN), 106.6 (Tp4), 106.1 (Tp4), 106.0 (Tp4), 52.3 (C2), 49.7 (C1, J_{PC}=11.6), 31.0 (C3), 28.7 (C6, d, J_{PC}=3.5), 27.5 (C4), 22.0 (C5), 13.8 (PMe₃, J_{PC}=27.9).

Synthesis was analogous to 48-4-exo, 5-endo-d₂ (vida infra) but utilized only the proteated versions of the sodium borohydride and methanol.

### Synthesis of 52

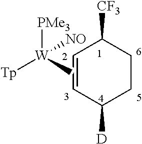

To a 4-dram vial were added WTp(NO)(PMe₃)(η²-1-(trifluoromethyl)cyclohexa-1,3-diene)²⁸ (50 mg, 0.077 mmol) followed by acetone (1.3 mL, −30° C.), resulting in a heterogeneous mixture. A 1M solution of HOTf in acetonitrile (0.1 mL, 0.1 mmol, −30° C.) was then added, resulting in a homogeneous yellow solution, which was allowed to stir for 19 h at room temperature. This orange solution was then cooled to −30° C. for 30 min. In a separate 4-dram vial, NaBD₄ (11 mg, 0.26 mmol) and MeOD (0.2 mL, −30° C.) were combined. The reaction solution was then added to the NaBD₄ mixture with stirring, and this solution was left at −30° C. for 1 h. The reaction solution was allowed to warm to room temperature for 30 min, before precipitation was induced by adding H₂O (3 mL). The resulting tan solid was collected on a 15 mL fine porosity fritted disc, washed with H₂O (2×5 mL), and pentane (2×3 mL, −30° C.) and desiccated, yielding 52 (48 mg, 0.073 mmol, 95% yield). 94% deuterium incorporation of C4. Deuterium incorporation determined by integration of ¹H NMR signal at 2.98 ppm.

### Synthesis of WTp(NO)(PMe₃)(η²-1-trifluoromethyl-5-deutero-1,3-cyclohexadiene)

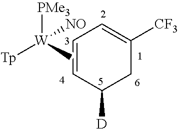

To a 4-dram vial charged with a stir pea were added WTp(NO)(PMe₃)(η²-2,3-α,α,α-trifluorotoluene) (200 mg, 0.308 mmol) followed by MeCN (1.8 mL, −30° C.), resulting in a heterogeneous mixture. A 1M solution of HOTf in MeCN (0.32 mL, 0.32 mmol, −30° C.) was immediately added with stirring, resulting in a homogeneous orange solution, which was allowed to sit for 20 min at −30° C. In a separate 4-dram vial, NaBD₄ (39 mg, 0.93 mmol) was dissolved in MeOH (1 mL, −30° C.). This chilled hydride solution was then added to the orange reaction solution dropwise with vigorous stirring. The reaction was allowed to sit at −30° C. for 10 h, at which point a solid had precipitated out of solution. The reaction solution was stirred at room temperature for 5 min, before further precipitation was induced by adding H₂O (5 mL). The resulting solid was collected on a 15 mL fine porosity fritted disc, washed with H₂O (2×5 mL), and pentane (2×4 mL, −30° C.) and desiccated (179 mg, 0.275 mmol, 89% yield). Greater than 95% deuterium incorporation at C5.

### Synthesis of 53

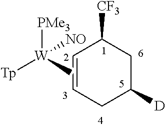

NaBH₄ (14 mg, 0.37 mmol) was dissolved in d₄-methanol (0.4 mL, −30° C.) in a 4-dram vial charged with a stir pea. WTp(NO)(PMe₃)(η²-1-(trifluoromethyl)cyclohexa-1,3-diene-5-d) (50 mg, 0.077 mmol) was added to another 4-dram vial charged with a stir pea. In a separate 4-dram vial, CD₃CN (0.8 mL, −30° C.) and a 1M solution of HOTf in MeCN (0.16 mL, 0.16 mmol, −30° C.) were combined and cooled to −40° C. for 20 min. The acid solution was added to the vial with tungsten complex, with stirring, resulting in a homogeneous yellow solution, which was allowed to sit for 2 min at −40° C. The yellow reaction solution was then added to the chilled hydride mixture with stirring. The pale yellow reaction was left at −40° C. for 8 min then moved to the freezer at −30° C. for 40 min, during which time solid precipitated out of solution. The heterogeneous reaction mixture was removed from the freezer and further precipitation was induced by adding H₂O (2 mL). The pale yellow solid was collected on a 15 mL fine porosity fritted disc, washed with H₂O (5 mL), and pentane (2×3 mL, −30° C.) and desiccated, yielding 53 (35 mg, 0.054 mmol, 70% yield). 95% deuterium incorporation at C5. Deuterium incorporation determined by integration of ¹H NMR signal at 1.60 ppm.

### Synthesis of 54

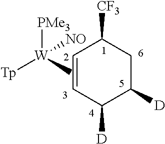

NaBD₄ (14 mg, 0.33 mmol) was dissolved in d₄-methanol (0.4 mL, −30° C.) in a 4-dram vial charged with a stir pea. WTp(NO)(PMe₃)(η²-1-(trifluoromethyl)cyclohexa-1,3-diene-5-d) (50 mg, 0.077 mmol) was added to another 4-dram vial charged with a stir pea. In a separate 4-dram vial, CD₃CN (0.8 mL, −30° C.) and a 1M solution of HOTf in MeCN (0.16 mL, 0.16 mmol, −30° C.) were combined and cooled to −40° C. The acid solution was added to the vial with tungsten complex, with stirring, resulting in a homogeneous yellow solution, which was allowed to sit for 2 min at −40° C. The yellow reaction solution was then added to the chilled hydride mixture with stirring. The pale yellow reaction was left at −40° C. for 8 min then moved to the freezer at −30° C. for 40 min, during which time solid precipitated out of solution. The heterogeneous reaction mixture was removed from the freezer and further precipitation was induced by adding H₂O (2 mL). The pale yellow solid was collected on a 15 mL fine porosity fritted disc, washed with H₂O (5 mL), and pentane (2×3 mL, −30° C.) and desiccated, yielding 53 (36 mg, 0.055 mmol, 71% yield). 95% deuterium incorporation at C4 and 95% deuterium incorporation at C5. Deuterium incorporation determined by integration of ¹H NMR signals at 2.98 and 1.60 ppm.

### Synthesis of 55

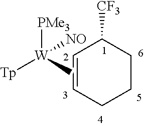

To a 4-dram vial charged with a stir pea were added WTp(NO)(PMe₃)(η²-2,3-α,α, α-trifluorotoluene) (100 mg, 0.154 mmol) followed by MeCN (0.45 mL, −30° C.), resulting in a heterogeneous mixture. A 1M solution of HOTf in MeCN (0.3 mL, 0.3 mmol, −30° C.) was immediately added with stirring, resulting in a homogeneous orange solution, which was allowed to sit for 20 min at −30° C. In a separate 4-dram vial, NaCNBH₃ (29 mg, 0.46 mmol) was dissolved in MeOH (0.45 mL, −30° C.). This chilled hydride solution was then added to the orange reaction solution dropwise with vigorous stirring. After addition of the hydride solution a solid precipitated out of the reaction mixture. The reaction was allowed to sit at −30° C. for 1.5 h, then the pale yellow solid was collected on a 15 mL fine porosity fritted disc, washed with H₂O (2×5 mL), and pentane (2×5 mL, −30° C.) and desiccated, yielding 55 (81 mg, 0.12 mmol, 78% yield). E_{p,a}=+0.49 V (NHE). IR: ν(BH)=2487 cm⁻¹, ν(NO)=1548 cm⁻¹. Anal. Calc'd for C₁₉H₂₆BF₃N₇OPW.2CH₂Cl₂: C, 30.65; H, 3.92; N, 11.91. Found: C, 31.08; H, 4.10; N, 12.21. ³¹P NMR (acetone-d₆, δ): −13.84 (J_{WP}=272). ¹H NMR (acetone-d₆, δ): 8.18 (d, 1H, PzB3), 7.97 (d, 1H, PzB5), 7.96 (d, 1H, PzC5), 7.80 (d, 1H, PzA5), 7.73 (d, 1H, PzA3), 7.40 (d, 1H, PzC3), 6.43 (t, 1H, PzB4), 6.33 (t, 1H, PzC4), 6.28 (t, 1H, PzA4), 4.01 (m, 1H, H1), 3.18 (m, 1H, H4-exo), 2.96 (t, J=12.1, 1H, H2), 2.17 (d of m, J=14.4, 1H, H4-endo), 1.93 (m, 1H, H6-endo), 1.81 (m, 1H, H5-endo), 1.63 (m, 1H, H6-exo), 1.57 (m, 1H, H5-exo), 1.29 (d, J=12.1, 1H, H3), 1.04 (d, J_{PH}=8.3, 9H, PMe₃). ¹³C NMR (acetone-d₆, δ): 144.0 (PzA3), 143.1 (PzB3), 141.1 (PzC3), 137.5 (PzC5), 137.2 (PzB5), 136.7 (PzA5), 131.1 (q, J_{CF}=278.4, CF₃), 107.5 (PzB4), 107.1 (PzC4), 106.6 (PzA4), 58.1 (C3), 46.7 (d, J=14.6, C2), 45.0 (q, J_{CF}=24.7, C1), 25.3 (C4), 20.4 (C6), 19.8 (C5), 13.3 (d, J_{PC}=29.1, PMe₃).

### Synthesis of 56

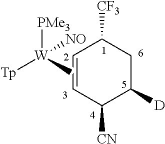

To a 4-dram vial charged with a stir pea were added WTp(NO)(PMe₃)(η²-2,3-α,α, α-trifluorotoluene) (100 mg, 0.154 mmol) followed by CD₃CN (0.35 mL, −30° C.), resulting in a heterogeneous mixture. A 1M solution of HOTf in MeCN (0.39 mL, 0.39 mmol, −30° C.) was immediately added with stirring, resulting in a homogeneous orange solution, which was allowed to sit for 20 min at −30° C. In a separate 4-dram vial, NaCNBD₃ (30 mg, 0.46 mmol) was dissolved in 1:1 methanol:d₄-methanol (0.8 mL, −30° C.). This chilled hydride solution was then added to the orange reaction solution dropwise with vigorous stirring. The reaction was allowed to sit at −30° C. for 3 h, at which point a solid had precipitated out of solution. The pale yellow solid was collected on a 15 mL fine porosity fritted disc, washed with H₂O (5 mL), and pentane (2×5 mL, −30° C.) and desiccated, yielding 56 (75 mg, 0.11 mmol, 71% yield). 10% deuterium incorporation at C1, 81% deuterium incorporation at C5, and 60% deuterium incorporation at C4. Deuterium incorporation determined by integration of ¹H NMR signals at 4.01, 3.18, and 1.57 ppm.

### Synthesis of 57

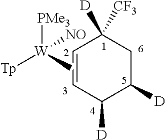

To a 4-dram vial charged with a stir pea were added WTp(NO)(PMe₃)(η²-2,3-α,α, α trifluorotoluene) (100 mg, 0.154 mmol) followed by CD₃CN (0.35 mL, −30° C.), resulting in a heterogeneous mixture. A 1M solution of HOTf in MeCN (0.39 mL, 0.39 mmol, −30° C.) was immediately added with stirring, resulting in a homogeneous orange solution, which was allowed to sit for 20 min at −30° C. In a separate 4-dram vial, NaCNBD₃ (30 mg, 0.46 mmol) was dissolved in d₄-methanol (0.8 mL, −30° C.). This chilled hydride solution was then added to the orange reaction solution dropwise with vigorous stirring. The reaction was allowed to sit at −30° C. for 4 h, at which point a solid had precipitated out of solution. The pale yellow solid was collected on a 15 mL fine porosity fritted disc, washed with H₂O (2×5 mL), and pentane (2×5 mL, −30° C.) and desiccated, yielding 57 (75 mg, 0.11 mmol, 71% yield). 77% deuterium incorporation at C1, 84% deuterium incorporation at C5, and 93% deuterium incorporation at C4. Deuterium incorporation determined by integration of ¹H NMR signals at 4.01, 3.18, and 1.57 ppm.

### Synthesis of 58

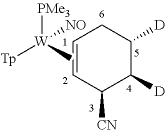

¹H NMR Characterization of 58-4-exo, 5-endo-d₂ matches that of 48 but with an 90% loss of ¹H signal intensity at 2.07 ppm and a 93% loss of ¹H signal intensity at 1.77 (isotopic purity estimated by ¹H NMR). There is an unintended 15% underintegration at 3.12 representing scrambling of deuterium in the H6-exo position.

A test tube was charged with NaCN (0.235 g, 4.80 mmol) and MeOD (2 mL) along with a small stir bar and was allowed to stir at room temperature for 2 h to dissolve. This reaction mixture was then transferred to a −60° C. toluene bath and to this solution was added HOTf (3 drops). Separately a solution of 13 (0.200 g, 0.272 mmol) was dissolved in a solution of MeOD (2 mL) and propionitrile (1 mL). This homogeneous yellow reaction mixture was then transferred to the a −60° C. toluene bath. After sitting in the reduced temperature toluene bath for 30 min the solution of 13 was added dropwise to the stirring solution of NaCN/MeOD at −60° and this light yellow solution was allowed to stir at −60°. After 2 h of stirring the reaction was determined to be completed by a ³¹P NMR experiment and the reaction vessel was removed from the box and diluted with 50 mL of DCM. The reaction mixture was then added to a 100 mL solution of DI H₂O saturated with NaCl (brine solution) in a 250 mL separatory funnel and the mixture was extracted with 3×50 mL DCM. The organic layers were then collected and dried over anhydrous MgSO₄ for 15 min. The DCM mixture was then eluted through a medium 60 mL medium porosity frit and the MgSO₄ on the frit was washed with DCM (50 mL total) to dissolve any remaining product. The DCM was then removed under reduced pressure until a light brown film remained. The film was re-dissolved in 3 mL of DCM and added to 100 mL of stirring pentane. Upon addition a light pink solid precipitated from solution. This heterogeneous solution was allowed to triturate for 10 min before the reaction mixture was filtered through a fine 15 mL frit. The isolate light pink solid was then washed with 2×10 mL pentane and allowed to desiccate under dynamic vacuum for 3 h. A mass was taken of the fine light pink solid (0.095 g, 57%).

### Synthesis of 59

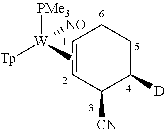

¹H NMR Characterization of 59-4-exo-di matches that of 48 but with an 87% loss of ¹H signal intensity at 1.77 ppm (isotopic purity estimated by ¹H NMR).

To a 4-dram vial was added 2 mL of propionitrile and 1 (0.252 g, 0.434 mmol) to generate a heterogeneous yellow reaction mixture. This solution was then cooled to −30° C. DPhAT was added to this reaction mixture and the solution was allowed to stand at −30° C. Over 15 min a homogeneous red reaction mixture develops indicating the formation of 2 in solution. Separately a solution of 2 mL of MeOH was chilled to −60° C. in a toluene bath and to this solution NaBD₄ (0.078 g, 1.86 mmol) was added. The NaBD₄/MeOH solution was then allowed to stir in a −60° C. The homogenous red reaction mixture of 2 was then added to this −60° C. cooled solution of MeOH and NaBD₄. After seven hours the reaction mixture had turned to a homogenous orange color and was removed from the −60° C. toluene bath and diluted with 40 mL of Et₂O and allowed to stir for 10 min at room temperature. A 60 mL medium fritted porosity disc was filled with ˜5 cm of silica and set in Et₂O. The homogeneous orange solution was then filtered through the silica column and a light yellow band was eluted with ˜100 mL of Et₂O. The solvent was removed in vacuo until a pale yellow solid remained. This was re-dissolved in 2 mL of DCM and added to a 4-dram vial that contained 15 mL of sitting hexanes. This homogeneous yellow solution was subsequently allowed to cool at −30° C. for 16 h. After being allowed to cool a light green crystalline product had developed on the sides of the vial. The organic layer was decanted and the product was then dried with N₂ (g) and allowed to desiccate for 16 h under static vacuum before its identity was confirmed by ¹H NMR. The resulting lime green solid was subsequently dissolved in 2 mL of MeOH and cooled to −30° C. Separately, a 4-dram vial was charged with NaCN (0.250 g, 5.1 mmol) along with MeOH and allowed to stir for 15 min at room temperature. This reaction mixture was then transferred to a −60° C. toluene bath and allowed to stir. To the −30° C. MeOH solution of 4-6,exo-d1 added HOTf (0.102 g, 0.680 mmol). Upon addition the reaction mixture goes from a heterogeneous green to a homogeneous yellow solution. This solution of 4-6,exo-d₁ was then cooled in the −60° C. toluene bath over a course of 10 mintues before it was added dropwise to the stirring solution of NaCN/MeOH that had been stirring in the −60° C. toluene bath. This solution was allowed to stir for 5 h at −60° C. before it was removed from the toluene bath. The reaction mixture was diluted with ˜50 mL of Et₂O and loaded onto a 30 mL medium fritted porosity disc that was filled with ˜5 cm of silica and set in Et₂O. A lime green band was collected upon elution with 100 mL of Et₂O and the solvent was removed in vacuo to reveal a lime green solid. This solid was then dissolved in 2 mL of DCM and added to 15 mL of standing pentane in a 4-dram vial and allowed to sit at −30° C. for 16 hr during which time a white solid precipitated out of solution. The next day the white solid was allowed to triturate in a minimal amount of MeCN given that the desired product was marginally soluble in MeCN. The organic layer was then decanted and the reaction mixture was dried with N₂(g) and allowed to desicatte for 3 h under active vacuum before a mass was taken (0.048 g, 18% overall yield).

### Synthesis of 60

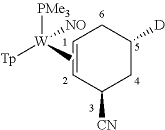

¹H NMR Characterization of 48-5-endo-di matches that of 48 but with an 85% loss of ¹H signal intensity at 1.70 ppm (isotopic purity estimated by ¹H NMR). Although overlap is present with the H5-endo proton, the precursor compound shows suppression of the expected endo-proton.

To a 4-dram vial added 1 (0.264 g, 0.454 mmol) along with propionitrile (2 mL) and this was allowed to sit at −30° C. Separately a solution of NaBH₄ (0.111 g, 2.92 mmol) was added to a −60° C. solution of MeOD (2 mL) in a test tube. In another 4-dram vial diphenylammonium triflate (DPhAT, 0.175 g, 0.546 mmol) was dissolved in MeOD (2.78 g, 83.9 mmol) and allowed to sit at −30° C. for 10 min before it was added to the solution of 1 in proptionitrile. This reaction mixture was then transferred to a −60° C. toluene bath in a 4-dram vial and allowed to cool over a period of 10 min. This reaction mixture, now a homogeneous red coloration, was then added to the −60° C. cooled solution of stirring NaBH₄/MeOD. Upon addition some bubbling occurs and this reaction mixture was allowed to stir for 5 h at −60° C. After 5 h the reaction mixture was removed from the −60° C. toluene bath and diluted with 40 mL of Et₂O and allowed to stir for 10 min at room temperature. A 60 mL medium fritted porosity disc was filled with ˜5 cm of silica and set in Et₂O. The homogeneous orange solution was then filtered through the silica column and a light yellow band was eluted with ˜100 mL of Et₂O. The solvent was removed in vacuo until a pale yellow solid remained. This was re-dissolved in 2 mL of DCM and added to a 4-dram vial that contained 15 mL of sitting hexanes. This homogeneous yellow solution was subsequently allowed to cool at −30° C. for 16 h. After being allowed to cool a light green crystalline product had developed on the sides of the vial. The organic layer was decanted and the product was then dried with N₂ (g) and allowed to desiccate for 16 h under static vacuum before its identity was confirmed by ¹H NMR as 2-5-endo-d1. This solid was then combined with MeOH (2 mL) in a 4-dram vial and allowed to cool to −30° C. over a course of 15 min. Separately a large test tube vial was charged with MeOH (2 mL), NaCN (0.240 g, 4.90 mmol) and this heterogeneous solution was allowed to stir at room temperature for 15 min. This solution was then transferred to a −60° C. toluene bath and allowed to stir for 10 min. To the −30° C. MeOH solution of 2-5-endo-d1 added HOTf (0.102 g, 0.680 mmol) that had also been cooled to −30° C. This solution, a light homogeneous yellow solution was transferred to the −60° C. toluene bath and allowed to stir for 5 min. This homogeneous yellow solution was then added dropwise to the −60° C. solution of NaCN/MeOH and allowed to stir for 5 h. This solution was allowed to stir for 5 h at −60° C. before it was removed from the toluene bath. The reaction mixture was diluted with ˜50 mL of Et₂O and loaded onto a 30 mL medium fritted porosity disc that was filled with ˜3 cm of silica and set in Et₂O. A lime green band was collected upon elution with 100 mL of Et₂O and the solvent was removed in vacuo to reveal a lime green solid. The next day the white solid was allowed to triturate in a minimal amount of MeCN given that the desired product was marginally soluble in MeCN. The organic layer was then decanted and the reaction mixture was dried with N₂ (g) and allowed to desicatte for 3 h under active vacuum before a mass was taken (0.048 g, 17% overall yield).

### Synthesis of 61

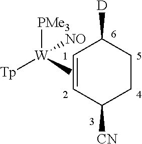

¹H NMR Characterization of 61-6-exo-d, matches that of 48 but with an 99% loss of ¹H signal intensity at 3.12 ppm (isotopic purity estimated by ¹H NMR, in this case proton impurity is beyond ¹H NMR detection limit).

WTp(NO)(PMe₃)(η²-1,2-6-cyano-cyclohexadiene) (prepared by a previously reported method)²⁸ (0.088 g, 0.145 mmol) was dissolved in DME (1 mL) and allowed to cool to −30° C. Separately a test tube vial was charged with MeOD (1 mL) and cooled to −60° C. in a toluene bath over a course of 15 min and to this solution was added NaBD₄ (0.050 g, 1.21 mmol) and allowed to stir at −60° C. Next HOTf (0.041 g, 0.273 mmol) was then added at −30° C. to the solution of tungsten in DME and upon addition the reaction mixture turns to a homogeneous yellow color. This solution was allowed to cool to −60° C. over a course of 10 min after being transferred to the −60° C. toluene bath. This yellow reaction mixture was then added dropwise to the solution of NaBD₄/MeOD. This solution was allowed to stir for 16 h at −60° C. before it was removed from the toluene bath. The reaction mixture was diluted with ˜50 mL of Et₂O and loaded onto a 15 mL medium fritted porosity disc that was filled with ˜2 cm of silica and set in Et₂O. The column was then eluted with 100 mL of Et₂O and the solvent was removed in vacuo to reveal a white solid This solid was then dissolved in 2 mL of DCM and added to 15 mL of standing pentane in a 4-dram vial and allowed to sit at −30° C. for 16 hr during which time a white solid precipitated out of solution. The next day the white solid was allowed to triturate in a minimal amount of MeCN given that the desired product was marginally soluble in MeCN. The organic layer was then decanted and the reaction mixture was dried with N₂ (g) and allowed to desiccate for 3 h under active vacuum before a mass was taken (0.038 g, 43% yield).

### Synthesis of 62

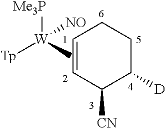

¹H NMR Characterization of 62-4-endo-d₁ matches that of 48 but with a 99% loss of ¹H signal intensity at 2.07 ppm (isotopic purity estimated by ¹H NMR, in this case proton impurity is beyond ¹H NMR detection limit).

To a 4-dram vial added 1 (0.257 g, 0.442 mmol) along with MeCN (2 mL) and this was allowed to sit at −30° C. Separately a solution of NaCN (0.197 g, 4.02 mmol) in MeOD (2 mL) was prepared in a separate 4-dram vial and allowed to stir for 15 min at room temperature before it was transferred to a toluene bath that had been chilled to −40° C. In another 4-dram vial diphenylammonium triflate (DPhAT, 0.168 g, 0.525 mmol) was dissolved in MeOD (2.38 g, 71.9 mmol) to generate an in situ source of acidic deuterium and allowed to sit at −30° C. for 10 min. This acidic deuterium source was then added to the solution of 1 in MeCN at −30° C. and upon addition the reaction mixture turns from a heterogeneous yellow solution to a homogeneous red/orange solution. This reaction mixture was then allowed to sit in the −40° C. toluene bath for 5 min before it was added dropwise to the −40° C. solution of stirring NaCN/MeOD. Upon addition the reaction mixture turns from a homogeneous red solution to a homogeneous yellow solution and this reaction mixture was allowed to stir for 16 h. After 16 h an off-white solid had precipitated out of solution. The solution was filtered through a fine 15 mL fritted porosity disc to yield an off-white solid. This solid was then washed with DI H₂O (3×5 mL) and then pentane (3×5 mL) and allowed to desiccate under active vacuum for 4 h and its identity confirmed by ¹H NMR and a mass was taken (0.112 g, 0.18 mmol). The resulting off white solid was subsequently dissolved in 2 mL of MeOH and cooled to −30° C. over the course of 10 min. Separately a test tube of MeOH was cooled in a −50° C. toluene bath before NaBH₄ (0.117 g, 3.09 mmol) was added to the chilled solution. To the solution of 4-6,endo-d1 was added a −30° C. solution of HOTf (0.061 g, 0.41 mmol) and this reaction mixture was transferred and cooled for 15 min in the −50° C. toluene bath before the reaction mixture was added dropwise to the −50° C. NaBH₄/MeOH solution. This solution was allowed to stir for 16 h at −50° C. before it was removed from the toluene bath. The reaction mixture was diluted with ˜50 mL of Et₂O and loaded onto a 15 mL medium fritted porosity disc that was filled with ˜2 cm of silica and set in Et₂O. A lime green band was collected upon elution with 100 mL of Et₂O and the solvent was removed in vacuo to reveal a lime green solid. This solid was then dissolved in 2 mL of DCM and added to 15 mL of standing pentane in a 4-dram vial and allowed to sit at −30° C. for 16 hr during which time a white solid precipitated out of solution. The next day the white solid was allowed to triturate in a minimal amount of MeCN given that the desired product was marginally soluble in MeCN. The organic layer was then decanted and the reaction mixture was dried with N₂(g) and allowed to desiccate for 3 h under active vacuum before a mass was taken (0.038 g, 14% overall yield).

### Synthesis of 63

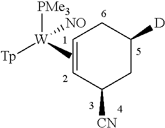

¹H NMR Characterization of 63-5-exo-di matches that of 48 but with an 91% loss of ¹H signal intensity at 1.70 ppm (isotopic purity estimated by ¹H NMR). Although overlap is present with the H5-endo proton, the precursor compound shows suppression of the expected exo-proton.

To a 4-dram vial added 1 (0.250 g, 0.430 mmol) along with MeCN (2 mL) and this was allowed to sit at −30° C. Separately a solution of NaCN (0.200 g, 4.08 mmol) in MeOH (2 mL) was prepared in a separate 4-dram vial and allowed to stir for 15 min at room temperature before it was transferred to a toluene bath that had been chilled to −40° C. In another 4-dram vial diphenylammonium triflate (DPhAT, 0.150 g, 0.469 mmol) was dissolved in MeOH (1 mL) and allowed to sit at −30° C. for 10 min. This acidic solution was added to the solution of 1 in MeCN at −30° C. and upon addition the reaction mixture turns from a heterogeneous yellow solution to a homogeneous red/orange solution. This reaction mixture was then allowed to sit in the −40° C. toluene bath for 5 min before it was added dropwise to the −40° C. solution of stirring NaCN/MeOD. Upon addition the reaction mixture turns from a homogeneous red solution to a homogeneous yellow solution and this reaction mixture was allowed to stir for 16 h. After 16 h an off-white solid had precipitated out of solution. The solution was filtered through a fine 15 mL fritted porosity disc to yield an off-white solid. This solid was then washed with DI H₂O (3×5 mL) and then pentane (3×5 mL) and allowed to desicatte under active vacuum for 4 h and its identity confirmed by ¹H NMR and a mass was taken (0.177 g, 0.291 mmol). This solid was then added to a 4-dram vial with MeOD (3.17 g, 95.9 mmol) to generate a heterogeneous white solution and allowed to cool to −30° C. Separately a 4-dram vial was charged with HOTf (0.088 g, 0.587 mmol) followed by MeOD (0.792 g, 24.0 mmol) and allowed to cool to −30° C. Separately a test tube vial was charged with MeOD (1 mL) and cooled to −50° C. in a toluene bath over a course of 15 min and to this solution was added NaBH₄ (0.130 g, 3.43 mmol) and allowed to stir at −50° C. The 4-dram solution of HOTf/MeOD was then added at −30° C. to the solution of tungsten in MeOD and upon addition the reaction mixture turns to a homogeneous color. This solution was allowed to cool to −50° C. over a course of 10 min and then added dropwise to the solution of NaBH₄/MeOD. This solution was allowed to stir for 16 h at −50° C. before it was removed from the toluene bath. The reaction mixture was diluted with ˜50 mL of Et₂O and loaded onto a 15 mL medium fritted porosity disc that was filled with ˜2 cm of silica and set in Et₂O. A lime green band was collected upon elution with 100 mL of Et₂O and the solvent was removed in vacuo to reveal a lime green solid. This solid was then dissolved in 2 mL of DCM and added to 15 mL of standing pentane in a 4-dram vial and allowed to sit at −30° C. for 16 hr during which time a white solid precipitated out of solution. The next day the white solid was allowed to triturate in a minimal amount of MeCN given that the desired product was marginally soluble in MeCN. The organic layer was then decanted and the reaction mixture was dried with N₂ (g) and allowed to desiccate for 3 h under active vacuum before a mass was taken (0.042 g, 16% overall yield).

### Example 13

### Discussion of Examples 1-12

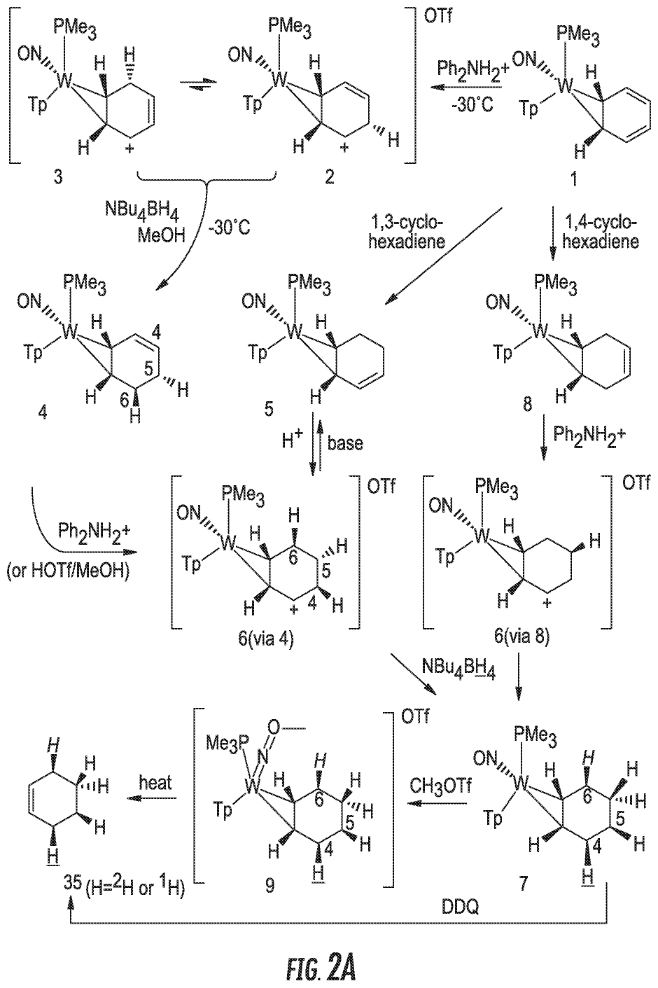

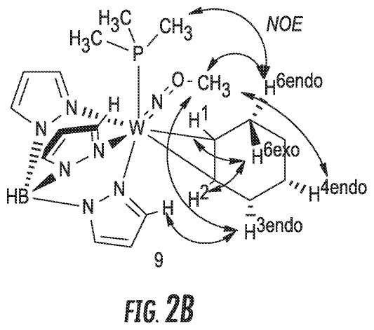

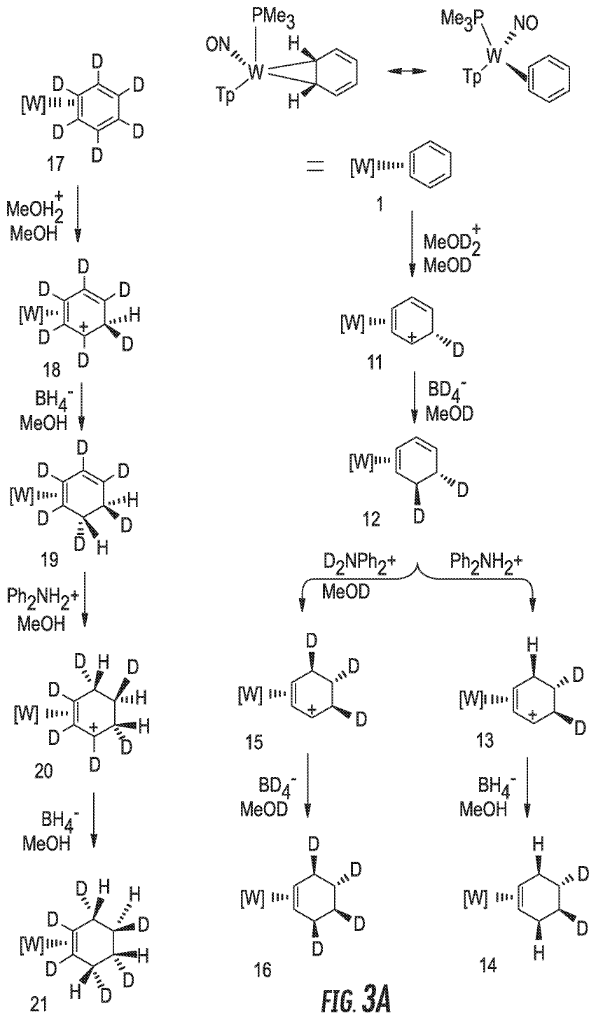

The dearomatization agent {WTp(NO)(PMe₃)} is considerably more activating than its osmium predecessor.⁹ Strong π-backbonding renders arene and diene complexes of this system highly nucleophilic, and resistant to substitution.⁹ Furthermore, this system displays significant electronic asymmetry, and the benzene complex WTp(NO)(PMe₃)(η²-benzene) (1) (see FIG. 1B) can be prepared on a multi-gram scale,¹⁴ and in enantioenriched form.¹⁵ Treatment of an acetone-d₆ solution of 1 with diphenylammonium triflate (DPhAT, pKa ˜0) at −30° C. affords its clean conversion to the η²-benzenium complex [WTp(PMe₃)(NO)(η²-C₆H₇)](OTf) (2). See FIG. 2A. Using chilled diethyl ether as a precipitating solvent, 2 can be isolated from dichloromethane in 86% yield (1.9 g). As an acetonitrile solution, the η²-benzenium complex 2 is moderately stable at room temperature but soon decomposes (t_{1/2}˜6 min). At 0° C., however, 2 exists in equilibrium with its diastereomer 3 in a 10:1 ratio (see FIG. 2A) and persists for three hours without significant decomposition. The major isomer (2) is formed with the metal binding two internal carbons of the five-carbon r-system, and with the newly formed sp³ carbon distal to the PMe₃ ligand. The minor isomer (3) is bound at a terminus of the π-system with the sp³ carbon proximal to the phosphine. Proton NMR data and DFT calculations of these η²-benzenium complexes (2, 3) suggest that they are similar in structure to complexes of the form [WTp(NO)(PMe₃)(η²-allyl)]⁺,¹⁶ where the allyl ligand is tightly bound to the metal through only two carbons. A third carbon, weakly associated to the metal, resembles a carbocation, and is indicated as such in the figures and schemes herein. See FIG. 2A. Combining cold solutions of 2 and tetrabutylammonium borohydride generates WTp(PMe₃)(NO)(η²-1,3-cyclohexadiene) exclusively (4). Despite the coexistence of the allyl conformer 3 in solution, the WTp(PMe₃)(NO)(η²-1,4-cyclohexadiene) complex (8) is undetected in the reaction mixture.¹⁶ The η²-diene complex 4 was then treated with either DPhAT or HOTf/MeOH acids to generate the η²-allyl complex (6).¹⁶ When 6 was subjected to base, it deprotonated to form 5, a stereoisomer of 4,¹⁶ in which the uncoordinated double bond is now distal to the PMe₃.¹⁶ Combining the allyl complex 6 with a hydride source produced the desired η²-cyclohexene complex 7 (67%). Crystals suitable for X-ray structure determinations were grown for complexes of cyclohexadiene 4, allyl complex 6, and cyclohexene 7, and key NOE interactions were determined. Overlapping signals in the ¹H NMR spectrum of cyclohexene complex 7 precluded unambiguous stereochemical assignments of some of the ring proton signals.

By methylating the nitrosyl ligand of 7 (CH₃OTf) to generate [WTp(NOMe)(PMe₃)(η²-C₆H₁₀)]OTf, (9),¹⁷ the chemical shifts of the cyclohexene ring separated to the point that each proton could be assigned with high confidence. An X-ray structure determination of 9 provided conclusive evidence for methylation of the nitrosyl oxygen, analogous to earlier literature reports.¹⁸ Strong NOE interactions between the ring endo protons and the methylated nitrosyl ligand (see FIG. 2B) further facilitated these assignments and quantitative NOE experiments were carried out that support the stereochemical assignments of all diastereotopic protons on the cyclohexene ring (SI, section H).

Deuterium studies: With all hydrogen resonances for the methylated η²-cyclohexene complex 9 fully assigned, the regio- and stereochemical fidelity of the reaction sequence was studied. See FIG. 3A. When the η²-benzenium complex 11 is prepared from 1 using [MeOD₂⁺]OTf, a loss of signal intensity is observed, corresponding to the methylene endo proton. This indicates that protonation of the η²-benzene occurs syn to the metal. See FIG. 3A. A complementary experiment was next performed starting with the fully deuterated benzene complex, 17, in which MeOH⁺ was used as the acid source. In this case, protonation led to a single broad proton resonance for the deuterated η²-benzenium complex 18. This proton signal is ˜0.03 ppm upfield from its proteo counterpart, consistent with a primary H/D isotopic shift.¹⁹ The endo-selective protonation of the benzene ligand in 1 is in stark contrast to the addition of carbon and heteroatom electrophiles, which have been observed to add anti to η²-arene and η²-diene ligands of tungsten complexes.⁹ When η²-benzenium complexes 11 and 18 were treated with NaBD₄ or NaBH₄, respectively, the complementary cyclohexadiene complexes 12 and 19 were formed. See FIG. 3A. A comparison of NOESY data for all three isotopologues of the cyclohexadiene complex (4, 12, 19) confirmed that the proton delivered from the borohydride reagent is anti to the metal. The cyclohexadiene complexes 12 and 19 were then taken forward to their π-allyl analogs 13, 15, and 20. See FIG. 3A. In contrast to protonation of the η²-benzene ligand of 1, the acidic hydrogen was delivered predominantly anti to the metal. See FIG. 3A.

The resulting η²-allyl complexes (13, 15, 20) underwent a conformational change (“allyl shift”) such that the second proton added becomes H₆ₑₓₒ (conversion of 4 to 6, see FIG. 2A), while the first proton added is now H_{5endo}. For allyl complexes 13 and 20, full stereoselective protonation was achieved. However, with the preparation of 15 or 26 we experienced difficulties in achieving full deuterium incorporation, owing to an unusually large DKIE (k_{H}/k_{D}˜37 at −30° C. for the deuteration of 12 or 4). This DKIE was determined for 4 as the average value from three separate experiments in which 26 was generated from acidic solutions with differing H/D ratios. This DKIE could be decreased by raising the temperature to 22° C., however such action compromised the stereofidelity of the resulting deuterated product (15), with endo deuteration of the η²-diene 12 now competing with exo deuteration. Consequently, stereoselective deuterium incorporation at the H₆ₑₓₒ position of cyclohexene (i.e., 16, 33-35, 41, 44, 49, 51; see FIGS. 3A-3C) could not be achieved above ˜75-80%. A similar outcome was observed for conversion of the d₆-isotopologue, diene 19 to allyl 30. Finally, as before, treatment of 13, 15, or 20 with a hydride or deuteride source confirmed that the corresponding η²-cyclohexene products (14, 16, 21) are formed by nucleophilic addition anti to the metal. See FIG. 3A. Similar to the 1,3-diene complex 4, its isomer 5 undergoes exo protonation to form the allyl complex 24. Remarkably, treatment of the 1,4-cyclohexadiene complex (8) with D⁺ (D₂NPh₂⁺ in MeOD) also undergoes direct exo protonation (see FIG. 3B), this time providing allyl 25. The direct exogenous protonation of the unconjugated C═C bond in 8 appears to result in a carbocation that DFT calculations reveal can be stabilized by the participation of the nitrosyl ligand. A subsequent [1,2]-hydride shift results in the formation of the allyl complex 25. Unambiguous assignment of the deuterated hydrogen atom in 25 comes from its conversion to 9-di (via 39). See FIG. 3B. In order to demonstrate regio- and stereocontrol of deuterium incorporation, additional deuterated isotopomers of the allyl complex were prepared from the monodeuterated dienes 22 and 23, and from the benzene-d₆-derived allyls 20 and 31. See FIGS. 3B-3D. The allyl complexes 24-31 were then combined with deuteride or hydride to form 18 additional cyclohexene complexes 32-46, 49-51. In principle, 10 different isotopologues of the cyclohexene complex can be prepared stereoselectively using the procedures outlined above (do-d₄; d₆-d₁₀), eight of which (7, 16, 32-38) are reported herein.

Levels of isotopic purity for the cyclohexene ligand isotopologues were determined by recording high resolution mass spectrometry (HRMS) data for the corresponding complexes as their methylated adducts (analogous to the complex in FIG. 2B) in order to create a suitable cation for ESI mass analysis. Using the isotope envelope of 9-d₀ as a reference, the isotopic purity of 7, 16, and 32-38 (as converted to 9-dₙ) was estimated to be >90%, with the exception of 16 (79%), for which the high DKIE of the second protonation prevented complete deuteration at the H₆ₑₓₒ position. Finally, as a demonstration of how the {WTp(NO)(PMe₃)} system precisely governs both the stereochemistry and regiochemistry of protonation and hydride addition, a series of five monodeuterated (32, 39-42), seven dideuterated (14, 33, 35, 43-46), and four trideuterated (34, 49-51) isotopomers of the cyclohexene complex were prepared from using the presently disclosed methods. See FIGS. 3B and 3C.

Oxidation of the tungsten complex 7 with DDQ releases the free cyclohexene, 35. See FIG. 2A. Such action on 32, 42, 45, and 46 confirmed the expected regiochemistry of these d₁ and d₂ isotopomers of cyclohexene via ¹³C NMR by comparison to the ¹³C NMR of d₀-cyclohexene. Introduction of a single deuterium in 3-deuterocyclohexene or 4-deuterocyclohexene allows one to distinguish all six of the carbons in the ¹³C NMR spectrum, owing to isotopic shifting of the now asymmetric cyclohexene carbons. Alternatively, solvent-free heating of various isotopologues of the methylated complex 9 effected the release of the cyclohexene ligand for analysis by molecular rotational resonance (MRR).²⁰ These experiments determined that 1) over-deuteration is exceedingly low (<2%); 2) the stereoselectivity is excellent when assessed by observation of undesired cis/trans isomers, which in the worst case is 22:1 and in other cases it is 40:1 or higher; and 3) the dominant stereoisotopomers in all cases are those predicted by ¹H NMR data. As a final check of stereochemical assignments, the locations of the deuterium atoms were confirmed for complex 45 by neutron diffraction.

Mechanistic considerations: The reaction of 1 with D⁺ to form 11 results in deuterium incorporation exclusively endo to the metal, but this does not definitively show which carbon is initially protonated. Given that the endo proton of the benzene ligand in 1 completely preempts protonation from an exogenous acid (exo), and without being bound to any one theory, it is believed that the protonation must be concerted—that C—H bond formation is intramolecular and simultaneous with electronic changes at the metal—which could lower the activation barrier for this process relative to protonation by an external acid. Such a mechanism could occur via a hydride intermediate, but this seems sterically untenable. Rather, again without being bound to any one theory, a mechanism (in which the nitrosyl ligand first is protonated to form a hydroxylimido ligand analogous to that reported by Legzdins et al.²¹ This action can be followed by a concerted proton transfer in which a gamma carbon of the benzene is protonated simultaneously with release of electron density back into the tungsten through the NO group. The role of nitrosyl ligands in intramolecular proton transfer has been previously documented.²² In contrast, the stereochemistry and kinetics of η²-diene protonation (e.g., 4 in FIG. 2A) indicates the hydrogen is delivered exogenously, anti to the tungsten. Without being bound to any one theory, it is believed that, while endo protonation can still be accessible for these 1,3-cyclohexadiene complexes, the less-delocalized diene ligand is likely more basic than its η²-benzene predecessor, and its direct exo protonation apparently preempts the purported endo mechanism at −30° C.

Transition metal promoted protonation of benzene was observed in the η⁴-benzene complexes Cr(CO)₃(η⁴-benzene)- and Mn(CO)₃(η⁴-benzene)- by Cooper et al,²³⁻²⁴ and was proposed to occur via hydride intermediates.²³⁻²⁴ More recently, Chirik et al have explored the molybdenum-catalyzed reduction of benzene and cyclohexadiene, with D₂ (g), which resulted in mixtures of isotopologues of cyclohexane.¹² However, reduction of cyclohexene with D₂ produced a single cis isotopomer of 1,2-dideuterocyclohexane using the molybdenum catalyst.

The high stereoselectivity enabled by the tungsten system provides unprecedented control over the preparation of specific isotopologues and isotopomers of cyclohexene, starting from either benzene complex 1 or its deuterated analog 17, and utilizing either proteated or deuterated sources of acids and hydrides. See FIG. 3E. As illustration, consider the d₂ isotopologue of the cyclohexene complex, 7-d₂. Given that the {WTp(NO)(PMe₃)} system is available in enantioenriched form,¹⁵ one has access to 14 different isotopomers (individual enantiomers of 14, 33, 35, 43-46). See Table 1, below. The cyclohexene-d₂ ligand of these complexes, once removed from the metal by oxidative decomplexation, can be available as 11 individual isotopomers: both enantiomers of cis-3,4-, trans-3,4-, cis-3,5-, trans-3,5-, trans-4,5-, and the meso compound cis-3,6-dideuterocycloohexene. Similarly, 11 distinct isotopomers of cyclohexene-d₈ should be available from this methodology starting from benzene-d₆. Regarding cyclohexene complex 7-d₃ and 7-d₇, 8 isotopomers of each would be available, and all 16 of these complexes would yield a unique, chiral cyclohexene (8 cyclohexene-d₃, and 8 cyclohexene-d₇). All totaled, the methodology outlined herein could provide access to 52 unique isotopomers of cyclohexene, as derived from benzene and benzene-d₆. For reference, the total number of isotopomers for cyclohexene is 528.

TABLE 1
Isotopologues and Isotopomers of Cyclohexene Complex 7 and Cyclohexene

| | Complex 7-dₙ | Cyclohexene-dₙ | Cyclohexene-dₙ |
|---|---|---|---|
| Isotopologue | # of Isotopomers | # of Isotopomers | Isotopomers possible |
| d₀ | 2 | 1 | 1 |
| d₁ | 10 | 4 | 5 |
| d₂ | 14 | 11 | 25 |
| d₃ | 8 | 8 | 60 |
| d₄ | 2 | 2 | 110 |
| d₅ | 0 | 0 | 126 |
| d₆ | 2 | 2 | 110 |
| d₇ | 8 | 8 | 60 |
| d₈ | 14 | 11 | 25 |
| d₉ | 10 | 4 | 5 |
| d₁₀ | 2 | 1 | 1 |
| Total: | 72 | 52 | 528 |

The ability of {WTp(NO)(PMe₃)} to be optically resolved on a practical scale and to retain its stereochemical configuration, even when undergoing ligand displacement,¹⁵ also makes it a valuable tool for determining the isotopic pattern of cyclohexene H/D isotopomers produced by other methods.⁸ Consider for example a scenario in which an unknown isotopomer of cyclohexene-d₁ is combined with the resolved form of benzene complex (R)-1 in solution and allowed to undergo ligand exchange. Even though the two faces of the cyclohexene ring will bind to tungsten with equal probability, the ¹H NMR spectrum will be unique for each of the five possible isotopomers. See FIG. 3F. For example, when the benzene complex (R)-1 is combined with (R)-3-deuterocyclohexene, two isotopomers of the resulting cyclohexene complex will be formed as a result of the tungsten complex's inability to chemically differentiate between the two faces of the 3-deuterocyclohexene). A ¹H NMR spectrum will show the 3-exo and the 6-endo positions each at half intensity. In contrast, if S-3-deuterocyclohexene is complexed, then only the 3-endo and 6-exo signals will be diminished. The enantiomeric excess (ee) of the 3-deuterocyclohexene can easily be determined from the relative ratios of the 3-endo and 3-exo signals. In a similar manner (R,R)-trans-3,6-dideuterocyclohexene and its enantiomer can be easily differentiated, both from each other and from the cis isomer. Finally, the (R,R,R,R)-3,4,5,6-tetradeuterocyclohexene stereoisomer shown above can be differentiated from its enantiomer and from the all-cis diastereomers. Of note, it is believed that there is no other practical method to differentiate these cyclohexene stereoisotopomers. A similar approach could be taken for any cyclic alkene (e.g., dehydropiperidines, pyrrolines, cyclopentenes) for which an ¹H NMR spectrum of a fully proteated species can be fully assigned.

Deuterated buildinq blocks for medicinal chemistry: The development of deutetrabenazine, is considered by many as a prelude to a new generation of medicines and therapies that incorporate deuterium into the active pharmaceutical ingredient.⁵ Given that each stereoisotopomer of a biologically active substance will have its own unique pharmacokinetic profile, the ability to stereoselectively deuterate cyclohexene or other medchem building blocks could enable the development of new probes, fragment libraries, and leads for medicinal chemists, as well as providing a new tool for organic and organometallic mechanistic studies. Cyclohexene can be readily converted into perhydroindoles,²⁵ perhydroisoquinolines,²⁶ and azepines.²⁷ However, an inability to chemically differentiate the two alkene carbons or the enantioface of the deuterated cyclohexene limits its potential. But by replacing the benzene ligand in FIG. 2A with a substituted benzene, or by utilizing a non-hydrogenic nucleophile in the conversion of 6 to 7 (see FIG. 2A), a series of 3-substituted cyclohexenes can be prepared with highly defined isotopic patterns.

As a proof of concept, the tungsten-trifluorotoluene complex WTp(NO)(PMe₃)(η²—CF₃Ph),²⁸ which can be elaborated into a 3-(trifluoromethyl)cyclohexene complex (47) analogous to the cyclohexene complex 7 was prepared. See FIG. 4A. Liberation of the cyclohexene from {WTp(NO)(PMe₃)} can be accomplished by a one-electron oxidant such as DDQ, Fe(III), or NOPF₆ in yields ranging from 70-75%.²⁸ Oxidation of 47 (e.g., with DMDO) can generate a cyclohexene that has been previously shown to undergo diastereoselective epoxidation (see FIG. 4C), and would therefore be an attractive building block for medicinal chemistry.²⁹ Repeating the synthesis of 47 with deuteride in the final step yields the cis-6-deutero-3-(trifluoromethyl)cyclohexene complex 52 in 95% yield. Various other isotopologues of 47 and 52 were also prepared (47, 52, 53, 54), and the reaction pattern was found to be similar to that observed for benzene. Exemplary compounds are summarized in FIG. 4D, with synthetic details provided in Example 12. Notably, in the syntheses of 47, 52, 53, 54, protonation at the carbon bearing the CF₃ group ultimately occurs endo to the metal, allowing the CF₃ group to assume an exo stereochemistry. However, if the purported diene intermediate is protonated under kinetic control, exo protonation forces the CF₃ group endo, and the result after a second hydride reduction is the cyclohexene complex 55. Exploiting this reactivity feature, other isotopologues of 55 (i.e., 56 and 57) with inversion of the stereocenter bearing the —CF₃ substituent can be prepared. See FIGS. 4A and 4D.

As further demonstration of the ability of this methodology to selectively prepare isotopomers of functionalized cyclohexenes, the tungsten complex of cis,trans-3-cyano-4,5-dideuterocyclohexene (58) was prepared by the addition of cyanide to the allyl intermediate 13 (57%; dr>98%). See FIGS. 4B and 4C. Other d₁-isotopolouges were also prepared (see FIG. 4B) and the stereochemistry could again be controlled with the sequence of nucleophiles. For example, 58, 59 and 60 could be prepared by generating the appropriate isotopologue of the tungsten-allyl complex and then treating with NaCN. See FIG. 4B. Conversely, treating the benzenium 2 with NaCN leads to a cyano-substituted cyclohexadiene that can be subsequently combined with acid and hydride source to generate other cyclohexene isotopomers (61-63). 3-cyanocyclohexene (proteo form) has been previously used as a precursor to cytotoxic mustards that are of interest in cancer research.³⁰ Allyl-substituted cyclohexenes theoretically exist as 1024 different H/D isotopomers (512 for each enantiomer). Using the tungsten dearomatization methodology, the CF₃- and CN-substituted cyclohexenes are accessible as 64 and 60 unique isotopomers respectively. A full range of both carbon and nitrogen nucleophiles has now been demonstrated to add to tungsten benzenium and allyl tungsten complexes,³¹ which demonstrates the broad scope of compounds that can now be prepared as various deuteroisotopomers.

### Example 14

### Deuterated THP

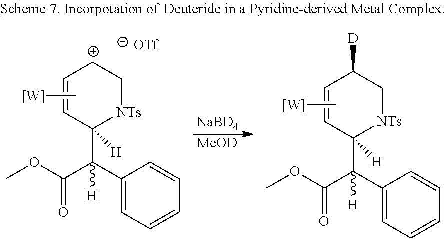

As shown in Scheme 7, above, deuterium was incorporated as a nucleophile into a pyridine-derived metal complex using conditions analogous to those described in Example 11. The deuteration appeared to be regio- and stereoselective. Successful deuteration was supported by simplification of peak splitting in the ¹H NMR spectrum of the deuterated complex as compared to the peak splitting in the ¹H NMR spectrum of the analogous non-deuterated complex, as well as the presence of a 1:1:1: triplet in the ¹³C NMR spectrum (due to C-D coupling). By heteronuclear single quantum coherence (HSQC), the triplet carbon signal can be matched to one of the methylene ¹H signals, the signal for the other methylene proton in the ¹H NMR spectrum is suppressed.

### Example 15

### Deuterated THPallyl

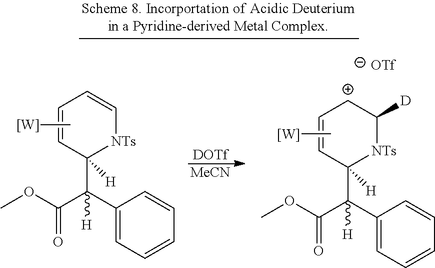

As shown in Scheme 8, above, deuterium was incorporated as acidic deuterium into a pyridine-derived metal complex using conditions analogous to those described in Example 10. The deuteration appeared to be regio- and stereoselective. Successful deuteration was supported by simplification of ¹H NMR signals at 4.65 and 3.84 ppm, and the suppression of ¹H signals at 4.32 and 4.01 (one for each diastereomer of the complex).

### REFERENCES

The following references correspond to superscripted citations present in the instant disclosure. All references listed in the instant disclosure, including but not limited to all patents, patent applications and publications thereof, scientific journal articles, and database entries (including all annotations available therein) are incorporated herein by reference in their entireties to the extent that they supplement, explain, provide a background for, and/or teach methodology, techniques, and/or compositions employed herein.
1. Gant. Using Deuterium in Drug Discovery: Leaving the Label in the Drug. J. Med. Chem. 2014, 57, 3595-3611.
2. Thibblin & Ahlberg. Reaction Branching and Extreme Kinetic Isotope Effects in the Study of Reaction Mechanisms. Chemical Society Reviews 1989, 18, 209-224.
3. Thibblin. Unusually Large Kinetic Deuterium Isotope Effects on Oxidation Reactions. 1. The Mechanism of Hydroxide-Catalysed Permanganate Oxidation of PhCD(CF₃)OH and PhCD(CH₃)OH in Water. Journal of Physical Organic Chemistry 1995, 8, 186-190.
4. Nelson & Trager. The Use of Deuterium Isotope Effects to Probe the Active Site Properties, Mechanism, of Cyctochrome P450-Catalyzed Reactions, and Mechanisms of Metabolically Dependent Toxicity. Drug Metabolism and Disposition 2003, 31, 1481-1497.
5. Dean & Sung. Review of Deutetrabenazine: a Novel Treatment for Chorea Associated with Huntington's Disease. Drug design, development and therapy 2018, 12, 313-319.
6. Loh et al. Photoredox-catalyzed Deuteration and Tritiation of Pharmaceutical Compounds. Science 2017, 358 (6367), 1182-1187.
7. Yu et al. Iron-catalysed Tritiation of Pharmaceuticals. Nature 2016, 529, 195-199.
8. Baldwin et al. Quantitative Analyses of Stereoisomeric 3,4-d₂-Cyclohexenes in the Presence of 3,6-d₂-Cyclohexenes. J. Org Chem. 2009, 74, 3866-3874.
9. Liebov & Harman. Group 6 Dihapto-Coordinate Dearomatization Agents for Organic Synthesis. Chem. Rev. 2017, 117, 13721-13755.
10. Eisen & Marks. Supported organoactinide complexes as heterogeneous catalysts. A Kinetic and Mechanistic Study of Facile Arene Hydrogenation. J. Am. Chem. Soc. 1992, 114, 10358-68.
11. Jones & Seeberger. Synthesis of polymer-supported transition metal catalysts via phosphido linkages: heterogeneous catalysts for the hydrogenation of aromatic compounds under mild conditions. J. Chem. Soc., Chem. Commun. 1985, 373-4.
12. Joannou et al. Pyridine(diimine) Molybdenum-Catalyzed Hydrogenation of Arenes and Hindered Olefins: Insights into Precatalyst Activation and Deactivation Pathways. ACS Catalysis 2018, 8, 5276-5285.
13. Harman & Taube. The selective hydrogenation of benzene to cyclohexene on pentaammineosmium(II). J. Am. Chem. Soc. 1988, 110, 7906-7907.
14. Welch et al. Large-Scale Syntheses of Several Synthons to the Dearomatization Agent {TpW(NO)(PMe₃)} and Convenient Spectroscopic Tools for Product Analysis. Organometallics 2007, 26, 2791-2794.
15. Lankenau et al. Enantioenrichment of a Tungsten Dearomatization Agent Utilizing Chiral Acids. J. Am. Chem. Soc. 2015, 137, 10, 3649-3655.
16. Harrison et al. Hyperdistorted Tungsten Allyl Complexes and Their Stereoselective Deprotonation to Form Dihapto-Coordinated Dienes. Organometallics 2011, 30, 2587-2597.
17. Lis et al. The Uncommon Reactivity of Dihapto-Coordinated Nitrile, Ketone, and Alkene Ligands When Bound to a Powerful π-Base. Organometallics 2006, 25, 5051-5058.
18. Arashiba et al. Electrophilic O-Methylation of a Terminal Nitrosyl Ligand Attained by an Early-Late Heterobimetallic Effect. Organometallics 2006, 25, 560-562.
19. Jamison. Isotope Effects on Chemical Shifts and Coupling Constants 2007, In Encyclopedia of Magnetic Resonance. John Wily & Sons, Ltd. DOI: 10.1002/9780470034590.emrstm0251.
20. Pérez et al. Broadband Fourier transform rotational spectroscopy for structure determination: The water heptamer. Chemical Physics Letters 2013, 571, 1-15.
21. Sharp et al. O-Protonation of a Terminal Nitrosyl Group To Form an η¹-Hydroxylimido Ligand. J. Am. Chem. Soc. 2001, 123, 8143-8144.
22. Llamazares et al. Ligand-Assisted Heterolytic Activation of Hydrogen and Silanes Mediated by Nitrosyl Rhenium Complexes. Organometallics 2001, 20, 5277-5288.
23. Leong & Cooper. Electrophilic activation of benzene in [Cr(η⁴-C₆H₆)(CO)₃]₂. J. Am. Chem. Soc. 1988, 110, 2644-2646.
24. Thompson et al. Reductive activation of the coordinated benzene in manganese complex [Mn(η⁶-C₆H₆)(CO)₃]+: synthesis and characterization of the η⁴-naphthalene complex PPN[Mn(η⁴-C₁₀H₈)(CO)₃]. Organometallics 1991, 10, 1657-1659.
25. Ungureanu et al. Phenylaziridine as a Masked 1,3 Dipole in Reactions with Nonactivated Alkenes. 2000, 39, 4615-4617.
26. Sarkar et al. [4+2] Cycloadditions of N-Alkenyl Iminium Ions: Structurally Complex Heterocycles from a Three-Component Diels-Alder Reaction Sequence. J. Am. Chem. Soc. 2008, 130, 9222-9223.
27. Shim et al. A new and convenient synthesis of N-substituted perhydroazepines from adipaldehyde and primary amines with tetracarbonylhydridoferrate, HFe(CO)₄—, as a selective reducing agent. J. Heterocycl. Chem. 1988, 25, 1383-5.
28. Wilson et al. Sequential Tandem Addition to a Tungsten-Trifluorotoluene Complex: A Versatile Method for the Preparation of Highly Functionalized Trifluoromethylated Cyclohexenes. J. Am. Chem. Soc. 2017, 139, 11401-11412.
29. Murray et al. Diastereoselectivity in the Epoxidation of Substituted Cyclohexenes by Dimethyldioxirane J. Org Chem. 1996, 61, 1830-1841.
30. Leiris et al. Synthesis and biological evaluation of cyclic nitrogen mustards based on carnitine framework. European Journal of Medicinal Chemistry 2010, 45, 4140-4148.
31. Wilson et al. Highly Functionalized Cyclohexenes Derived from Benzene: Sequential Tandem Addition Reactions Promoted by Tungsten. J. Org Chem. 2019, 84, 6094-6116.
32. U.S. Patent Application Publication No. 2009/0076079.
33. Arnold et al. Fast quantitative determination of methylphenidate levels in rat plasma and brain ex vivo by MALDI-MS/MS. Journal of Mass Spectrometry 2015, 50, 963-971.
34. Walter et al. Derivatives of 3-Piperidinol as Central Stimulants. J. Med. Chem. 1968, 11, 792-796.
35. Harrison et al. An efficient synthesis of an η²-pyridine complex and a preliminary investigation of the bound heterocycle's reactivity. J. Am. Chem. Soc. 2008, 50, 16844-16845.

It will be understood that various details of the presently disclosed subject matter may be changed without departing from the scope of the presently disclosed subject matter. Furthermore, the foregoing description is for the purpose of illustration only, and not for the purpose of limitation.

1. A method of preparing an isotopologue or a stereoisotopomer of a cyclic or heterocyclic alkene or diene, the method comprising:
(a) providing a first metal complex, wherein said first metal complex comprises a transition metal selected from tungsten (W), rhenium (Re), osmium (Os), and molybdenum (Mo) and a dihapto-coordinated ligand, wherein said dihapto-coordinated ligand is selected from an arene, a heteroarene or a salt thereof, and an alicyclic compound comprising at least two carbon-carbon double bonds;
(b) reducing the dihapto-coordinated ligand, optionally wherein said reducing comprises contacting said first metal complex sequentially with at least a first reagent and a second reagent, wherein said first reagent is a Bronsted acid or a deuterated or tritiated analogue thereof, and wherein the second reagent is a nucleophilic reagent, thereby forming a second metal complex comprising the transition metal and a dihapto-coordinated cyclic or heterocyclic alkene or diene; and
(c) decomplexing the cyclic or heterocyclic alkene or diene from the second metal complex, wherein said decomplexing optionally comprises contacting the second metal complex with an oxidant, thereby providing the isotopologue or stereoisotopomer of a cyclic or heterocyclic alkene or diene, wherein said isotopologue or stereoisotopomer comprises at least one deuterium or tritium.

2. The method of claim 1, wherein the transition metal is W.

3. The method of claim 2, wherein providing the first metal complex comprises one of:
contacting tungsten trispyrazolylborate nitroso trimethylphosphine dihapto-coordinated benzene (WTp(NO)(PMe₃)(η²-benzene)) with an arene, an alicyclic diene or an alicyclic triene, thereby forming a WTp(NO)(PMe₃)(η²-arene), a WTP(NO)(PMe₃)(η²-diene) or a WTp(NO)(PMe₃)(η²-triene);
contacting a tungsten trispyrazolylborate nitroso trimethylphosphine halide complex with an arene in the presence of an alkali metal, optionally sodium, thereby forming a WTp(NO)(PMe₃)(η²-arene) complex; and
contacting WTp(NO)(PMe₃)(η²-benzene) with a pyridine borane and contacting the resulting complex with a Bronsted acid to form a WTp(NO)(PMe₃)(η₂-pyridinium) salt, optionally followed by contacting the WTp(NO)(PMe₃)(η₂-pyridinium) salt with an anhydride or acid chloride in the presence of a weak base.

4. The method of claim 1, wherein the dihapto-coordinated ligand of the first metal complex is selected from the group consisting of benzene, naphthalene, anthracene, cyclopentadiene, cyclohexadiene, furan, pyrrole, pyridine, a pyridinium salt, thiophene, and deuterated, tritiated, and/or substituted analogues thereof; optionally wherein the arene is selected from the group consisting of benzene, substituted benzene, naphthalene, substituted naphthalene, furan, a pyridinium salt, a substituted pyridinium salt and deuterated or tritiated analogues thereof.

5. The method of claim 1, wherein the first metal complex comprises a dihapto-coordinated arene or a dihapto-coordinated heteroarene or salt thereof, and wherein step (b) comprises:
(b1) contacting the first metal complex sequentially with a first reagent and a second reagent, wherein the first reagent is a Bronsted acid or a deuterated or tritiated analogue thereof, and wherein the second reagent is a nucleophilic reagent, thereby forming an intermediate metal complex comprising a dihapto-coordinated cyclic or heterocyclic diene ligand; and
(b2) contacting the intermediate metal complex comprising the dihapto-coordinated cyclic or heterocyclic diene ligand sequentially with a third reagent and a fourth reagent, wherein the third reagent is a Bronsted acid or a deuterated or tritiated analogue thereof, and wherein the fourth reagent is a nucleophilic reagent; thereby forming the second metal complex, wherein said second metal complex comprises a dihapto-coordinated cyclic or heterocyclic alkene ligand.

6. The method of claim 5, wherein the first reagent and the third reagent are each independently a strong acid or a deuterated or tritiated analogue thereof, wherein said strong acid is selected from the group consisting of diphenylammonium triflate (DPhAT), trifluoromethanesulfonic acid (HOTf); sulfuric acid (H₂SO₄), hexafluorophosphoric acid (HPF₆), tetrafluoroboric acid (HBF₄), hydrochloric acid (HCl), and hydrobromic acid (HBr).

7. The method of claim 6, wherein the contacting with the first reagent in step (b1) and the contacting with the third reagent in step (b2) is performed in an ether, nitrile, or ester solvent at a temperature between about −60° C. and about −20° C., optionally at about −30° C.

8. The method of claim 5, wherein at least one of the second reagent and the fourth reagent is a hydride or a deuteride reagent selected from sodium borohydride (NaBH₄) and sodium borodeuteride (NaBD₄); wherein when the at least one of the second reagent and the fourth reagent is NaBH₄, the contacting with the at least one of the second reagent and the fourth reagent is performed in methanol; and wherein when the at least one of the second reagent and the fourth reagent is NaBD₄, the contacting with the at least one of the second reagent and the fourth reagent is performed in deuterated methanol or a mixture of acetonitrile and 15-crown-5 ether.

9. The method of claim 8, wherein the contacting with the at least one of the second reagent and the fourth reagent is performed at a temperature between about −60° C. and about −20° C., optionally at about −60° C.

10. The method of claim 8, wherein the second reagent and the fourth reagents are each independently selected from a hydride reagent and a deuteride reagent.

11. The method of claim 5, wherein at least one of the second reagent and the fourth reagent is selected from the group consisting of a cyanide salt, an alkoxide salt, an alkynide salt, an alkyl or aryl magnesium halide, a dialkylzinc, an enolate, a phosphine, a primary amine, and a secondary amine.

12. The method of claim 5, wherein at least one of steps (b1) and (b2) comprise a stereoselective addition of at least one of a proton, a deuteron, a triton, or a nucleophile, optionally the stereoselective addition of both a proton, deuteron or triton and a nucleophile, further optionally wherein said nucleophile is a hydride or a deuteride.

13. The method of claim 5, wherein the method provides an isotopologue or a stereoisotopomer having at least about 75% isotopic purity, optionally at least about 90% isotopic purity.

14. The method of claim 5, wherein the dihapto-coordinated ligand of the first metal complex is an N-acylated pyridinium salt, a N-tosylated pyridinium salt, or an N-acylated or N-tosylated substituted pyridinium salt, and the method provides an isotopologue or a stereoisotopomer of a tetrahydropyridine (THP).

15. The method of claim 14, wherein the method further comprises contacting the isotopologue or stereoisotopomer of the THP with a hydrogenation reagent, thereby providing an isotopologue or a stereoisotopologue of a piperidine, optionally wherein the piperidine is methylphenidate.

16. The method of claim 5, wherein the dihapto-coordinated ligand of the first metal complex is an arene selected from benzene, benzene-d₆, a substituted benzene, and an exhaustively deuterated, substituted benzene; and wherein step (b1) comprises:
(b1-i) contacting the first metal complex with a Bronsted acid or a deuterated Bronsted acid, thereby forming a metal complex comprising a dihapto-coordinated benzenium ligand; and
(b1-ii) contacting the metal complex comprising the dihapto-coordinated benzenium ligand with a nucleophilic reagent, thereby forming the intermediate metal complex, wherein said intermediate metal complex comprises a dihapto-coordinated cyclohexadiene ligand;
wherein step (b2) comprises:
(b2-i) contacting the intermediate metal complex with a Bronsted acid or a deuterated Bronsted acid, thereby forming a metal complex comprising a dihapto-coordinated allyl ligand; and
(b2-ii) contacting the metal complex comprising the dihapto-coordinated allyl ligand with a nucleophilic reagent, thereby forming the second metal complex, wherein said second metal complex comprises the dihapto-coordinated cyclohexene ligand; and
wherein step (c) comprises decomplexing the dihapto-coordinated cyclohexene ligand, optionally wherein the decomplexing comprises contacting the second metal complex with an oxidant, thereby providing the isotopologue or stereoisotopomer of a cyclohexene, wherein said isotopologue or stereoisotopomer comprises at least one deuterium.

17. The method of claim 16, wherein one of more of steps (b1-i), (b1-ii), (b2-i), and (b2-ii) are stereoselective.

18. The method of claim 16, wherein the method provides an isotopologue or a stereoisotopomer of a cyclohexene having at least about 75% isotopic purity, optionally at least about 90% isotopic purity.

19. The method of claim 18, wherein the dihapto-coordinated ligand of the first metal complex is benzene or benzene-d₆ and the contacting of step (b1-i) comprises endo-selective protonation or deuteration of the benzene or benzene-d₆ ligand.

20. The method of claim 16, wherein the nucleophilic reagent of step (b1-i) is a hydride or a deuteride reagent and the contacting of step (b1-i) comprises exo-selective addition of a hydride or deuteride to the benzenium ligand.

21. The method of claim 16, wherein the contacting of step (b2-i) comprises exo-selective protonation or deuteration of the cyclohexadiene ligand.

22. The method of claim 16, wherein the nucleophilic reagent of step (b2-ii) is a hydride or a deuteride reagent and the contacting of step (b2-ii) comprises selective addition of a hydride or deuteride to the allyl ligand anti to the metal of the metal complex comprising the dihapto-coordinated allyl ligand.

23. The method of claim 16, wherein the arene is benzene or a substituted benzene and the isotopologue or stereoisotopomer is a d₁-, d₂-, d₃-, or d₄-cyclohexene.

24. The method of claim 16, wherein the arene is benzene-d₆ and the isotopologue or stereoisotopomer is a d₆-, d₇-, or d₈-cyclohexene.

25. The method of claim 16, wherein the arene is a substituted benzene, optionally wherein the substituted benzene comprises a substituent selected from alkyl, perfluoroalkyl, cyano, a sulfone, and a sulfonamide.

26. The method of claim 16, wherein the method further comprises contacting the isotopologue or stereoisotopomer of the cyclohexene with a dioxirane, optionally dimethyldioxirane (DMDO), thereby converting the isotopologue or stereoisoptopomer of the cyclohexene into an epoxide.

27. The method of claim 16, wherein the method provides a stereoisotopomer of a cyclohexene with a stereoselectivity of 22:1 or more.

28. The method of claim 1, wherein the decomplexing comprises contacting the second metal complex with an oxidant, wherein said oxidant is a one electron oxidant, optionally wherein the oxidant is selected from the group consisting of 2,3-dichloro-5,6-dicyano-1,4-benzoquinone (DDQ), an iron (Fe) (III) compound, nitrosonium hexafluorophosphate (NOPF₆), a copper (Cu) (II) salt, silver (Ag) (I) salt, or another oxidant with a potential greater than about 0.5 Volts (V) versus a normal hydrogen electrode (NHE).

29. The method of claim 1, wherein the isotopologue or stereoisotopomer of the cyclic or heterocyclic alkene or diene is a synthetic intermediate of a deuterated active pharmaceutical ingredient.

30. An isotopologue or stereoisotopomer prepared according to the method of claim 1.

31. An isotopologue or stereoisotopomer of a cyclohexene or a substituted cyclohexene, wherein said isotopologue or stereoisotopomer comprises at least one cyclohexene ring carbon substituted by hydrogen and at least one cyclohexene ring carbon substituted by deuterium or tritium, subject to the proviso that said isotopologue or stereoisotopomer is not cyclohex-1-ene-1,2-d₂;
cyclohex-1-ene-1-d; (R)-cyclohex-1-ene-3-d; or (3R,4R,5S,6S)-cyclohex-1-ene-3,4,5,6-d₄.

32. The isotopologue or stereoisotopomer of claim 31, wherein said isotopologue or stereoisotopomer has an isotopic purity of at least 75%.

33. The isotopologue or stereoisotopomer of claim 31, wherein said isotopologue or stereoisotopomer is a stereoisotopomer having an enantiomeric excess of about 80% or more.

34. The isotopologue or stereoisotopomer of claim 31, wherein the isotopologue or stereoisotopomer has a structure of one of Formulas (Ia), (Ib), (IIa), (IIb), (IIIa), and (IIIb): 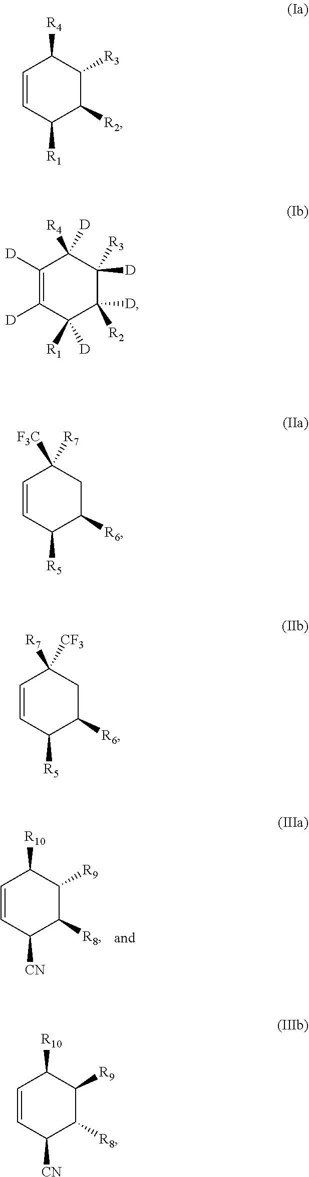
wherein:
each of R₁, R₂, R₃, R₄, R₅, R₆, R₇, R₈, R₉, and R₁₀ is independently selected from H and D, subject to the proviso that for Formula (Ia), at least one of R₁-R₄ is D; that for Formula (Ib), at least one of R₁-R₄ is H; that for Formulas (IIa) and (IIb) at least one of R₅-R₇ is D; and that for Formula (IIIa) and (IIIb), at least one of R₈-R₁₀ is D.

35. The isotopologue or stereoisotopomer of claim 34, wherein the isotopologue or stereoisotopomer has a structure of Formula (Ia): 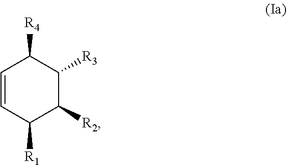
wherein one, two, three, or all four of R₁ R₂, R₃, and R₄ is D.

36. The isotopologue or stereoisotopomer of claim 34, wherein the isotopologue or stereoisotopomer has a structure of Formula (Ib): 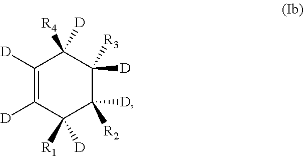
wherein:
R₁ and R₂ are D and R₃ and R₄ are H;
R₁ is D and R₂, R₃, and R₄ are each H; or
R₁-R₄ are each H.

37. The isotopologue or stereoisotopomer of claim 34, wherein the isotopologue or stereoisotopomer has a structure of Formula (IIa): 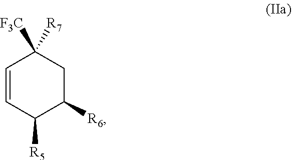
wherein one or both of R₅ and R₆ is D and R₇ is H.

38. The isotopologue or stereoisotopomer of claim 34, wherein the isotopologue or stereoisotopomer has a structure of Formula (IIb): 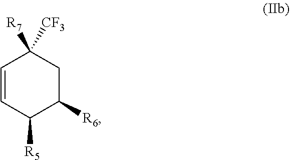
wherein R₅ and R₆ are each D and R₇ is H or D.

39. The isotopologue or stereoisotopomer of claim 34, wherein the isotopologue or stereoisotopomer has a structure of Formula (IIIa): 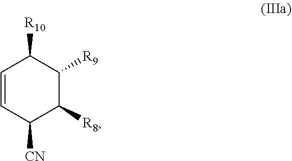
wherein one of R₈-R₁₀ is D and the other two of R₈-R₁₀ are each H; or
wherein R₈ and R₉ are each D and R₁₀ is H.

40. The isotopologue or stereoisotopomer of claim 34, wherein the isotopologue or stereoisotopomer has a structure of Formula (IIIb): 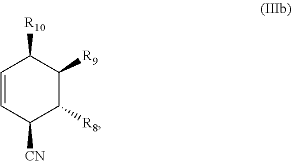
wherein
R₁₀ is H; and
one of R₈ and R₉ is D and one of R₈ and R₉ is H.

41. An isotopologue or stereoisotopomer of tetrahydropyridine or a substituted tetrahydropyridine, wherein the isotopologue or stereoisotopomer comprises one, two, three, four, five, six, or seven deuteriums attached to tetrahydropyridine ring carbon atoms.

42. The isotopologue or stereoisotopomer of claim 41, wherein the isotopologue or stereoisotopomer has a structure of one of Formulas (IVa) and (IVb): 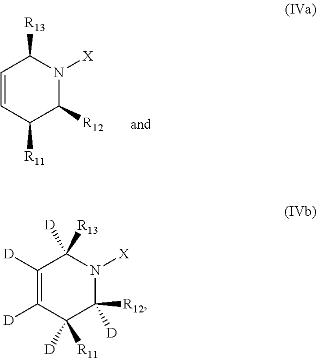
wherein:
X is H, D, acyl, or tosyl; and
each of R₁₁, R₁₂, and R₁₃ is independently selected from H and D, subject to the proviso that for Formula (IVa), at least one of R₁₁, R₁₂, and R₁₃ is D; and for Formula (IVb), at least one of R₁₁, R₁₂, and R₁₃ is H;
or a salt thereof.

43. The isotopologue or stereoisotopomer of claim 41, wherein the isotopologue or stereoisotopomer has a structure of one of (Va) and (Vb): 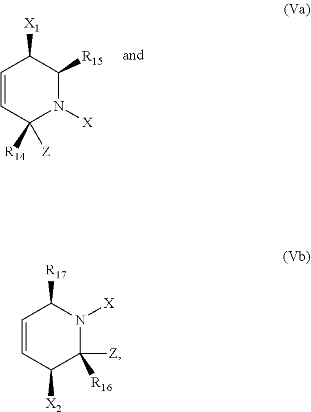
wherein:
X is H, D, acyl, or tosyl;
X₁ and X₂ are each selected from the group consisting of H, D, CN, alkyl, substituted alkyl, alkoxy, aryloxy, —NHR₂₄, —N(R₂₄)₂; and —P(R₂₄)₃;
Z has a structure of the formula: 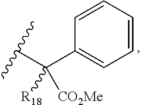
and
each of R₁₄, R₁₅, R₁₆, R₁₇, and R₁₈ is independently selected from H and D; and
each R₂₄ is independently selected from alkyl, aralkyl, and aryl;
subject to the proviso that for Formulas (Va) at least one of R₁₄, R₁₅, and X₁ is D; and that for Formula (Vb) at least one of R₁₆, R₁₇, and X₂ is D;
or a salt thereof.

44. The isotopologue or stereoisotopomer of claim 41, wherein said isotopologue or stereoisotopomer an isotopic purity of at least 75%.

45. An isotopologue or a stereoisotopomer of methylphenidate or a 6-trifluoromethyl substituted derivative thereof, wherein the isotopologue or stereoisotopomer has a structure of Formula (VI): 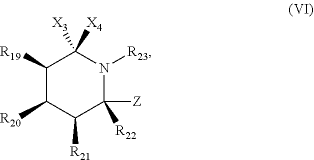
wherein
X₃ and X₄ are each selected from H, D, and —CF₃;
Z has a structure of the formula: 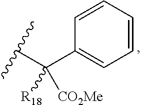
and
each of R₁₈, R₁₉, R₂₀, R₂₁, R₂₂ and R₂₃ is selected from H and D;
or a salt thereof; and subject to the proviso that when one of X₃ and X₄ is —CF₃, the other of X₃ and X₄ is H or D; and that when neither of X₃ and X₄ is —CF₃, X₃ is H and X₄ is H or D; and that at least one of R₂₁, R₂₂, and X₄ is D.

46. The isotopologue or stereoisotopomer of claim 45, wherein said isotopologue or stereoisotopomer an isotopic purity of at least 75%.

47. A method of determining an absolute configuration of a stereoisotopomer of a cyclohexene, wherein the method comprises:
(a) contacting the stereoisotopomer of the cyclohexene with a tungsten metal complex, wherein said tungsten metal complex is a resolved form of WTp(NOMe)(PMe₃)(η²-benzene) and wherein the contacting results in ligand exchange between the benzene and the cyclohexene, thereby providing a tungsten metal complex wherein the stereoisotopomer of the cyclohexene is dihapto-coordinated to tungsten;
(b) collecting a proton nuclear magnetic resonance (NMR) spectrum of the tungsten metal complex comprising the dihapto-coordinated stereoisotopomer of the cyclohexene; and
(c) comparing the proton NMR spectrum collected in step (b) to a proton NMR spectrum of the corresponding tungsten metal complex wherein the dihapto-coordinated ligand is a non-isotopically enriched cyclohexene; thereby determining the absolute configuration of the stereoisotopomer.
